(19) United States
(12) Patent Application Publication
(10) Pub. No.: US 2024/0002535 A1
(43) Pub. Date: Jan. 4, 2024

(54) ARGINASE 1 BINDERS FOR INHIBITING ARGINASE 1 ACTIVITY

(71) Applicant: MERCK SHARP & DOHME LLC, Rahway, NJ (US)

(72) Inventors: Marc Andre Bailly, San Mateo, CA (US); Kalyan Chakravarthy, Woburn, MA (US); Ghassan Najib Fayad, Waban, MA (US); Laurence Fayadat-Dilman, San Carlos, CA (US); Veronica Juan, Redwood City, CA (US); Esther Kofman, San Jose, CA (US); Masahisa Handa, Berkeley, CA (US); Jennifer E. O'Neil, Dedham, MA (US); Rachel Lynn Palte, Melrose, MA (US); Giovanna Scapin, New York, NY (US); Shahriar Shane Taremi, Cambridge, MA (US)

(73) Assignee: MERCK SHARP & DOHME LLC, Rahway, NJ (US)

(21) Appl. No.: 18/253,709

(22) PCT Filed: Nov. 22, 2021
(86) PCT No.: PCT/US2021/060242

§ 371 Date: May 19, 2023

Related U.S. Application Data
(60) Provisional application No. 63/119,272, filed on Nov. 30, 2020.

(51) Int. Cl. C07K 16/40

(52) U.S. Cl. CPC C07K 16/40; A61K 2039/505

(57) ABSTRACT

Arginase 1 binders that inhibit activity of hArg1, which comprise humanized anti-human Arginase 1 (hArg1) antibodies and antigen-binding fragments thereof obtained from mouse monoclonal antibodies comprising mouse VH and VL, are described. These Arginase 1 binders present an alternative mechanism for inhibiting hArg1 activity and highlight the ability to utilize binders as probes in the discovery and development of peptide and small molecule inhibitors for enzymes in general.

### BACKGROUND OF THE INVENTION

### (1) Field of the Invention

The present invention relates to Arginase 1 binders that inhibit activity of human arginase 1 (hArg1), which comprise humanized anti-hArg1 antibodies and antigen-binding fragments thereof obtained from mouse monoclonal antibodies.

### (2) Description of Related Art

Human Arginase 1 is a metalloenzyme that catalyzes the hydrolysis of L-arginine to L-ornithine and urea and is a critical endogenous regulator of the immune system and a key player in T-cell function. This enzyme is constitutively expressed by myeloid derived suppressor cells (MDSCs), which are known immune system regulators. MDSCs have emerged as a key mediator of immunosuppression in human T-cells biology leading to significant decreases in the induction of antitumor activity (Bronte et al., J. Immunother. 24, 431-446 (2001); Kusmartsev. & Gabrilovich, Cancer Immunol. Immunother. 51, 293-298 (2002); Serafini et al., Semin. Cancer Biol. 16, 53-65 (2006); Kumar et al., Trends Immunol. 37, 208-220 (2016)). hArg1 catalyzes the degradation of the conditionally essential amino acid L-arginine into L-ornithine and urea in the final step of the urea cycle (Kumar et al., Ibid; Bronte et al., Trends Immunol. 24, 301-305 (2003); Rodriguez et al., Cancer Res. 64, 5839-5849 (2004)). This enzyme is present both intracellularly and excreted into the extracellular environment in a paracrine manner, with extracellular hArg1 maintaining its ability to deplete L-arginine (Pudlo et al., Med. Res. Rev. 37, 475-513 (2017); Sahin et al., J. Immunol. 193, 1717-1727 (2014); Munder, Br. J. Pharmacol. 158, 638-651 (2009); Wu et al., Amino Acids 37, 153-168 (20089)). As T-cells are dependent on L-arginine for growth and proliferation, its depletion leads to the effective suppression of T-cell immune responses and consequently supports the proliferation of tumor cells both in vitro and in vivo (Kumar et al. op. cit.; Rodriquez et al., op. cit. Activation of lymphocytes, specifically T-cells, via therapeutics targeted at immune checkpoint molecules enhances tumor cell killing and has led to long-lasting responses across various cancers (Wei et al., Cancer Discov. 8, 1069-1086 (2018)). High levels of hArg1 activity have been correlated with various types of cancer (Rodriquez et al., op. cit.; Kusmartsev & Gabrilovich, Cancer Immunol Immunother. 55, 237-245 (2006)).

Human arginase 1 is a trimeric metalloenzyme in which each monomer is approximately 35 kDa in size with an extended, narrow active site approximately 15 A deep that is terminated by two catalytic manganese (Mn) ions 3.3 A apart (Ash, J. Nutr. 134, 2760-2764 (2004)). Several residues within the active site are critical for bridging the two Mn ions and in binding L-arginine (Ash, op. cit.; Costanzo et al., Proc. Natl. Acad. Sci. U. S. A. 102,13058-13063 (2005) and have been the main target of small molecule inhibitors (Ilies et al., J. Med. Chem. 54,5432-5443 (2011); Cox et al., Nat. Struct. Biol. 6,1043-1047 (1999); Van Zandt & Jagdmann Jr., Ring Constrained Analogs As Arginase Inhibitors. 1-21 (2015); Van Zandt et al., J. Med. Chem. 62,8164-8177 (2019); Van Zandt et al., J. Med. Chem. 56,2568-2580 (2013); Mitcheltree et al., ACS Med. Chem. Lett. 11,582-588 (2020);Steggerda et al., Cancer 5,1-18 (2017)). Efforts to discover pharmacological agents to inhibit hArg1 have been focused on amino acid-derived small molecules of usually less than 350 Da (Pudlo, op. cit.) that are able to enter and bind to residues within the hArg1 (Costanzo, op. cit.) active site. One avenue not previously described in literature for hArg1 inhibition is the use of therapeutic antibodies.

Monoclonal antibodies (mAbs) both in monotherapy and in combination regiments has emerged as one of the fastest growing and most effective therapeutic strategies for the treatment of solid tumors and hematological diseases. Between 2015 and 2017, the U.S. Food and Drug Administration approved 27 therapeutic mAbs (Tsumoto et al., Immunotherapy 11,119-127 (2019)) increasing the total of clinically used mAbs and biosimilars in 2017 to 57 and 11, respectively (Grilo & Mantalaris, Trends Biotechnol. 37,9-16 (2019)). As of late 2019, numerous companies were supporting over 550 novel antibody therapeutics in early phase clinical trials, with approximately half of these against oncology targets (Kaplon et al., MAbs 12, 1-24 (2020)).

Numerous published studies focused on the use of antibody fragments such as nanobodies, antigen-binding fragments (Fabs), and single-chain variable domain fragments (scFvs) as potent inhibitors of enzyme activity 25-34 (Dahms et al., Sci. Rep. 6,1-7 (2016); Ganesan et al., Structural and mechanistic insight into how antibodies inhibit serine proteases. Biochem. J. 430,179-189 (2010); Cinader. & Lafferty, Immunology 7,342-362 (1964); Lauwereys et al., EMBO J. 17,3512-3520 (1998); Holliger & Hudson, Nat. Biotechnol. 23, 1126-1136 (2005); Remy et al., Eur. J. Biochem. 231,651-658 (1995); Oyen et al., J. Mol. Biol. 407,138-148 (2011); Ganesan, Structure 17,1614-1624 (2009); De Genst et al., Proc. Natl. Acad. Sci. U. S. A. 103,4586-4591 (2006); Koschubs, T. et al. Biochem. J. 442,483-494 (2012)). The proposed mechanisms of inhibition by antibodies include adaptation to the catalytic site; adaptation to a site other than but near to the catalytic center thereby causing steric hindrance; aggregation of the antigen-antibody complex leading to steric hindrance by the structure of the aggregate; and interference with multimerization that may inhibit enzyme activity (Cinader, Annu. Rev. Microbiol. 11,371-390 (1957)). Nevertheless, as noted by others, despite the myriad antibodies that have been and could be developed, the number of full-length monoclonal antibodies acting as enzyme inhibitors is “disappointingly low.” (Lauwereys, op. cit.). MAbs excel in their ability to bind an antigen with high specificity and potency and function mainly by binding to large, flat surfaces on some receptors and protein:protein interaction surfaces that traditional small molecules cannot bind with suitable potency. Full-length neutralizing antibodies often lack the ability to access the narrow clefts and active site pockets of traditional enzymes due to their larger size, which often eliminates the ability to inhibit enzymatic activity.

Arginase-targeted therapies have been pursued across several disease areas including immunology, oncology, nervous system dysfunction, and cardiovascular dysfunction and diseases. Currently, all published hArg1 inhibitors are small molecules usually less than 350 Da in size.

### BRIEF SUMMARY OF THE INVENTION

The present invention provides Arginase 1 binders that inhibit human arginase 1 (hArg1) comprising full-length humanized anti-hArg1 antibodies and antigen-binding fragments thereof comprising humanized heavy chain variable domain (VH) and light chain variable domain (VL). These Arginase 1 binders inhibit hArg1 through orthosteric and allosteric mechanisms and may be useful for treating cancers and proliferative diseases.

The present invention provides an Arginase 1 binder comprising (a) three complementarity determining regions (CDRs) of an antibody VH comprising the amino acid sequence set forth for VH1 in SEQ ID NO: 2 or VH2 in SEQ ID NO: 3; and (b) the three CDRs of an antibody VL comprising the amino acid sequence set forth for VL1 in SEQ ID NO: 4 or VL2 in SEQ ID NO: 5. CDR sequences can be determined using a Kabat, Chothia, Kabat+Chothia, AbM, ImMunoGeneTics (IMGT), or Contact numbering scheme.

In a further embodiment of the Arginase 1 binder, the Arginase 1 binder specifically binds to two arginase 1 trimers to form a complex comprising three Arginase 1 binders and two arginase trimers, and inhibits arginase 1 activity.

In a further embodiment of the Arginase 1 binder, (a) the VH CDRs comprise a VH1-CDR1 or VH2-CDR1 comprising the amino acid sequence set forth in SEQ ID NO: 6, a

VH1-CDR2 or VH2-CDR2 comprising the amino acid sequence set forth in SEQ ID NO: 7, and a VH1-CDR3 or VH2-CDR3 comprising the amino acid sequence set forth in SEQ ID NO: 8; and (b) the VL CDRs comprise a VL1-CDR1 or VL2-CDR1 comprising the amino acid sequence set forth in SEQ ID NO: 22, a VL1-CDR2 or VL2-CDR2 comprising the amino acid sequence set forth in SEQ ID NO: 23, and a VL1-CDR3 or VL2-CDR3 comprising the amino acid sequence set forth in SEQ ID NO: 24, wherein the CDR sequences are defined by Kabat.

In a further embodiment, the Arginase 1 binder comprises an antibody or antigen-binding fragment VH1 comprising the amino acid sequence set forth in SEQ ID NO: 2 and a VL1 comprising the amino acid sequence set forth in SEQ ID NO: 4. In a further embodiment, the Arginase 1 binder comprises an antibody or antigen-binding fragment VH2 comprising the amino acid sequence set forth in SEQ ID NO: 3 and a VL2 comprising the amino acid sequence set forth in SEQ ID NO: 5.

In a further embodiment, the Arginase 1 binder comprises an antibody comprising a heavy chain constant domain of the IgG1, IgG2, IgG3, or IgG4 isotype. In a further embodiment of the Arginase 1 binder, the antibody comprises a heavy chain constant domain of the IgG1, IgG2, IgG3, or IgG4 isotype wherein the Fc domain comprises one or more mutations that render the Fc domain effector silent.

In a further embodiment, the light chain may comprise a human kappa light chain constant domain or a lambda light chain constant domain. In further embodiments disclosed herein, the light chain may comprise a human kappa light chain constant domain comprising SEQ ID NO: 58 or a lambda light chain constant domain comprising SEQ ID NO: 64. The present invention provides an Arginase 1 binder that is an antibody or an antigen binding fragment thereof comprising two identical Fabs, a first Fab comprising a first heavy chain variable domain (VH) and a first light chain variable domain (VL) and a second Fab comprising a second VH and a second VL, wherein the Arginase 1 binder binds to two arginase 1 trimers, a first arginase 1 trimer and a second arginase 1 trimer, each comprising three arginase 1 monomers, wherein the first Fab binds to an epitope of two adjacent monomers of the first trimer and the second Fab binds to an epitope of two adjacent monomers of the second trimer, wherein (i) the VH of the first Fab binds to a portion of the epitope located solely on one monomer of two adjacent monomers of the first arginase 1 trimer and the VL of the first Fab binds to a portion of the epitope that spans the two adjacent monomers of the first arginase 1 trimer, and (ii) the VH of the second Fab binds to a portion of the epitope located solely on one monomer of two adjacent monomers of the second arginase 1 trimer and the VL of the second Fab binds to a portion of the epitope that spans the two adjacent monomers of the second arginase 1 trimer. The 2:3 ratio of two trimers to three antibodies may be identified using both isothermal titration calorimetry (ITC) and size exclusion chromatography with multi-angle light scattering (SECMALS) and/or cryo-electron microscopy.

In a further embodiment, VH comprises a VH1-CDR1 or VH2-CDR1 comprising the amino acid sequence set forth in SEQ ID NO: 6, a VH1-CDR2 or VH2-CDR2 comprising the amino acid sequence set forth in SEQ ID NO: 7, and a VH1-CDR3 or VH2-CDR3 comprising the amino acid sequence set forth in SEQ ID NO: 8; and VL comprises a VL1-CDR1 or VL2-CDR1 comprising the amino acid sequence set forth in SEQ ID NO: 22, a VL1-CDR2 or VL2-CDR2 comprising the amino acid sequence set forth in SEQ ID NO: 23, and a VL1-CDR3 or VL2-CDR3 comprising the amino acid sequence set forth in SEQ ID NO: 24, wherein the CDR sequences are defined by Kabat.

In further embodiments, and VH is VH1 or VH2 and VL is VL1 or VL2 with the proviso that when VH is VH1 then VL is VL1 and when VH is VH2 then VL is VL2, wherein

VH1, VL1, VH2, and VL2 comprise the amino acid sequence set forth in SEQ ID NO: 2, 3, 4, and 5, respectively.

In a further embodiment of the Arginase 1 binder, (a) the VH of the first Fab and the second Fab each binds to amino acids Glu26, Lys33, Ala34, Glu38,Pro280, Pro286, Glu26, Ser281, Gly283, Lys284, Thr285, Glu287, Thr290, and Val293 of one monomer of the two adjacent monomers of the first arginase 1 trimer and second arginase 1 trimer, respectively; and (b) the VL of the first Fab and the second Fab each binds to (i) amino acids Lys33, Ala34, Gly35, Glu38, Lys39, Lys41, Glu42, Glu287, Thr290, and Asn294 of a monomer (the first monomer) of the two adjacent monomers of the first arginase 1 trimer and second arginase 1 trimer, respectively, and (ii) amino acids Arg21, Lys68, Thr136, Asp181, Val182, Asp183, Pro184, Gly245, Pro247, and Lys284 of another monomer (the second monomer) of the two adjacent monomers of the first arginase 1 trimer and second arginase 1 trimer, respectively.

In a further embodiment of the Arginase 1 binder, the Arginase 1 binder binds two arginase 1 trimers to form a complex comprising a three to two ratio of Arginase 1 binder to arginase 1 trimer.

The present invention further provides an Arginase 1 binder comprising an antibody tetramer comprising two HC+LC pairs, wherein each HC and LC comprises a VH and a VL, respectively, and each VH and VL of a HC+LC pair forms a Fab to provide a first Fab comprising the VH and VL of one of the two HC+LC pairs and a second Fab comprising the VH and VL of the other of the two HC+LC pairs, and wherein the VH of the first Fab and the second Fab each binds to amino acids Glu26, Lys33, Ala34, Glu38, Pro280, Ser281, Gly283, Lys284, Thr285, Pro286, Glu287, Thr290, and Val293 of the first monomer of two adjacent monomers of a first arginase 1 trimer and a second arginase 1 trimer, respectively, and the VL of the first Fab and the second Fab each binds to (ii) amino acids Lys33, Ala34, Gly35, Glu38, Lys39, Lys41, Glu42, Glu287, Thr290, and Asn294 of one monomer of the two adjacent monomers of the first arginase 1 trimer and second arginase 1 trimer, respectively and (ii) amino acids Arg21, Lys68, Thr136, Asp181, Val182, Asp183, Pro184, Gly245, Pro247, and Lys284 of the second monomer of the two adjacent monomers of the first arginase 1 trimer and the second arginase 1 trimer, respectively;

The present invention further provides an Arginase 1 binder that binds two adjacent monomers of an Arginase 1 trimer, wherein the Arginase 1 binder comprises a VH and a VL, wherein (i) the VH binds to the first monomer by interacting with amino acids Glu26, Lys33, Pro280, Pro286, Glu26, Ser281, Gly283, Lys284, Thr285, Pro286, Pro286, Glu287, Lys33, Lys33, Ala34, Thr290, Va1293, and Glu38 of the first monomer and (ii) the VL binds to the first monomer by interacting with amino acids Glu42, Lys39, Glu38, Lys33, Ala34, Gly35, Glu38, Glu38, Lys41 Lys39, Glu287, Thr290, and Asn294 of the first monomer and amino acids Arg21, Arg21, Lys68, Arg21, Gly245, Pro247, Asp181, Val182, Asp183, Pro184, Thr136, and Lys284 of the second monomer.

The present invention further provides an Arginase 1 binder comprising a VH comprising an amino acid sequence with at least 90% identity to the amino acid sequence set forth in SEQ ID NO: 3 and further comprising amino acids Thr30, Tyr33, Val50, Ser52, Tyr54, Asn55, Gly57, Thr59, Asp99, Leu100, Tyr101, and Tyr102, each amino acid position corresponding to the position shown in SEQ ID NO: 3; and a VL comprising an amino acid sequence with at least 90% identity to the amino acid sequence set forth in SEQ ID NO: 5 and further comprising amino acids Glul, Val3, Arg24, Ser26, Gln27, Arg28, Asn31, Asp32, Lys49, Tyr50, Gln53, Asn92, Asp70, and Trp94, each amino acid position corresponding to the position shown in SEQ ID NO: 5.

The present invention further provides a composition comprising an Arginase 1 binder disclosed herein and a pharmaceutically acceptable carrier.

The present invention further provides a method for treating cancer or proliferative disease in an individual in need of the treatment comprising administering to the individual a therapeutically effective amount an Arginase 1 binder disclosed herein or a composition disclosed herein to treat the cancer or a proliferative disease.

The present invention further provides an arginase 1 binder or composition disclosed herein for treatment of cancers or proliferative diseases.

The present invention further provides for the use of an Arginase 1 binder disclosed herein for the manufacture of a medicament for treating cancer or proliferative disease.

The present invention further provides a combination therapy for treating a cancer or proliferative disease comprising an arginase 1 binder or composition disclosed herein and a therapeutic agent. In a further embodiment, the therapeutic agent is a chemotherapy agent or a therapeutic antibody. In a further still embodiment, the antibody is an anti-PD1 or anti-PD-L1 antibody.

The present invention further provides a nucleic acid molecule encoding the VH1 or VH2 of an Arginase 1 binder disclosed herein and/or VL1 or VL2 of an Arginase 1 binder disclosed herein. Further provided is an expression vector comprising one or more of the nucleic acid molecules disclosed herein. Further provided is host cell comprising an expression vector comprising one or more of the nucleic acid molecules disclosed herein.

The present invention further provides a method for producing an Arginase 1 binder comprising (a) providing a host cell comprising an expression vector comprising one or more of the nucleic acid molecules disclosed herein; (b) cultivating the host cell in a medium under conditions suitable for expressing the Arginase 1 binder; and (c) isolating the Arginase 1 binder from the medium.

In a further embodiment, the Arginase 1 binder further comprises a heavy chain constant domain of the IgG1, IgG2, IgG3, or IgG4 isotype.

In a further embodiment, the heavy chain constant domain of the IgG1, IgG2, IgG3, or IgG4 isotype comprises an Fc domain comprising one or more mutations that render the Fc domain effector-silent.

In any one of the embodiments disclosed herein, the light chain may comprise a human kappa light chain constant domain or a lambda light chain constant domain. In any one of the embodiments disclosed herein, the light chain may comprise a human kappa light chain constant domain comprising SEQ ID NO: 58 or a lambda light chain constant domain comprising SEQ ID NO: 64.

### BRIEF DESCRIPTION OF THE DRAWINGS

FIG. 1A. Shows the CDRs of the VH1 domain comprising the amino acid sequence set forth in SEQ ID NO: 2 and the VH2 domain comprising the amino acid sequence set forth in SEQ ID NO: 3 as defined according to Kabat and Chothia. The amino acids in the affinity matured VH2that differ from the amino acids in the parent VH1 are shown in bold.

FIG. 1B. Shows the CDRs of the VH1domain comprising the amino acid sequence set forth in SEQ ID NO: 2 and the VH2 domain comprising the amino acid sequence set forth in SEQ ID NO: 3 as defined according to Kabat+Chothia and AbM. The amino acids in the affinity matured VH2that differ from the amino acids in the parent VH1 are shown in bold.

FIG. 1C. Shows the CDRs of the VH1 domain comprising the amino acid sequence set forth in SEQ ID NO: 2 and the VH2 domain comprising the amino acid sequence set forth in SEQ ID NO: 3 as defined according to IMGT and Contact. The amino acids in the affinity matured VH2 that differ from the amino acids in the parent VH1 are shown in bold.

FIG. 1D. Shows the CDRs of the VL1 domain comprising the amino acid sequence set forth in SEQ ID NO: 4 and the VL2 domain comprising the amino acid sequence set forth in SEQ ID NO: 5 as defined according to Kabat, Chothia, Kabat+Chothia, and AbM; IMGT; and Contact. The amino acids in the humanized VL2 that differ from the amino acids in the mouse VL1 are shown in bold.

FIG. 2. Shows isothermal titration calorimetry (ITC) assay results for mAb4. The calculated stoichiometry is n=0.637, which is consistent with a 2:3 ratio of [hArg1 ;[mAb4].

FIG. 3A. The overall complex formation and epitope mapping for the mAb4 2:3 complex is shown, which provides an overview of how the large 2:3 hArg1 to mAb4 complex assembles. The three monomers of the hArg1 trimer are shown as monomers A, B, and C, and the mAb4 heavy chain and light chain are colored dark grey and light grey, respectively. The left-hand image shows the protein surfaces and the righthand image shows half of the complex in cartoon form as viewed from the top down for ease of interpretation. The Fc is not readily visible in these views but the oval representing an FC is shown for orientation. Here it is apparent that each HC interacts with only one hArg1 monomer while each LC interacts with two hArg1 monomers and that these interactions are symmetric around the trimer.

FIG. 3B. The LC CDR-1 loop is in close proximity to the hArg1 active site with Arg28 (sticks) of the loop extending into the binding pocket and Gln27 (sticks) of the loop occupying additional space on the outer surface of the active site. The binuclear active site manganese ions (Mn) are shown.

FIG. 4. Size and shape comparison of an hArg1:mAb4 2:3 trimer complex. Left panel: The mAb4 is approximately 195 A in length when measuring between the C-α of the Arg222 residues in both the top and bottom B monomers of the hArg1 trimer. The three monomers of the hArg1 trimer are shown as monomers A, B, and C, and the mAb4 heavy chain and light chain are colored dark grey and light grey, respectively. Shown are the Fab1 and Fab2 of each of three mAb4 antibodies (Fab, Fab′, and Fab″). Right panel: the binding of Fab1 of mAb4 to monomers A and B of an hArg1 trimer is depicted in cartoon form and illustrates how Fab1 binds to monomers A and B.

FIG. 5. Predominant N-linked glycans for monoclonal antibodies produced in Chinese hamster ovary cells (CHO N-linked glycans) and in engineered yeast cells (engineered yeast N-linked glycans): squares: N-acetylglucosamine (GlcNac); circles: mannose (Man); diamonds: galactose (Gal); triangles: fucose (Fuc).

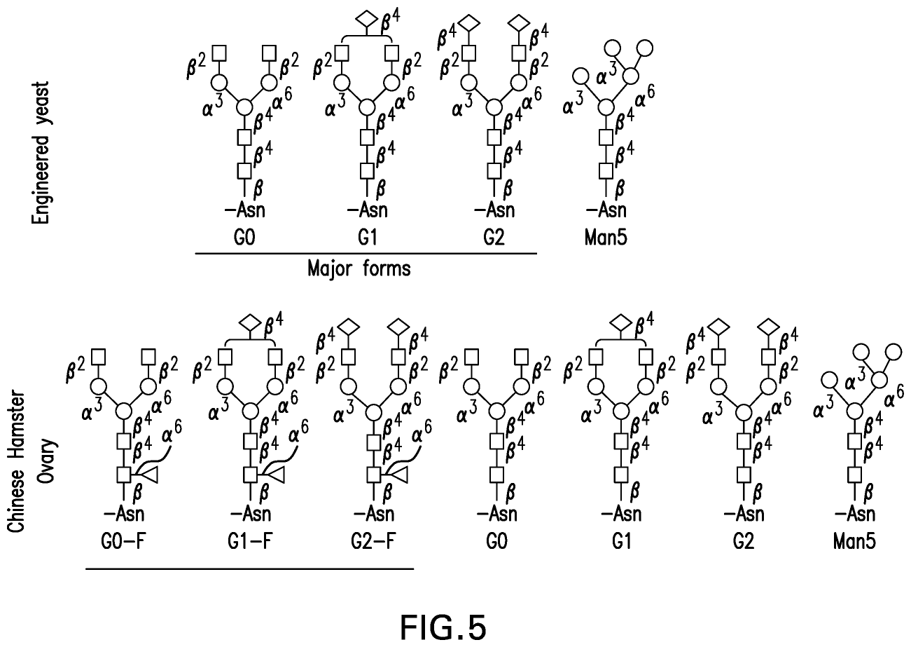

FIG. 6. Shows the amino acid sequence for human Arginase 1 (SEQ ID NO: 1). The amino acids boxed are amino acids at the mouth of the active site and the amino acids underlined are those amino acids involved in monomer:monomer interactions.

### DETAILED DESCRIPTION OF THE INVENTION

### Definitions

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

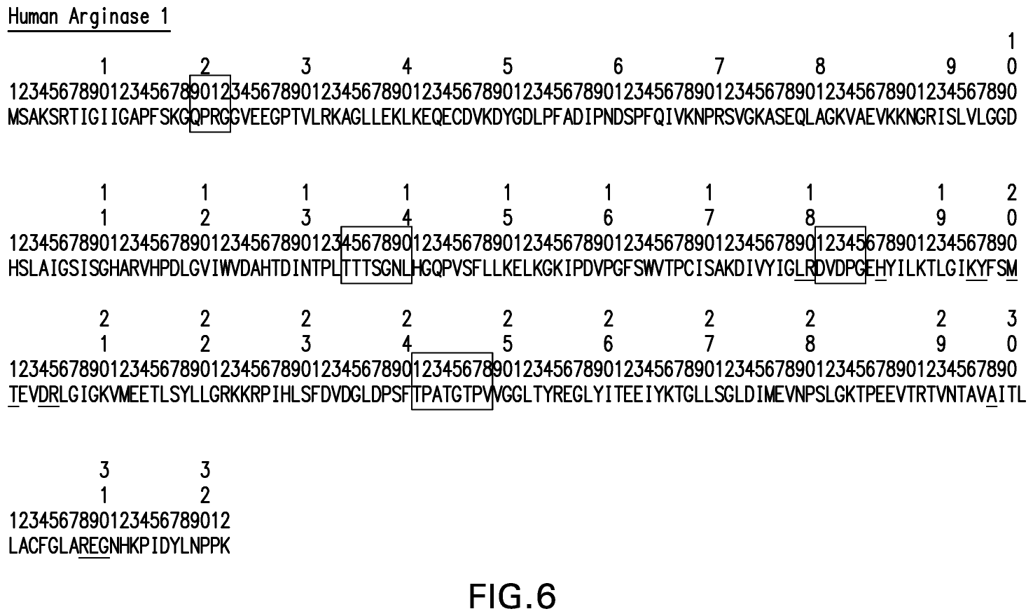

As used herein, the term “Arginase” refers to a manganese-containing enzyme (EC 3.5.3.1, arginine amidinase, canavanase, L-arginase, arginine transamidinase) that catalyzes the conversion of arginine to ornithine+urea. It is the final enzyme of the urea cycle and is ubiquitous to all domains of life. Two isozymes of this arginase exist: arginase 1, which functions in the urea cycle, and is located primarily in the cytoplasm of hepatocytes (liver cells); and arginase 2, which may be involved in the regulation of intracellular arginine/ornithine levels, and is located in mitochondria of several tissues in the body, with most abundance in the kidney and prostate and at lower levels in macrophages, lactating mammary glands, and brain. The amino acid sequence of human Arginase 1 is set forth in SEQ ID NO: 1 and shown in FIG. 6.

As used herein, the term “hArg1” refers to human arginase 1.

As used herein, the term “Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (KD). Affinity can be measured by common methods known in the art, including KinExA and Biacore. Specific illustrative and exemplary embodiments for measuring binding affinity are described in the following.

As used herein, the term “administration” and “treatment,” as it applies to an animal, human, experimental subject, cell, tissue, organ, or biological fluid, refers to contact of an exogenous pharmaceutical, therapeutic, diagnostic agent, or composition comprising an Arginase 1 binder as disclosed herein to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., human, rat, mouse, dog, cat, rabbit). In a preferred embodiment, the term “subject” refers to a human.

As used herein, the term “amino acid” refers to a simple organic compound containing both a carboxyl (—COOH) and an amino (—NH₂) group. Amino acids are the building blocks for proteins, polypeptides, and peptides. Amino acids occur in L-form and D-form, with the L-form in naturally occurring proteins, polypeptides, and peptides. Amino acids and their code names are set forth in the following chart.

| | Three letter | One letter |
|---|---|---|
| Amino acid | code | code |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “antibody” or “immunoglobulin” as used herein refers to a glycoprotein comprising either (a) at least two heavy chains (HCs) and two light chains (LCs) inter-connected by disulfide bonds, or (b) in the case of a species of camelid antibody, at least two heavy chains (HCs) inter-connected by disulfide bonds. Each HC is comprised of a heavy chain variable region or domain (VH) and a heavy chain constant region or domain. Each light chain is comprised of an LC variable region or domain (VL) and a LC constant domain. In certain naturally occurring IgG, IgD and IgA antibodies, the heavy chain constant region is comprised of three domains, CH1, CH2 and CH3. In general, the basic antibody structural unit for antibodies is a Y-shaped tetramer comprising two HC/LC pairs (2H+2L), except for the species of camelid antibodies comprising only two HCs (2H), in which case the structural unit is a homodimer. Each tetramer includes two identical pairs of polypeptide chains, each pair having one LC (about 25 kDa) and HC chain (about 50-70 kDa) (H+L). Each HC:LC pair comprises one VH: one VL pair. The one VH:one VL pair may be referred to by the term “Fab”. Thus, each antibody tetramer comprises two Fabs, one per each arm of the Y-shaped antibody.

The LC constant domain is comprised of one domain, CL. The human VH includes seven family members: VH1, VH2, VH3, VH4, VHS, VH6, and VH7; and the human VL includes 16 family members: Vκ1, Vκ2, Vκ3, Vκ4, Vκ5, Vκ6, Vλ1, Vλ2, Vλ3, Vλ4, Vλ5, Vλ6, Vλ7, Vλ8, Vλ9, and Vλ10. Each of these family members can be further divided into particular subtypes. The VH and VL can be further subdivided into regions of hypervariability, termed complementarity determining region (CDR) areas, interspersed with regions that are more conserved, termed framework regions (FR). Each VH and VL is composed of three CDR regions and four FR regions, arranged from amino-terminus to carboxy-terminus in the following order: FR1, CDR1, FR2, CDR2, FR3, CDR3, FR4. Numbering of the amino acids in a VH or VHH may be determined using the Kabat numbering scheme. See Béranger, et al., Ed. Ginetoux, Correspondence between the IMGT unique numbering for C-DOMAIN the IMGT exon numbering, the Eu and Kabat numberings: Human IGHG, Created: May 17, 2001, Version: Jun. 8, 2016, which is accessible at www.imgt.org/IMGTScientificChart/Numbering/Hu_IGHGnber.html).

The constant regions of the antibodies may mediate the binding of the immunoglobulin to host tissues or factors, including various cells of the immune system (e.g., effector cells) and the first component (C1q) of the classical complement system. Typically, the numbering of the amino acids in the heavy chain constant domain begins with number 118, which is in accordance with the Eu numbering scheme. The Eu numbering scheme is based upon the amino acid sequence of human IgG1 (Eu), which has a constant domain that begins at amino acid position 118 of the amino acid sequence of the IgG1 described in Edelman et al., Proc. Natl. Acad. Sci. USA. 63: 78-85 (1969), and is shown for the IgG1, IgG2, IgG3, and IgG4 constant domains in Beranger, et al., op. cit.

The variable regions of the heavy and light chains contain a binding domain comprising the CDRs that interacts with an antigen. A number of methods are available in the art for defining CDR sequences of antibody variable domains (see Dondelinger et al., Frontiers in Immunol. 9: Article 2278 (2018)). The common numbering schemes include the following.
Kabat numbering scheme is based on sequence variability and is the most commonly used (See Kabat et al. Sequences of Proteins of Immunological Interest, 5th Ed. Public Health Service, National Institutes of Health, Bethesda, Md. (1991) (defining the CDR regions of an antibody by sequence);
Chothia numbering scheme is based on the location of the structural loop region (See Chothia & Lesk J. Mol. Biol. 196: 901-917 (1987); Al-Lazikani et al., J. Mol. Biol. 273: 927-948 (1997));
AbM numbering scheme is a compromise between the two used by Oxford Molecular's AbM antibody modelling software (see Karu et al, ILAR Journal 37: 132-141 (1995);
Contact numbering scheme is based on an analysis of the available complex crystal structures (See www.bioinf.org.uk: Prof Andrew C. R. Martin's Group; Abhinandan & Martin, Mol. Immunol. 45:3832-3839 (2008)).
IMGT (ImMunoGeneTics) numbering scheme is a standardized numbering system for all the protein sequences of the immunoglobulin superfamily, including variable domains from antibody light and heavy chains as well as T cell receptor chains from different species and counts residues continuously from 1 to 128 based on the germ-line V sequence alignment (see Giudicelli et al., Nucleic Acids Res. 25:206-11 (1997); Lefranc, Immunol Today 18:509(1997); Lefranc et al., Dev Comp Immunol. 27:55-77 (2003)).

The following general rules disclosed in www.bioinforg.uk: Prof. Andrew C. R. Martin's Group and reproduced in Table 1 below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

TABLE 1

| Loop | Kabat | AbM | Chothia¹ | Contact² | IMGT |
|---|---|---|---|---|---|
| L1 | L24--L34 | L24--L34 | L24--L34 | L30--L36 | L27--L32 |
| L2 | L50--L56 | L50--L56 | L50--L56 | L46--L55 | L50--L51 |
| L3 | L89--L97 | L89--L97 | L89--L97 | L89--L96 | L89--L97 |
| H1 | H31--H35B | H26--H35B | H26--H32 . . . 34 | H30--H35B | H26--H35B |
| | (Kabat Numbering)³ | | | | |
| H1 | H31--H35 | H26--H35 | H26--H32 | H30--H35 | H26--H33 |
| | (Chothia Numbering) | | | | |
| H2 | H50--H65 | H50--H58 | H52--H56 | H47--H58 | H51--H56 |
| H3 | H95--H102 | H95--H102 | H95--H102 | H93--H101 | H93--H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

The entire nucleotide sequence of the heavy chain and light chain variable regions are commonly numbered according to Kabat while the three CDRs within the variable region may be defined according to any one of the aforementioned numbering schemes.

In general, the state of the art recognizes that in many cases, the CDR3 region of the heavy chain is the primary determinant of antibody specificity, and examples of specific antibody generation based on CDR3 of the heavy chain alone are known in the art (e.g., Beiboer et al., J. Mol. Biol. 296: 833-849 (2000); Klimka et al., British J. Cancer 83: 252-260 (2000); Rader et al., Proc. Natl. Acad. Sci. USA 95: 8910-8915 (1998); Xu et al., Immunity 13: 37-45 (2000).

As used herein, the term “Fc domain”, or “Fc” as used herein is the crystallizable fragment domain or region obtained from an antibody that comprises the CH2 and CH3 domains of an antibody. In an antibody, the two Fc domains are held together by two or more disulfide bonds and by hydrophobic interactions of the CH3 domains. The Fc domain may be obtained by digesting an antibody with the protease papain. Typically, amino acids in the Fc domain are numbered according to the Eu numbering convention (See Edelmann et al., Biochem. 63: 78-85 (1969)).

As used herein, the term “antigen” as used herein refers to any foreign substance which induces an immune response in the body.

As used here, the term “Arginase 1 binder” refers to a polypeptide or protein that binds to arginase 1. An Arginase 1 binder includes but is not limited to a bivalent antibody tetramer (2H+2L), a monovalent antibody (H+L), a bi-specific antibody that targets arginase 1 and another target, a Fab fragment, a Fab′ fragment, a F(ab′)2 fragment, an Fv region, and an ScFv. Unless otherwise indicated, the Arginase 1 binders herein bind to and inhibit the activity of hARG1.

As used herein, the term “antigen binding fragment” refers to a polypeptide or polypeptides comprising a fragment of a full length antibody, which retains the ability to specifically bind to the antigen bound by the full length antibody, and/or to compete with the full length antibody for specifically binding to the antigen. Examples of antigen binding fragments include but are not limited to Fab fragment, Fab′ fragment, F(ab′)2 fragment, Fv region, and scFv.

As used herein, the term “Fab fragment” refers to an antigen binder comprising one antibody light chain and the CH1 and VH of one antibody heavy chain. The heavy chain of a Fab molecule cannot form a disulfide bond with another heavy chain molecule. A “Fab fragment” can be the product of papain cleavage of an antibody.

As used herein, the term “Fab′ fragment” refers to an antigen binder comprising one antibody light chain and a portion or fragment of one antibody heavy chain that contains the VH and the CH1 domain up to a region between the CH1 and CH2 domains, such that an interchain disulfide bond can be formed between the two heavy chains of two Fab′ fragments to form a F(ab′)2 molecule.

As used herein, the term “F(Ab′)2 fragment” refers to an antigen binder comprising two antibody light chains and two heavy chains containing the VH and the CH1 domain up to a region between the CH1 and CH2 domains, such that an interchain disulfide bond is formed between the two heavy chains. An F(Ab′)2 fragment thus is composed of two FAb′ fragments that are held together by a disulfide bond between the two heavy chains. An “F(Ab′)2 fragment” can be the product of pepsin cleavage of an antibody.

As used herein, the term “Fv region” refers to an antigen binder comprising the variable regions from both the heavy and light chains of an antibody, but lacks the constant regions.

As used herein, the term “ScFv” or “single-chain variable fragment” refers to a fusion protein comprising a VH and VL fused or linked together by a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker.

As used herein, the term “diabody” refers to an antigen binder comprising a small antibody fragment with two antigen-binding regions, which fragments comprise a heavy chain variable domain (VH) connected to a light chain variable domain (VL) in the same polypeptide chain (VH-VL or VL-VH). By using a linker that is too short to allow pairing between the two domains on the same chain, the domains are forced to pair with the complementarity domains of another chain and create two antigen-binding regions. Diabodies are described more fully in, e.g., EP 404,097; WO 93/11161; and Holliger et al. (1993) Proc. Natl. Acad. Sci. USA 90: 6444-6448. For a review of engineered antibody variants generally see Holliger and Hudson (2005) Nat. Biotechnol. 23:1126-1136.

These and other potential constructs are described at Chan & Carter (2010) Nat. Rev. Immunol. 10:301. These antibody fragments are obtained using conventional techniques known to those with skill in the art, and the fragments are screened for utility in the same manner as are intact antibodies. Antigen-binding fragments can be produced by recombinant DNA techniques, or by enzymatic or chemical cleavage of intact immunoglobulins.

As used herein, the term “isolated” antibodies or antigen-binding fragments thereof (e.g., antigen binders such as Arginase 1 binders) are at least partially free of other biological molecules from the cells or cell cultures in which they are produced. Such biological molecules include nucleic acids, proteins, lipids, carbohydrates, or other material such as cellular debris and growth medium. An isolated antibody or antigen-binding fragment may further be at least partially free of expression system components such as biological molecules from a host cell or of the growth medium thereof. Generally, the term “isolated” is not intended to refer to a complete absence of such biological molecules or to an absence of water, buffers, or salts or to components of a pharmaceutical formulation that includes the antibodies or fragments.

As used herein, the term “monoclonal antibody” refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains that are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, the term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters. Genes include both naturally occurring nucleotide sequences encoding a molecule of interest and synthetically derived nucleotide sequences encoding a molecule of interest, for example, complementary DNA (cDNA) obtained from a messenger RNA (mRNA) nucleotide sequence.

As used herein, the term “germline” or “germline sequence” refers to a sequence of unrearranged immunoglobulin DNA sequences. Any suitable source of unrearranged immunoglobulin sequences may be used. Human germline sequences may be obtained, for example, from JOINSOLVER® germline databases on the website for the National Institute of Arthritis and Musculoskeletal and Skin Diseases of the United States National Institutes of Health. Mouse germline sequences may be obtained, for example, as described in Giudicelli et al. (2005) Nucleic Acids Res. 33:D256-D261.

As used herein, the term “library” as used herein is, typically, a collection of related but diverse polynucleotides that are, in general, in a common vector backbone. For example, a light chain or heavy chain immunoglobulin library may contain polynucleotides, in a common vector backbone, that encode light and/or heavy chain immunoglobulins, which are diverse but related in their nucleotide sequence; for example, which immunoglobulins are functionally diverse in their abilities to form complexes with other immunoglobulins, e.g., in an antibody display system of the present invention, and bind a particular antigen.

As used herein, the term “polynucleotides” discussed herein form part of the present invention. A “polynucleotide”, “nucleic acid ” or “nucleic acid molecule” include DNA and RNA, single-or double-stranded. Polynucleotides e.g., encoding an immunoglobulin chain or component of the antibody display system of the present invention, may, in an embodiment of the invention, be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′-and 3′-non-coding regions, and the like.

Polynucleotides e.g., encoding an immunoglobulin chain or component of the antibody display system of the present invention, may be operably associated with a promoter. A “promoter” or “promoter sequence” is, in an embodiment of the invention, a DNA regulatory region capable of binding an RNA polymerase in a cell (e.g., directly or through other promoter-bound proteins or substances) and initiating transcription of a coding sequence. A promoter sequence is, in general, bounded at its 3′ terminus by the transcription initiation site and extends upstream (5′ direction) to include the minimum number of bases or elements necessary to initiate transcription at any level. Within the promoter sequence may be found a transcription initiation site (conveniently defined, for example, by mapping with nuclease S1), as well as protein binding domains (consensus sequences) responsible for the binding of RNA polymerase. The promoter may be operably associated with other expression control sequences, including enhancer and repressor sequences or with a nucleic acid of the invention. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the β-lactamase promoter (Villa-Komaroff, et al., (1978) Proc. Natl. Acad. Sci. USA 75:3727-3731), or the tac promoter (DeBoer, et al., (1983) Proc. Natl. Acad. Sci. USA see also “Useful proteins from recombinant bacteria” in Scientific American (1980) 242:74-94; and promoter elements from yeast or other fungi such as the Gal 4 promoter, the ADC (alcohol dehydrogenase) promoter, PGK (phosphoglycerol kinase) promoter or the alkaline phosphatase promoter.

As used herein, the terms “vector”, “cloning vector” and “expression vector” include a vehicle (e.g., a plasmid) by which a DNA or RNA sequence can be introduced into a host cell, so as to transform the host and, optionally, promote expression and/or replication of the introduced sequence. Polynucleotides encoding an immunoglobulin chain or component of the antibody display system of the present invention may, in an embodiment of the invention, be in a vector.

As used herein, the terms “cell,” “cell line,” and “cell culture” are used interchangeably and all such designations include progeny. Thus, the words “transformants” and “transformed cells” include the primary subject cell and cultures derived therefrom without regard for the number of transfers. It is also understood that not all progeny will have precisely identical DNA content, due to deliberate or inadvertent mutations. Mutant progeny that have the same function or biological activity as screened for in the originally transformed cell are included. Where distinct designations are intended, it will be clear from the context.

As used herein, the term “control sequences” or “regulatory sequences” refers to DNA sequences necessary for the expression of an operably linked coding sequence in a particular host organism. The control sequences that are suitable for expression in eukaryotes, for example, include a promoter, operator or enhancer sequences, transcription termination sequences, and polyadenylation sequences for expression of a messenger RNA encoding a protein and a ribosome binding site for facilitating translation of the messenger RNA.

As used herein, a nucleic acid is “operably linked” when it is placed into a functional relationship with another nucleic acid sequence, e.g., a regulatory sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

As used herein, the term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “expression” as used herein is defined as the transcription and/or translation of a particular nucleotide sequence.

As used herein, the term “inhibits arginase 1 activity” means the ability to inhibit the formation of ornithine from arginine. Inhibition may be determined in an assay that detects formation of thioornithine from thioarginine, for example, the assay taught in Example 4 or by liquid chromatography-mass spectroscopy (LC-MS), for example, as taught in Example 5.

As used herein, “treat” or “treating” means to administer a therapeutic agent, such as a composition containing any of the Arginase binders of the present invention, topically, subcutaneously, intramuscular, intradermally, or systemically to an individual in need. The amount of a therapeutic agent that is effective to alleviate cancer or proliferative disease in the individual may vary according to factors such as the injury or disease state, age, and/or weight of the individual, and the ability of the therapeutic agent to elicit a desired response in the individual. Whether the therapeutic objective has been achieved can be assessed by the individual and/or any clinical measurement typically used by physicians or other skilled healthcare providers to assess the severity or progression status of the treatment. Thus, the terms denote that a beneficial result has been or will be conferred on a human or animal individual in need.

As used herein, “treatment,” as it applies to a human or veterinary individual, refers to therapeutic treatment, as well as diagnostic applications. “Treatment” as it applies to a human or veterinary individual, encompasses contact of the antibodies or antigen binding fragments of the present invention to a human or animal subject.

As used herein, “therapeutically effective amount” refers to a quantity of a specific substance sufficient to achieve a desired effect in an individual being treated. For instance, this may be the amount necessary to inhibit or reduce the severity of cancer or proliferative disease in an individual.

As used herein, the term “Combination therapy” refers to treatment of a human or animal individual comprising administering a first therapeutic agent and a second therapeutic agent consecutively or concurrently to the individual. In general, the first and second therapeutic agents are administered to the individual separately and not as a mixture; however, there may be embodiments where the first and second therapeutic agents are mixed prior to administration.

### Arginase 1 Binders

The Arginase 1 binders of the present invention are humanized antibodies obtained from mouse hybridoma obtained from a mouse immunized with hArg1. The Arginase 1 binders of the present invention comprise VH and VL derived from hybridoma antibody mAb4. The mouse mAb4 VH and VL (designated VH1 and VL1 domains, respectively) were humanized to produce a humanized mAb4 comprising humanized VH and VL (designated VH2 and VL2, respectively).

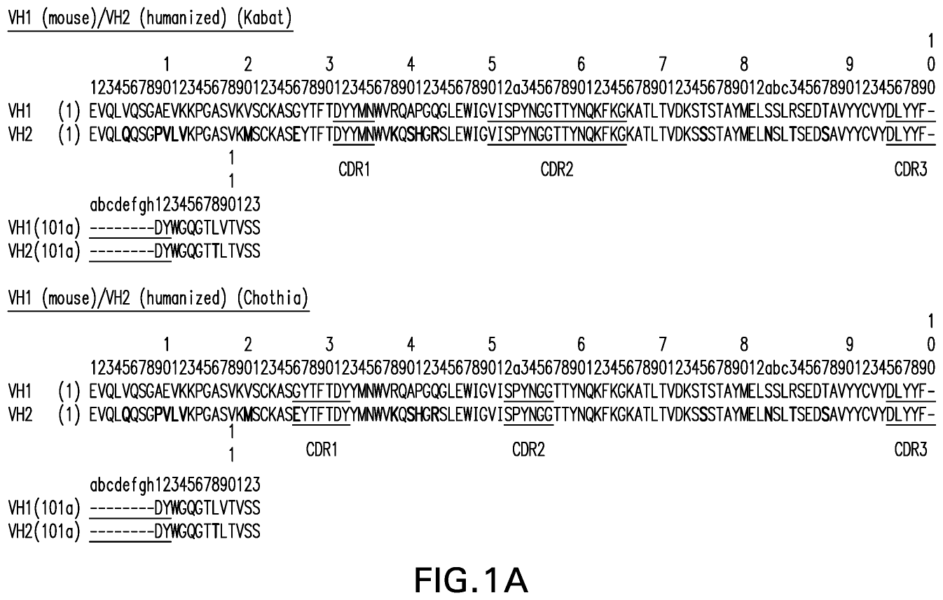

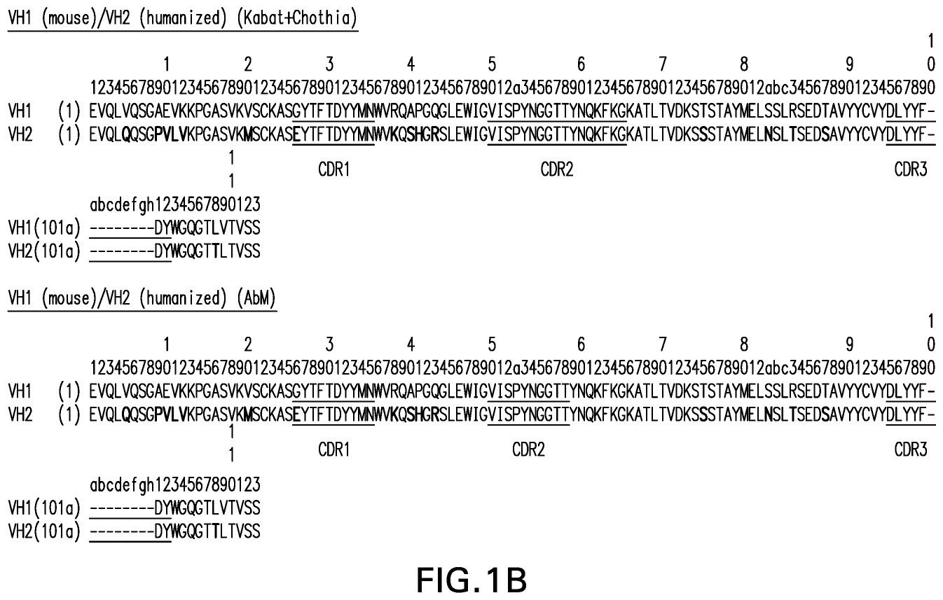

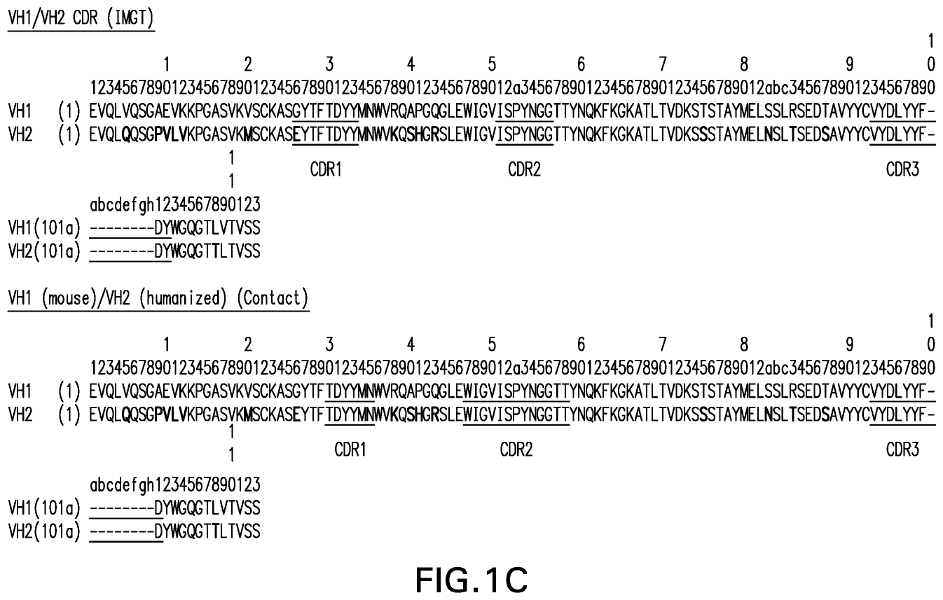

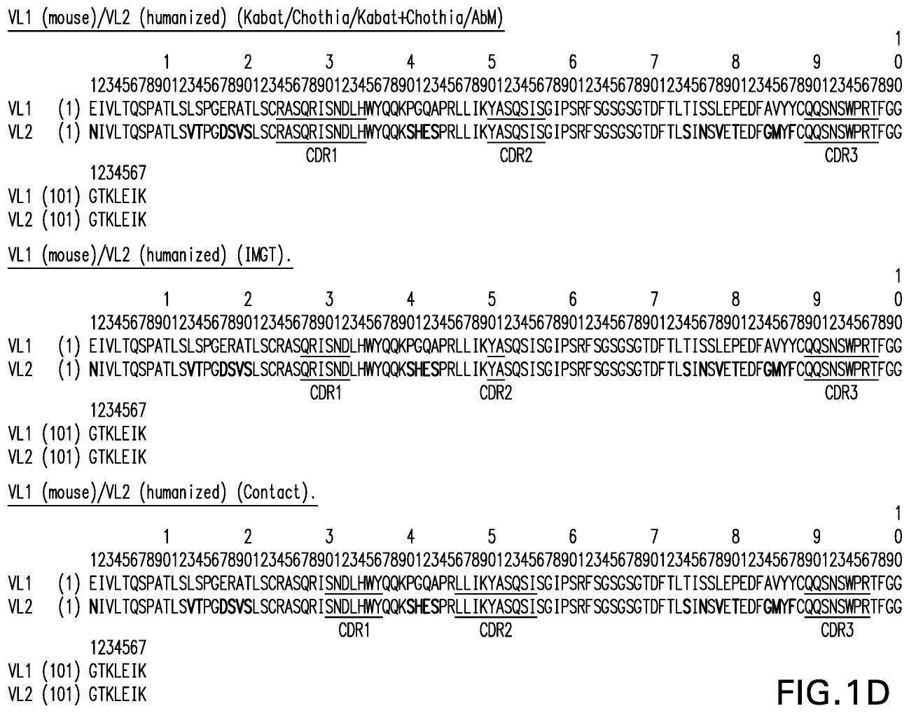

These Arginase 1 binders of the present invention comprise six complementarity determining regions (CDRs) comprising a particular combination of three CDRs as presented in the table below. The CDR amino acid sequences shown in Tables 2-7 and FIGS. 1A-1D are set forth according to the Kabat, Chothia, Kabat+Chothia, AbM, IMGT, and Contact numbering schemes for identifying CDR amino acid sequences. A particular CDR amino acid sequence determined using any one of the these schemes advanced for identifying CDR amino acid sequences (See Table 1) have more or less amino acids than that of CDR amino acid sequences identified according to any other numbering scheme but will overlap the CDR amino acid sequences. Thus, the CDR amino acid sequences shown herein are not to be construed as limiting and any Arginase 1 binder in which the CDR amino acid sequences have been identified by another numbering scheme will fall within the scope of the Arginase 1 binders of the present invention provided the amino acid sequences for such Arginase 1 binders comprise the six CDR amino acid sequences as identified by any one of the numbering schemes and shown in Tables 2-7 and shown FIGS. 1A-1D. For all antibodies disclosed herein unless indicated otherwise, the amino acids comprising the variable domains are numbered according to the Kabat numbering scheme independently of how the amino acids are defined using Kabat, Chothia, Kabat+Chothia, AbM, IMGT, or Contact schemes and the constant domains are numbered according to the Eu numbering scheme.

The Arginase 1 binders comprise a VH1 or VH2 and a VL, each domain comprising three CDRs and four Frameworks (FR) in the following arrangement
FR1-CDR1-FR2-CDR2-FR3-CDR3-FR4.

The Arginase 1 binder mouse VH1-CDRs and VL1-CDRs and humanized VH2-CDRs and VL2-CDRs may comprise the amino acid sequences shown in Table 2-7 as defined according to

Kabat, Chothia, Kabat+Chothia, AbM, IMGT, and Contact numbering schemes, respectively. As shown in the tables, an E amino acid substitution in FR1 adjacent to the VH2-CDR1 for CDRs defined by Kabat or Contact is at the N-terminus of the VH2-CDR1 amino acid sequence when the CDRs are defined by Chothia, Kabat+Chothia, AbM, and IMGT.

TABLE 2
Amino Acid Sequences of Arginase 1 Binder CDRs
as defined by Kabat

| | VH1-CDR1/ | VH1-CDR2/ | VH1-CDR3/ |
|---|---|---|---|
| | VH2-CDR1 | VH-CDR2 | VH-CDR3 |
| VH1/ | DYYMN | VISPYNGNGTTYNQKFGK | DLYYFDY |
| VH2 | (SEQ ID NO: 6) | (SEQ ID NO: 7) | (SEQ ID NO: 8) |
| | VL1-CDR1/ | VL1-CDR2/ | VL1-CDR3/ |
| | VL2-CDR1 | VL2-CDR2 | VL2-CDR3 |
| VL1/ | RASQRISNDLH | YASQSIS | QQSNSWPRT |
| VL2 | (SEQ ID NO: 22) | (SEQ ID NO: 23) | (SEQ ID NO: 24) |

TABLE 3
Amino Acid Sequences of Arginase 1 Binder CDRs
as defined by Chothia

| | VH1-CDR1 | VH1-CDR2 | VH1-CDR3 |
|---|---|---|---|
| VH1 | GYTFTDY | SPYNGG | DLYYFDY |
| | (SEQ ID NO: 9) | (SEQ ID NO: 11) | (SEQ ID NO: 8) |
| | VH2-CDR1 | VH2-CDR2 | VH2-CDR3 |
| VH2 | EYTFTDY | SPYNGG | DLYYFDY |
| | (SEQ ID N: 10) | (SEQ ID NO: 11) | (SEQ ID NO: 8) |
| | VL1-CDR1/ | VL1-CDR2/ | VL1-CDR3/ |
| | VL2-CDR1 | VL2-CDR2 | VL2-CDR3 |
| VL1/ | RASQRISNDLH | YASQSIS | QQSNSWPRT |
| VL2 | (SEQ ID NO: 22) | (SEQ ID NO: 23) | (SEQ ID NO: 24) |

TABLE 4
Amino Acid Sequences of Arginase 1 Binder CDRs
as defined by Kabat + Chothia

| | VH1-CDR1 | VH1-CDR2 | VH1-CDR3 |
|---|---|---|---|
| VH1 | GYTFTDYYMN | VISPYNGGTTYNQKFGK | DLYYFDY |
| | (SEQ ID NO: 12) | (SEQ ID NO: 7) | (SEQ ID NO: 8) |
| | VH2-CDR1 | VH2-CDR2 | VH2-CDR3 |
| VH2 | EYTFTDYYMN | VISPYNGGTTYNQKFGK | DLYYFDY |
| | (SEQ ID NO: 13) | (SEQ ID NO: 7) | (SEQ ID NO: 8) |
| | VL1-CDR1/ | VL1-CDR2/ | VL1-CDR3/ |
| | VL2-CDR1 | VL2-CDR2 | VL2-CDR3 |
| VL1/ | RASQRISNDLH | YASQSIS | QQSNSWPRT |
| VL2 | (SEQ ID NO: 22) | (SEQ ID NO: 23) | (SEQ ID NO: 24) |

TABLE 5
Amino Acid Sequences of Arginase 1 Binder CDRs
as defined by AbM

| | VH1-CDR1 | VH1-CDR2 | VH1-CDR3 |
|---|---|---|---|
| VH1 | GYTFTDYYMN | VISPYNGGTT | DLYYFDY |
| | (SEQ ID NO: 12) | (SEQ ID NO: 14) | (SEQ ID NO: 8) |
| | VH2-CDR1 | VH2-CDR2 | VH2-CDR3 |
| VH2 | EYTFTDYYMN | VISPYNGGTT | DLYYFDY |
| | (SEQ ID NO: 13) | (SEQ ID NO: 14) | (SEQ ID NO: 8) |
| | VL1-CDR1/ | VL1-CDR2/ | VL1-CDR3/ |
| | VL2-CDR1 | VL2-CDR2 | VL2-CDR3 |
| VL1/ | RASQRISNDLH | YASQSIS | QQSNSWPRT |
| VL2 | (SEQ ID NO: 22) | (SEQ ID NO: 23) | (SEQ ID NO: 24) |

TABLE 6
Amino Acid Sequences of Arginase 1 Binder CDRs
as defined by IMGT

| | VH1-CDR1 | VH1-CDR2 | VH1-CDR3 |
|---|---|---|---|
| VH1 | GYTFTDYY | ISPYNGG | VYDLYYFDY |
| | (SEQ ID NO: 15) | (SEQ ID NO: 17) | (SEQ ID NO: 18) |
| | VH2-CDR1 | VH2-CDR2 | VH2-CDR3 |
| VH2 | EYTFTDYY | ISPYNGG | VYDLYYFDY |
| | (SEQ ID NO: 16) | (SEQ ID NO: 17) | (SEQ ID NO: 18) |
| | VL1-CDR1/ | VL1-CDR2/ | VL1-CDR3/ |
| | VL2-CDR1 | VL2-CDR2 | VL2-CDR3 |
| VL1/ | QRISND | AA | QQSNSWPRT |
| VL2 | (SEQ ID NO: 25) | | (SEQ ID NO: 24) |

TABLE 7
Amino Acid Sequences of Arginase 1 Binder CDRs
as defined by Contact

| | VH1-CDR1/ | VH1-CDR2/ | VH1-CDR3/ |
|---|---|---|---|
| | VH2-CDR1 | VH-CDR2 | VH-CDR3 |
| VH1/ | TDYYMN | WIGVISPYNGGTT | VYDLYYFD |
| VH2 | (SEQ ID NO: 19) | (SEQ ID NO: 20) | (SEQ ID NO: 21) |
| | VL1-CDR1/ | VL1-CDR2/ | VL1-CDR3/ |
| | VL2-CDR1 | VL2-CDR2 | VL2-CDR3 |
| VL1/ | SNDLHWY | LLIKYASQSI | QQSNSWPR |
| VL2 | (SEQ ID NO: 26) | (SEQ ID NO: 27) | (SEQ ID NO: 28) |

In particular embodiments of the invention, the Arginase 1 binder comprises (a) a VH1-CDR1 or VH2-CDR1 comprising the amino acid sequence set forth in SEQ ID NO: 6, a VH1-CDR2 or VH2-CDR2 comprising the amino acid sequence set forth in SEQ ID NO: 7, and a VH1-CDR3 or VH2-CDR3 comprising the amino acid sequence set forth in SEQ ID NO: 8; and (b) a VL1-CDR1 or VL2-CDR1 comprising the amino acid sequence set forth in SEQ ID NO: 22, a VL1-CDR2 or VL2-CDR2 comprising the amino acid sequence set forth in SEQ ID NO: 23, and a VL1-CDR3 or VL2-CDR3 comprising the amino acid sequence set forth in SEQ ID NO: 24. The Arginase 1 binders specifically bind arginase 1 and inhibit arginase 1 activity. CDR sequences are determined using the Kabat numbering scheme.

In particular embodiments of the invention, the Arginase 1 binder comprises (a) a VH1-CDR1 comprising the amino acid sequence set forth in SEQ ID NO: 9, a VH1-CDR2 comprising the amino acid sequence set forth in SEQ ID NO: 11, and a VH1-CDR3 comprising the amino acid sequence set forth in SEQ ID NO: 8; and (b) a VL1-CDR1 or VL2-CDR1 comprising the amino acid sequence set forth in SEQ ID NO: 22, a VL1-CDR2 or VL2-CDR2 comprising the amino acid sequence set forth in SEQ ID NO: 23, and a VL1-CDR3 or VL2-CDR3 comprising the amino acid sequence set forth in SEQ ID NO: 24. The Arginase 1 binders specifically bind arginase 1 and inhibit arginase 1 activity. CDR sequences are determined using the Chothia numbering scheme.

In particular embodiments of the invention, the Arginase 1 binder comprises (a) a VH2-CDR1 comprising the amino acid sequence set forth in SEQ ID NO: 10, a VH2-CDR2 comprising the amino acid sequence set forth in SEQ ID NO: 11, and a VH2-CDR3 comprising the amino acid sequence set forth in SEQ ID NO: 8; and (b) a VL1-CDR1 or VL2-CDR1 comprising the amino acid sequence set forth in SEQ ID NO: 22, a VL1-CDR2 or VL2-CDR2 comprising the amino acid sequence set forth in SEQ ID NO: 23, and a VL1-CDR3 or VL2-CDR3 comprising the amino acid sequence set forth in SEQ ID NO: 24. The Arginase 1 binders specifically bind arginase 1 and inhibit arginase 1 activity. CDR sequences are determined using the Chothia numbering scheme.

In particular embodiments of the invention, the Arginase 1 binder comprises (a) a VH1-CDR1 comprising the amino acid sequence set forth in SEQ ID NO: 12, a VH1-CDR2 comprising the amino acid sequence set forth in SEQ ID NO: 7, and a VH1-CDR3 comprising the amino acid sequence set forth in SEQ ID NO: 8; and (b) a VL1-CDR1 or VL2-CDR1 comprising the amino acid sequence set forth in SEQ ID NO: 22, a VL1-CDR2 or VL2-CDR2 comprising the amino acid sequence set forth in SEQ ID NO: 23, and a VL1-CDR3 or VL2-CDR3 comprising the amino acid sequence set forth in SEQ ID NO: 24. The Arginase 1 binders specifically bind arginase 1 and inhibit arginase 1 activity. CDR sequences are determined using the Kabat+Chothia numbering scheme.

In particular embodiments of the invention, the Arginase 1 binder comprises (a) a VH2-CDR1 comprising the amino acid sequence set forth in SEQ ID NO: 13, a VH2-CDR2 comprising the amino acid sequence set forth in SEQ ID NO: 7, and a VH2-CDR3 comprising the amino acid sequence set forth in SEQ ID NO: 8; and (b) a VL1-CDR1 or VL2-CDR1 comprising the amino acid sequence set forth in SEQ ID NO: 22, a VL1-CDR2 or VL2-CDR2 comprising the amino acid sequence set forth in SEQ ID NO: 23, and a VL1-CDR3 or VL2-CDR3 comprising the amino acid sequence set forth in SEQ ID NO: 24. The Arginase 1 binders specifically bind arginase 1 and inhibit arginase 1 activity. CDR sequences are determined using the Kabat+Chothia numbering scheme.

In particular embodiments of the invention, the Arginase 1 binder comprises (a) a VH1-CDR1 comprising the amino acid sequence set forth in SEQ ID NO: 12, a VH1-CDR2 comprising the amino acid sequence set forth in SEQ ID NO: 14, and a VH1-CDR3 comprising the amino acid sequence set forth in SEQ ID NO: 8; and (b) a VL1-CDR1 or VL2-CDR1 comprising the amino acid sequence set forth in SEQ ID NO: 22, a VL1-CDR2 or VL2-CDR2 comprising the amino acid sequence set forth in SEQ ID NO: 23, and a VL1-CDR3 or VL2-CDR3 comprising the amino acid sequence set forth in SEQ ID NO: 24. The Arginase 1 binders specifically bind arginase 1 and inhibit arginase 1 activity. CDR sequences are determined using the AbM numbering scheme.

In particular embodiments of the invention, the Arginase 1 binder comprises (a) a VH2-CDR1 comprising the amino acid sequence set forth in SEQ ID NO: 13, a VH2-CDR2 comprising the amino acid sequence set forth in SEQ ID NO: 14, and a VH2-CDR3 comprising the amino acid sequence set forth in SEQ ID NO: 8; and (b) a VL1-CDR1 or VL2-CDR1 comprising the amino acid sequence set forth in SEQ ID NO: 22, a VL1-CDR2 or VL2-CDR2 comprising the amino acid sequence set forth in SEQ ID NO: 23, and a VL1-CDR3 or VL2-CDR3 comprising the amino acid sequence set forth in SEQ ID NO: 24. The Arginase 1 binders specifically bind arginase 1 and inhibit arginase 1 activity. CDR sequences are determined using the AbM numbering scheme.

In particular embodiments of the invention, the Arginase 1 binder comprises (a) a VH1-CDR1 comprising the amino acid sequence set forth in SEQ ID NO: 15, a VH1-CDR2 comprising the amino acid sequence set forth in SEQ ID NO: 17, and a VH1-CDR3 comprising the amino acid sequence set forth in SEQ ID NO: 18; and (b) a VL1-CDR1 or VL2-CDR1 comprising the amino acid sequence set forth in SEQ ID NO: 25, a VL1-CDR2 or VL2-CDR2 comprising the amino acid sequence AA, and a VL1-CDR3 or VL2-CDR3 comprising the amino acid sequence set forth in SEQ ID NO: 24. The Arginase 1 binders specifically bind arginase 1 and inhibit arginase 1 activity. CDR sequences are determined using the IMGT numbering scheme.

In particular embodiments of the invention, the Arginase 1 binder comprises (a) a VH2-CDR1 comprising the amino acid sequence set forth in SEQ ID NO: 16, a VH2-CDR2 comprising the amino acid sequence set forth in SEQ ID NO: 17, and a VH2-CDR3 comprising the amino acid sequence set forth in SEQ ID NO: 18; and (b) a VL1-CDR1 or VL2-CDR1 comprising the amino acid sequence set forth in SEQ ID NO: 25, a VL1-CDR2 or VL2-CDR2 comprising the amino acid sequence AA, and a VL1-CDR3 or VL2-CDR3 comprising the amino acid sequence set forth in SEQ ID NO: 24. The Arginase 1 binders specifically bind arginase 1 and inhibit arginase 1 activity. CDR sequences are determined using the IMGT numbering scheme.

In particular embodiments of the invention, the Arginase 1 binder comprises (a) a VH1-CDR1 or VH2-CDR1 comprising the amino acid sequence set forth in SEQ ID NO: 19, a VH1-CDR2 or VH2-CDR2 comprising the amino acid sequence set forth in SEQ ID NO: 20, and a VH1-CDR3 or VH2-CDR3 comprising the amino acid sequence set forth in SEQ ID NO: 21; and (b) a VL1-CDR1 or VL2-CDR1 comprising the amino acid sequence set forth in SEQ ID NO: 26, a VL1-CDR2 or VL2-CDR2 comprising the amino acid sequence set forth in SEQ ID NO: 27, and a VL1-CDR3 or VL2-CDR3 comprising the amino acid sequence set forth in SEQ ID NO: 28. The Arginase 1 binders specifically bind arginase 1 and inhibit arginase 1 activity. CDR sequences are determined using the Contact numbering scheme.

In further embodiments, the Arginase 1 binder comprises a VH comprising the amino acid sequence set forth for VH1 in SEQ ID NO: 2 and a VL comprising the amino acid sequence set forth in SEQ ID NO: 4.

In further embodiments, the Arginase 1 binder comprises a VH comprising the amino acid sequence set forth for VH2 in SEQ ID NO: 3 and a VL comprising the amino acid sequence set forth in SEQ ID NO: 5.

In further embodiments of the invention, the Arginase 1 binder comprises a heavy chain constant domain of the IgG1, IgG2, IgG3, or IgG4 isotype. In particular embodiments, the heavy chain constant domain comprises 1, 2, 3, 4, 5, 6, 7, 8, 9, or 10 amino acid substitutions, additions, deletions, or combinations thereof compared to the amino acid sequence of the native IgG1, IgG2, IgG3, or IgG4 isotype, wherein the Arginase 1 binder specifically binds arginase 1 and inhibits arginase 1 activity.

In further embodiments, the arginase binder inhibits arginase 1 activity by at least 50%, 60%, 70%, 80%, or 90%. In further embodiments, the arginase binder inhibits arginase 1 activity at an IC₅₀ of less than 100 nM, 50 nM, 20 nM, or 10 nM. In a further embodiment, the IC₅₀ is about 3.3+/−0.3 nM or about 5.3 +/−0.8 nM.

In further embodiments of the invention, the Arginase 1 binder comprises a heavy chain constant domain of the human IgG1 or IgG4 isotype. In particular embodiments, the heavy chain constant domain comprises 1, 2, 3, 4, 5, 6, 7, 8, 9, or 10 amino acid substitutions, additions, deletions, or combinations thereof compared to the amino acid sequence of the native IgG1 or IgG4 isotype, wherein the Arginase 1 binder specifically binds arginase 1 and inhibits arginase 1 activity. In a further aspect, the heavy chain constant domain is of the IgG4 isotype and further includes a substitution of the serine residue at position 228 (EU numbering) with proline, which corresponds to position 108 of SEQ ID NO: 44 (Serine at position 108).

In further aspects or embodiments of the invention, the antibody comprises a IgG1 heavy chain constant domain comprising the amino acid sequence shown in SEQ ID NO: 29 or a variant thereof comprising 1, 2, 3, 4, 5, 6, 7, 8, 9, or 10 amino acid substitutions, additions, deletions, or combinations thereof, wherein the Arginase 1 binder specifically binds arginase 1 and inhibits arginase 1 activity.

In further aspects or embodiments of the invention, the antibody comprises a IgG2 heavy chain constant domain comprising the amino acid sequence shown in SEQ ID NO: 37 and further comprising 1, 2, 3, 4, 5, 6, 7, 8, 9, or 10 amino acid substitutions, additions, deletions, or combinations thereof, wherein the Arginase 1 binder specifically binds arginase 1 and inhibits arginase 1 activity.

In further aspects or embodiments of the invention, the antibody comprises a IgG4 heavy chain constant domain comprising the amino acid sequence shown in SEQ ID NO: 44 and further comprising 1, 2, 3, 4, 5, 6, 7, 8, 9, or 10 amino acid substitutions, additions, deletions, or combinations thereof, wherein the Arginase 1 binder specifically binds arginase 1 and inhibits arginase 1 activity.

In particular embodiments of the invention, the constant domains as disclosed herein may comprise a C-terminal lysine or lack either a C-terminal lysine or a C-terminal glycine-lysine dipeptide.

In any one of the embodiments disclosed herein, the light chain may comprise a human kappa light chain constant domain comprising SEQ ID NO: 49 or a lambda light chain constant domain comprising SEQ ID NO: 52.

Arginase 1 binders Comprising an Effector-silent Fc Domain

Effector-silent Arginase 1 binders of the present invention are antibodies that comprise an HC constant domain or Fc domain thereof that has been modified such that the antibody displays no measurable binding to one or more FcRs or displays reduced binding to one or more FcRs compared to that of an unmodified antibody of the same IgG isotype. The effector-silent antibodies may in further embodiments display no measurable binding to each of FcγRIIIa, FcγRIIa, and FcγRI or display reduced binding to each of FcγRIIIa, FcγRIIa, and FcγRI compared to that of an unmodified antibody of the same IgG isotype. In particular embodiments, the HC constant domain or Fc domain is a human HC constant domain or Fc domain.

In particular embodiments, the effector-silent antibody comprises an Fc domain of an IgG1 or IgG2, IgG3, or IgG4 isotype that has been modified to lack N-glycosylation of the asparagine (Asn) residue at position 297 (Eu numbering system) of the HC constant domain. The consensus sequence for N-glycosylation is Asn-Xaa-Ser/Thr (wherein Xaa at position 298 is any amino acid except Pro); in all four isotypes the N-glycosylation consensus sequence is Asn-Ser-Thr. The modification may be achieved by replacing the codon encoding the Asn at position 297 in the nucleic acid molecule encoding the HC constant domain with a codon encoding another amino acid, for example Ala, Asp, Gln, Gly, or Glu, e.g. N297A, N297Q, N297G, N297E, or N297D. Alternatively, the codon for Ser at position 298 may be replaced with the codon for Pro or the codon for Thr at position 299 may be replaced with any codon except the codon for Ser. In a further alternative each of the amino acids comprising the N-glycosylation consensus sequence is replaced with another amino acid. Such modified IgG molecules have no measurable effector function. In particular embodiments, these mutated HC molecules may further comprise 1, 2, 3, 4, 5, 6, 7, 8, 9, or 10 additional amino acid substitutions, insertions, and/or deletions, wherein said substitutions may be conservative mutations or non-conservative mutations. In further embodiments, such IgGs modified to lack N-glycosylation at position 297 may further include one or more additional mutations disclosed herein for eliminating measurable effector function.

An exemplary IgG1 HC constant domain mutated at position 297, which abolishes the N-glycosylation of the HC constant domain, is set forth in SEQ ID NO: 36, an exemplary IgG2 HC constant domain mutated at position 297, which abolishes the N-glycosylation of the HC constant, is set forth in SEQ ID NO: 42, and an exemplary IgG4 HC constant domain mutated at position 297 to abolish N-glycosylation of the HC constant domain is set forth in SEQ ID NO: 48. In particular embodiments, these mutated HC molecules may further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the Fc domain of the IgG1 IgG2, IgG3, or IgG4 HC constant domain comprising the effector-silent antibody is modified to include one or more amino acid substitutions selected from E233P, L234A, L235A, L235E, N297A, N297D, D265S, and P331S (wherein the positions are identified according to Eu numbering) and wherein said HC constant domain is effector-silent. In particular embodiments, the modified IgG1 further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the HC constant domain comprises L234A, L235A, and D265S substitutions (wherein the positions are identified according to Eu numbering). In particular embodiments, the HC constant domain comprises an amino acid substitution at position Pro329 and at least one further amino acid substitution selected from E233P, L234A, L235A, L235E, N297A, N297D, D265S, and P331S (wherein the positions are identified according to Eu numbering). These and other substitutions are disclosed in WO9428027; WO2004099249; WO20121300831, U.S. Pat. Nos. 9,708,406; 8,969,526; and 9,296,815; Sondermann et al. Nature 406, 267-273 (2000)).

In particular embodiments of the above, the HC constant domain comprises an L234A/L235A/D265A; L234A/L235A/P329G; L235E; D265A; D265A/N297G; or V234A/G237A/P238S/H268A/V309L/A330S/P331S substitutions, wherein the positions are identified according to Eu numbering. In particular embodiments, the HC molecules further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the effector-silent antibody comprises an IgG1 isotype, in which the Fc domain of the HC constant domain has been modified to be effector-silent by substituting the amino acids from position 233 to position 236 of the IgG1 with the corresponding amino acids of the human IgG2 HC and substituting the amino acids at positions 327, 330, and 331 with the corresponding amino acids of the human IgG4 HC, wherein the positions are identified according to Eu numbering (Armour et al., Eur. J. Immunol. 29(8):2613-24 (1999); Shields et al., J. Biol. Chem. 276(9):6591-604(2001)). In particular embodiments, the modified IgG1 further comprise 1, 2, 3, 4, 5, 6, 7, 8, 9, or 10 additional amino acid substitutions, insertions, and/or deletions, wherein said substitutions may be conservative mutations or non-conservative mutations.

In particular embodiments, the effector-silent antibody comprises a VH fused or linked to a hybrid human immunoglobulin HC constant domain, which includes a hinge region, a CH2 domain and a CH3 domain in an N-terminal to C-terminal direction, wherein the hinge region comprises an at least partial amino acid sequence of a human IgD hinge region or a human IgG1 hinge region; and the CH2 domain is of a human IgG4 CH2 domain, a portion of which, at its N-terminal region, is replaced by 4-37 amino acid residues of an N-terminal region of a human IgG2 CH2 or human IgD CH2 domain. Such hybrid human HC constant domain is disclosed in U.S. Pat. No. 7,867,491, which is incorporated herein by reference in its entirety.

In particular embodiments, the effector-silent antibody comprises an IgG4 HC constant domain in which the serine at position 228 according to the Eu system is substituted with proline, see for example SEQ ID NO: 44. This modification prevents formation of a potential inter-chain disulfide bond between the cysteines at positions Cys226 and Cys229 in the EU numbering scheme and which may interfere with proper intra-chain disulfide bond formation. See Angal et al. Mol. Imunol. 30:105 (1993); see also (Schuurman et al., Mol. Immunol. 38: 1-8, (2001)). In further embodiments, the IgG4 constant domain includes in addition to the S228P substitution, a P239G, D265A, or D265A/N297G amino acid substitution, wherein the positions are identified according to Eu numbering. In particular embodiments of the above, the IgG4 HC constant domain is a human HC constant domain. In particular embodiments, the HC molecules further comprise 1, 2, 3, 4, 5, 6, 7, 8, 9, or 10 additional amino acid substitutions, insertions, and/or deletions, wherein said substitutions may be conservative mutations or non-conservative mutations.

Exemplary IgG1 HC constant domains include HC constant domains comprising an amino acid sequence selected from the group consisting of amino acid sequences set forth in SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 50, and SEQ ID NO: 51. Exemplary IgG2 HC constant domains have an amino acid sequence selected from the group consisting of amino acid sequences set forth in SEQ ID NO: 38, SEQ ID NO: 39, SEQ ID NO: 40, SEQ ID NO: 41, SEQ ID NO: 42, and SEQ ID NO: 43. Exemplary IgG4 HC constant domains have an amino acid sequence selected from the group consisting of amino acid sequences set forth in SEQ ID NO: 44, SEQ ID NO: 45, SEQ ID NO: 46, SEQ ID NO: 47, and SEQ ID NO: 48.

In particular embodiments of the Arginase 1 binder , the Arginase 1 binder is an antibody comprising an IgG1, IgG2, or IgG4 Fc domain as disclosed herein, which further comprises a C-terminal lysine or lack either a C-terminal lysine or a C-terminal glycine-lysine dipeptide.

In any one of the embodiments disclosed herein, the light chain may comprise a human kappa light chain constant domain comprising SEQ ID NO: 49 or a lambda light chain constant domain comprising SEQ ID NO: 52.

### Nucleic Acid Molecules Encoding the Arginase 1 Binders

The present invention further provides nucleic acid molecules that encode the Arginase 1 binders of the present invention. In particular embodiments, the Arginase 1 binder comprises a VH1 or VH2 domain encoded by a nucleic acid molecule comprising the nucleotide sequence set forth in SEQ ID NO: 55 or SEQ ID NO: 56, respectively. In a further embodiment, Arginase 1 binder further comprises a VL1 or VL2 domain encoded by a nucleic acid molecule comprising the nucleotide sequence set forth in SEQ ID NO: 57 or SEQ ID NO: 58, respectively.

In further embodiments, the Arginase 1 binder comprises a VH1 or VH2 domain encoded by a nucleic acid molecule comprising the nucleotide sequence set forth in SEQ ID NO: 55 or SEQ ID NO: 56, respectively, and a VL1 or VL2 domain encoded by a nucleic acid molecule comprising the nucleotide sequence set forth in SEQ ID NO: 57 or SEQ ID NO: 58, respectively.

In further embodiments, the Arginase 1 binder comprises a VH1 or VH2 domain encoded by a nucleic acid molecule comprising the nucleotide sequence set forth in SEQ ID NO: 55 or SEQ ID NO: 56, respectively, and a VL1 or VL2 domain encoded by a nucleic acid molecule comprising the nucleotide sequence set forth in SEQ ID NO: 57 or SEQ ID NO: 58, respectively, wherein the nucleic acid molecule encoding the VH1 or VH2 is linked to a nucleic acid molecule encoding an IgG1, IgG2, IGG3, or IgG4 heavy chain constant domain and the nucleic acid molecule encoding the VL is linked to a nucleic acid molecule encoding kappa or lambda light chain constant domain.

The present invention further provides nucleic acid molecules that encode the Arginase 1 binders of the present invention. In particular embodiments, the Arginase 1 binder comprises a VH2domain encoded by a nucleic acid molecule comprising the nucleotide sequence set forth in SEQ ID NO: 56. In a further embodiment, Arginase 1 binder further comprises a VL2 domain encoded by a nucleic acid molecule comprising the nucleotide sequence set forth in SEQ ID NO: 58.

In further embodiments, the Arginase 1 binder comprises a VH2 domain encoded by a nucleic acid molecule comprising the nucleotide sequence set forth in SEQ ID NO: 56 and a VL2 domain encoded by a nucleic acid molecule comprising the nucleotide sequence set forth in SEQ ID NO: 58.

In further embodiments, the Arginase 1 binder comprises a VH2 domain encoded by a nucleic acid molecule comprising the nucleotide sequence set forth in SEQ ID NO: 56 and a VL2 domain encoded by a nucleic acid molecule comprising the nucleotide sequence set forth in SEQ ID NO: 58, wherein the nucleic acid molecule encoding the VH1 or VH2 is linked to a nucleic acid molecule encoding an IgG1, IgG2, IGG3, or IgG4 heavy chain constant domain and the nucleic acid molecule encoding the VL is linked to a nucleic acid molecule encoding kappa or lambda light chain constant domain.

Exemplary IgG1 heavy chain constant domains may be encoded by a nucleic acid molecule comprising the nucleotide sequence set forth in SEQ ID NO: 59, SEQ ID NO: 60, SEQ ID NO: 61, SEQ ID NO: 62, SEQ ID NO: 63, SEQ ID NO: 64, SEQ ID NO: 65, SEQ ID NO: 66, SEQ ID NO: 80, or SEQ ID NO: 81.

Exemplary IgG2 heavy chain constant domains may be encoded by a nucleic acid molecule comprising the nucleotide sequence set forth in SEQ ID NO: 67, SEQ ID NO: 68, SEQ ID NO: 69, SEQ ID NO: 70, SEQ ID NO: 71, SEQ ID NO: 72, or SEQ ID NO: 73.

Exemplary IgG4 heavy chain constant domains may be encoded by a nucleic acid molecule comprising the nucleotide sequence set forth in SEQ ID NO: 74, SEQ ID NO: 75, SEQ ID NO: 76, SEQ ID NO: 77, or SEQ ID NO: 78.

Exemplary light chain constant domains may be encoded by a nucleic acid molecule comprising the nucleotide sequence set forth in SEQ ID NO: 79 or SEQ ID NO: 83.

In particular embodiments, the HC and LC (or VH and VL) are expressed as a fusion protein in which the N-terminus of the HC and the LC (or VH and VL) are fused to a leader peptide to facilitate the transport of the antibody through the secretory pathway. Examples of leader peptides that may be used include MSVPTQVLGLLLLWLTDARC (SEQ ID NO: 84) encoded by the nucleotide sequence set forth in SEQ ID NO: 86 or MEWSWVFLFFLSVTTGVHS (SEQ ID NO: 85) encoded by the nucleotide sequence set forth in SEQ ID NO: 87. Thus, in particular embodiments, the aforementioned nucleic acid molecules may comprise a polynucleotide encoding a leader peptide linked to the 5′ end of the nucleic acid molecule.

The nucleic acid molecules disclosed herein may include one or more substitutions that optimize one or more of the codons for enhancing the expression of the nucleic acid molecule in a particular host cell, e.g., yeast or fungal host cell, non-human mammalian hot cell, human host cell, insect host cell, or prokaryote host cell. Methods and computer programs for optimizing a nucleic acid molecule for enhancing expression in a particular host cell are well known in the art, e.g. the IDT Codon Optimization Tool commercially available from Integrated DNA Technologies, Inc. 1710 Commercial Park, Coralville, Iowa 52241, USA.; U.S. Pat. No. 8,326,547; WO2020024917A1.

Methods for making Arginase 1 binders

The present invention includes recombinant methods for making Arginase 1 binders comprising introducing into a host cell (i) an expression vector that encodes the VH and VL of an Arginase 1 binder or the HC and LC of an Arginase 1 binder, or (ii) two expression 35 vectors, one encoding the VH of an Arginase 1 binder or the HC of an Arginase 1 binder the other encoding the VL of an Arginase 1 binder or the LC of an Arginase 1 binder. The nucleic acid molecules or polynucleotides encoding the VH, VL, HC, or LC are operably linked to a promoter and other transcription and translation regulatory sequences (as used herein “VH” refers to either VH1or VH2and the term “VL” refers to both VL1and VL2). The host cell is cultured under conditions and a time period suitable for expression of the nucleic acid molecules followed by isolating the Arginase 1 binder from the host cell and/or medium in which the host cell is grown. See e.g., WO 04/041862, WO 2006/122786, WO 2008/020079, WO 2008/142164 or WO 2009/068627. The expression vector may be a plasmid or viral vector. The invention also relates to hosts or host cells that contain such nucleic acid molecule encoding the Arginase 1 binders or components thereof, e.g., solely the VH or HC or solely the VL or HC.

Eukaryotic and prokaryotic host cells, including mammalian cells as hosts for expression of the Arginase 1 binder are well known in the art and include many immortalized cell lines available from the American Type Culture Collection (ATCC). These include, but are not limited to, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, HEK-293 cells and a number of other cell lines. Thus, mammalian host cells include human, mouse, rat, dog, monkey, pig, goat, bovine, horse and hamster cells. Cell lines of particular preference are selected through determining which cell lines have high expression levels. Other cell lines that may be used are insect cell lines (e.g., Spodoptera frugiperda or Trichoplusia ni), amphibian cells, bacterial cells, plant cells and fungal cells. Fungal cells include yeast and filamentous fungus cells including, for example, Pichia pastoris, Saccharomyces cervisiea, and Trichoderma reesei. The present invention includes any host cell comprising an Arginase 1 binder of the present invention or comprising one or more nucleic acid molecules encoding such an Arginase 1 binder or comprising an expression vector that comprises one or more nucleic acid molecules encoding such Arginase 1 binder.

Further, expression of an Arginase 1 binder from production cell lines can be enhanced using a number of known techniques. For example, the glutamine synthetase gene expression system (the GS system) is a common approach for enhancing expression under certain conditions. The GS system is discussed in whole or part in connection with European Patent Nos. 0 216 846, 0 256 055, and 0 323 997 and European Patent Application No. 89303964.4. Thus, in an embodiment of the invention, the mammalian host cells lack a glutamine synthetase gene and are grown in the absence of glutamine in the medium wherein, however, the nucleic acid molecule encoding the immunoglobulin chain comprises a glutamine synthetase gene which complements the lack of the gene in the host cell. Such host cells containing the Arginase lbinder or nucleic acid(s) or expression vector(s) as discussed herein as well as expression methods, as discussed herein, for making the Arginase 1 binder using such a host cell are part of the present invention.

The present invention includes methods for purifying an Arginase 1 binder comprising introducing a sample (e.g., culture medium, cell lysate or cell lysate fraction, e.g., a soluble fraction of the lysate) comprising the Arginase 1 binder to a purification medium (e.g., cation-exchange medium, anion-exchange medium and/or hydrophobic exchange medium) and either collecting purified Arginase 1 binder from the flow-through fraction of said sample that does not bind to the medium; or, discarding the flow-through fraction and eluting bound Arginase 1 binder from the medium and collecting the eluate. In an embodiment of the invention, the medium is in a column to which the sample is applied. In an embodiment of the invention, the purification method is conducted following recombinant expression of the Arginase 1 binder in a host cell, e.g., wherein the host cell is first lysed and, optionally, the lysate is purified of insoluble materials prior to purification on a medium; or wherein the Arginase 1 binder is secreted into the culture medium by the host cell and the medium or a fraction thereof is applied to the purification medium.

In general, glycoproteins produced in a particular cell line or transgenic animal will have a glycosylation pattern that is characteristic for glycoproteins produced in the cell line or transgenic animal. Therefore, the particular glycosylation pattern of an Arginase 1 binder will depend on the particular cell line or transgenic animal used to produce the Arginase 1 binder. Arginase 1 binders comprising only non-fucosylated N-glycans are part of the present invention and may be advantageous, because non-fucosylated antibodies have been shown to typically exhibit more potent efficacy than their fucosylated counterparts both in vitro and in vivo (See for example, Shinkawa et al., J. Biol. Chem. 278: 3466-3473 (2003); U.S. Patent Nos. 6,946,292 and 7,214,775). These Arginase 1 binders with non-fucosylated N-glycans are not likely to be immunogenic because their carbohydrate structures are a normal component of the population that exists in human serum IgG.

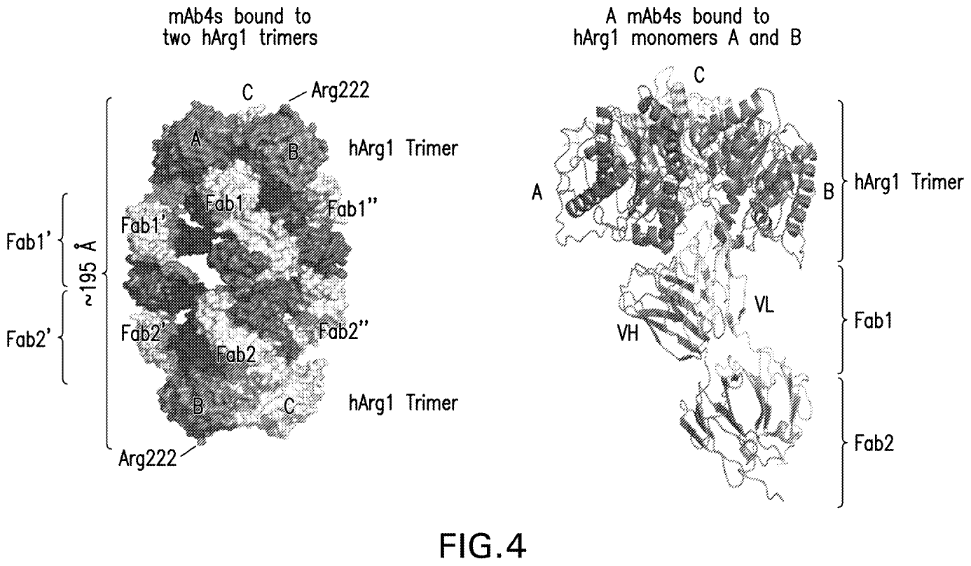

The present invention includes Arginase 1 binders comprising N-linked glycans that are typically added to immunoglobulins produced in Chinese hamster ovary cells (CHO N-linked glycans) or to engineered yeast cells (engineered yeast N-linked glycans), such as, for example, Pichia pastoris. For example, in an embodiment of the invention, the Arginase 1 binder comprises one or more of the “engineered yeast N-linked glycans” or “CHO N-linked glycans” (e.g., G0 and/or G0-F and/or G1 and/or G1-F and/or and/or G2-F and/or Man5, see FIG. 4). In an embodiment of the invention, the Arginase lbinder comprises the engineered yeast N-linked glycans, i.e., G0 and/or G1 and/or G2, optionally, further including Man5. In an embodiment of the invention, the Arginase 1 binders comprise the CHO N-linked glycans, i.e., G0-F, G1-F and G2-F, optionally, further including G0 and/or G1 and/or G2 and/or Man5. In an embodiment of the invention, about 80% to about 95% (e.g., about 80-90%, about 85%, about 90% or about 95%) of all N-linked glycans on the Arginase 1 binders are engineered yeast N-linked glycans or CHO N-linked glycans. See Nett et al. Yeast. 28(3): 237-252 (2011); Hamilton et al. Science. 313(5792): 1441-1443 (2006); Hamilton et al. Curr Opin Biotechnol. 18(5): 387-392 (2007). For example, in an embodiment of the invention, an engineered yeast cell is GFI5.0 or YGLY8316 or strains set forth in U.S. Patent No. 7,795,002 or Zha et al. Methods Mol Biol. 988:31-43 (2013). See also international patent application publication no. WO2013/066765.

### Administration/Pharmaceutical Compositions

The Arginase 1 binder may be provided in suitable pharmaceutical compositions comprising the Arginase 1 binder and a pharmaceutically acceptable carrier. The carrier may be diluent, adjuvant, excipient, or vehicle with which the Arginase 1 binder is administered. Such vehicles may be liquids, such as water and oils, including those of petroleum, animal, vegetable or synthetic origin, such as peanut oil, soybean oil, mineral oil, sesame oil and the like. For example, 0.4% saline and 0.3% glycine may be used. These solutions are sterile and generally free of particulate matter. They may be sterilized by conventional, well-known sterilization techniques (e.g., filtration). The compositions may contain pharmaceutically acceptable auxiliary substances as required to approximate physiological conditions such as pH adjusting and buffering agents, stabilizing, thickening, lubricating and coloring agents, etc. The concentration of the molecules or of the invention in such pharmaceutical formulation may vary widely, i.e., from less than about 0.5%, usually to at least about 1% to as much as 15 or 20% by weight and will be selected primarily based on required dose, fluid volumes, viscosities, etc., according to the particular mode of administration selected. Suitable vehicles and formulations, inclusive of other human proteins, e.g., human serum albumin, are described, for example, in e.g. Remington: The Science and Practice of Pharmacy, 21.sup.st Edition, Troy, D. B. ed., Lipincott Williams and Wilkins, Philadelphia, Pa. 2006, Part 5, Pharmaceutical Manufacturing pp 691-1092, see especially pp. 958-989.

The mode of administration of the Arginase 1 binder may be any suitable route such as parenteral administration, e.g., intradermal, intramuscular, intraperitoneal, intravenous or subcutaneous, pulmonary, transmucosal (oral, intranasal, intravaginal, rectal) or other means appreciated by the skilled artisan, as well known in the art.

The Arginase 1 binder may be administered to an individual (e.g., patient) by any suitable route, for example parentally by intravenous (i.v.) infusion or bolus injection, intramuscularly or subcutaneously, or intraperitoneally. i.v. infusion may be given over for, example, 15, 30, 60, 90, 120, 180, or 240 minutes, or from 1, 2, 3, 4, 5, 6, 7, 8, 9, 10, 11 or 12 hours.

The dose given to an individual having cancer or malignancy is sufficient to alleviate or at least partially arrest the disease being treated (“therapeutically effective amount”) and may be sometimes 0.005 mg/kg to about 100 mg/kg, e.g. about 0.05 mg/kg to about 30 mg/kg or about 5 mg to about 25 mg/kg, or about 4 mg/kg, about 8 mg/kg, about 16 mg/kg or about 24 mg/kg, or, e.g., about 1, 2, 3, 4, 5, 6, 7, 8, 9 or 10 mg/kg, but may even higher, for example about 15, 16, 17, 18, 19, 20, 21, 22, 23, 24, 25, 30, 40, 50, 60, 70, 80, 90 or 100 mg/kg.

A fixed unit dose may also be given, for example, 50, 100, 200, 500 or 1000 mg, or the dose may be based on the patient's surface area, e.g., 500, 400, 300, 250, 200, or 100 mg/m². Usually between 1 and 8 doses, (e.g., 1, 2, 3, 4, 5, 6, 7 or 8) may be administered to treat cancer or malignancy, but 9, 10, 11, 12, 13, 14, 15, 16, 17, 18, 19, 20 or more doses may be given.

The administration of the Arginase 1 binder may be repeated after one day, two days, three days, four days, five days, six days, one week, two weeks, three weeks, one month, five weeks, six weeks, seven weeks, two months, three months, four months, five months, six months or longer. Repeated courses of treatment are also possible, as is chronic administration. The repeated administration may be at the same dose or at a different dose. For example, the Arginase 1 binder in the methods of the invention may be administered at 8 mg/kg or at 16 mg/kg at weekly interval for 8 weeks, followed by administration at 8 mg/kg or at 16 mg/kg every two weeks for an additional 16 weeks, followed by administration at 8 mg/kg or at 16 mg/kg every four weeks by intravenous infusion.

The Arginase 1 binder may be administered by maintenance therapy, such as, e.g., once a week for a period of 6 months or more. For example, Arginase 1 binder in the methods of the invention may be provided as a daily dosage in an amount of about 0.1-100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 31, 32, 33, 34, 35, 36, 37, 38, 39, or 40, or alternatively, at least one of week 1, 2, 3, 4, 5, 6, 7, 8, 9, 10, 11, 12, 13, 14, 15, 16, 17, 18, 19 or 20 after initiation of treatment, or any combination thereof, using single or divided doses of every 24, 12, 8, 6, 4, or 2 hours, or any combination thereof.

The Arginase 1 binder may also be administered prophylactically in order to reduce the risk of developing cancer, delay the onset of the occurrence of an event in cancer progression, and/or reduce the risk of recurrence when a cancer is in remission. This may be especially useful in patients wherein it is difficult to locate a tumor that is known to be present due to other biological factors.

The Arginase 1 binder may be lyophilized for storage and reconstituted in a suitable carrier prior to use. This technique has been shown to be effective with conventional protein preparations and well known lyophilization and reconstitution techniques can be employed.

### Combination Therapy Treatments

The combination therapy of the present invention comprises an Arginase 1 binder and another therapeutic agent (small molecule or antibody) may be used for the treatment any proliferative disease, in particular, treatment of cancer. In particular embodiments, the combination therapy of the present invention may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy of the present invention may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

### Combination Therapy Comprising an Arginase 1 Binder and a Chemotherapy Agent

The combination therapy of the present invention may be administered to an individual having a cancer in combination with chemotherapy. The individual may undergo the chemotherapy at the same time the individual is undergoing the combination therapy of the present invention. The individual may undergo the combination therapy of the present invention after the individual has completed chemotherapy. The individual may be administered the chemotherapy after completion of the combination therapy. The combination therapy of the present invention may also be administered to an individual having recurrent or metastatic cancer with disease progression or relapse cancer and who is undergoing chemotherapy or who has completed chemotherapy.

The chemotherapy may include a chemotherapy agent selected from the group consisting of
(i) alkylating agents, including but not limited to, bifunctional alkylators, cyclophosphamide, mechlorethamine, chlorambucil, and melphalan;
(ii) monofunctional alkylators, including but not limited to, dacarbazine, nitrosoureas, and temozolomide (oral dacarbazine);
(iii) anthracyclines, including but not limited to, daunorubicin, doxorubicin, epirubicin, idarubicin, mitoxantrone, and valrubicin;
(iv) cytoskeletal disruptors (taxanes), including but not limited to, paclitaxel, docetaxel, abraxane, and taxotere;
(v) epothilones, including but not limited to, ixabepilone, and utidelone;
(vi) histone deacetylase inhibitors, including but not limited to, vorinostat, and romidepsin;
(vii) inhibitors of topoisomerase i, including but not limited to, irinotecan, and topotecan;
(viii) inhibitors of topoisomerase ii, including but not limited to, etoposide, teniposide, and tafluposide;
(ix) kinase inhibitors, including but not limited to, bortezomib, erlotinib, gefitinib, imatinib, vemurafenib, and vismodegib;
(x) nucleotide analogs and precursor analogs, including but not limited to, azacitidine, azathioprine, fluoropyrimidines (e.g., such as capecitabine, carmofur, doxifluridine, fluorouracil, and tegafur) cytarabine, gemcitabine, hydroxyurea, mercaptopurine, methotrexate, and tioguanine (formerly thioguanine);
(xi) peptide antibiotics, including but not limited to, bleomycin and actinomycin; a platinum-based agent, including but not limited to, carboplatin, cisplatin, and oxaliplatin;
(xii) retinoids, including but not limited to, tretinoin, alitretinoin, and bexarotene; and
(xiii) vinca alkaloids and derivatives, including but not limited to, vinblastine, vincristine, vindesine, and vinorelbine.

Selecting a dose of the chemotherapy agent for chemotherapy depends on several factors, including the serum or tissue turnover rate of the entity, the level of symptoms, the immunogenicity of the entity, and the accessibility of the target cells, tissue or organ in the individual being treated. The dose of the additional therapeutic agent should be an amount that provides an acceptable level of side effects. Accordingly, the dose amount and dosing frequency of each additional therapeutic agent will depend in part on the particular therapeutic agent, the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, NY; Bach (ed.) (1993) Monoclonal Antibodies and Peptide Therapy in Autoimmune Diseases, Marcel Dekker, New York, NY; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dose regimen may be made by the clinician, e.g., using parameters or factors known or suspected in the art to affect treatment or predicted to affect treatment, and will depend, for example, the individual's clinical history (e.g., previous therapy), the type and stage of the cancer to be treated and biomarkers of response to one or more of the therapeutic agents in the combination therapy.

Thus, the present invention contemplates embodiments of the combination therapy of the present invention that further includes a chemotherapy step comprising platinum-containing chemotherapy, pemetrexed and platinum chemotherapy or carboplatin and either paclitaxel or nab-paclitaxel. In particular embodiments, the combination therapy with a chemotherapy step may be used for treating at least NSCLC and HNSCC.

The combination therapy further in combination with a chemotherapy step may be used for the treatment any proliferative disease, in particular, treatment of cancer. In particular embodiments, the combination therapy of the present invention may be used to treat melanoma, non-small cell lung cancer, head and neck cancer, urothelial cancer, breast cancer, gastrointestinal cancer, multiple myeloma, hepatocellular cancer, non-Hodgkin lymphoma, renal cancer, Hodgkin lymphoma, mesothelioma, ovarian cancer, small cell lung cancer, esophageal cancer, anal cancer, biliary tract cancer, colorectal cancer, cervical cancer, thyroid cancer, or salivary cancer.

In another embodiment, the combination therapy further in combination with a chemotherapy step may be used to treat pancreatic cancer, bronchus cancer, prostate cancer, pancreatic cancer, stomach cancer, ovarian cancer, urinary bladder cancer, brain or central nervous system cancer, peripheral nervous system cancer, uterine or endometrial cancer, cancer of the oral cavity or pharynx, liver cancer, kidney cancer, testicular cancer, biliary tract cancer, small bowel or appendix cancer, adrenal gland cancer, osteosarcoma, chondrosarcoma, or cancer of hematological tissues.

In particular embodiments, the combination therapy with a chemotherapy step may be used to treat one or more cancers selected from melanoma (metastatic or unresectable), primary mediastinal large B-cell lymphoma (PMBCL), urothelial carcinoma, MSIHC, gastric cancer, cervical cancer, hepatocellular carcinoma (HCC), Merkel cell carcinoma (MCC), renal cell carcinoma (including advanced), and cutaneous squamous carcinoma.

### Combination Therapy Comprising an Arginase 1 Binder and a Therapeutic Antibody

The Arginase 1 binder of the present invention may be administered in combination with one or more therapeutic agent, which is an antibody, for treatment of cancer or proliferative disease. The individual may undergo treatment with the therapeutic antibody at the same time the individual is undergoing the combination therapy of the present invention. The individual may undergo the combination therapy of the present invention after the individual has completed treatment with the therapeutic antibody. The individual may be administered the treatment with the therapeutic antibody after completion of the combination therapy. The combination therapy of the present invention may also be administered to an individual having recurrent or metastatic cancer with disease progression or relapse cancer and who is undergoing chemotherapy or who has completed chemotherapy. In particular embodiments, the therapeutic agent targets the programmed death 1 receptor or ligand., PD-1 and PD-L1, respectively.

Exemplary anti-PD-1 antibodies that may be used in a combination therapy with the Arginase 1 binder include any antibody that binds PD-1 and inhibits PD-1 from binding PD-L1. In a further embodiment, the exemplary anti-PD-1 antibody is selected from the group consisting of nivolumab, pembrolizumab, and cemiplimab-rwlc. Exemplary antibodies include the following anti-PD-1 antibodies and compositions comprising an anti-PD1 antibody and a pharmaceutically acceptable salt.

Pembrolizumab, also known as KEYTRUDA, lambrolizumab, MK-3475 or SCH-900475, is a humanized anti-PD-1 antibody described in U.S. Pat. No. 8,354,509 and WO2009/114335 and disclosed, e.g., in Hamid, et al., New England J. Med. 369 (2): 134-144 (2013).

Nivolumab, also known as OPDIVO, MDX-1106-04, ONO-4538, or BMS-936558, is a fully human IgG4 anti-PD-1 antibody described in WO2006/121168 and U.S. Pat. No. 8,008,449.

Cemiplimab-rwlc, also known as cemiplimab, LIBTAYO or REGN2810, is a recombinant human IgG4 monoclonal antibody that is described in WO2015112800 and U.S. Pat. No. 9,987,500.

In particular embodiments, the anti-PD-1 antibody comprises (i) a VH comprising the three HC-CDRs of pembrolizumab fused or linked to an effector-silent HC constant domain and (ii) a VL comprising the three LC-CDRs of pembrolizumab fused or linked to a LC kappa or lambda constant domain.

In particular embodiments, the anti-PD-1 antibody comprises (i) a VH comprising the three HC-CDRs of nivolumab fused or linked to an effector-silent HC constant domain and (ii) a VL comprising the three LC-CDRs of nivolumab fused or linked to a LC kappa or lambda constant domain.

In particular embodiments, the anti-PD-1 antibody comprises (i) a VH comprising the three HC-CDRs of cemiplimab-rwlc fused or linked to an effector-silent HC constant domain and (ii) a VL comprising the three LC-CDRs of nivolumab fused or linked to a LC kappa or lambda constant domain.

In particular embodiments, the anti-PD-1 antibody VH may be fused or linked to an IgG1, IgG2, IgG3, or IgG4 HC constant domain that is not currently linked to the particular VH or is linked to an IgG1, IgG2, IgG3, or IgG4 HC constant domain has been modified to include one or more mutations in the Fc domain that render the resulting anti-PD-1 antibody effecter-silent.

### Injection Device for Administering an Arginase 1 Binder

The present invention also provides an injection device comprising an Arginase 1 binder as set forth herein or a pharmaceutical composition thereof An injection device is a device that introduces a substance into the body of a patient via a parenteral route, e.g., intramuscular, subcutaneous or intravenous. For example, an injection device may be a syringe (e.g., pre-filled with the pharmaceutical composition, such as an auto-injector) which, for example, includes a cylinder or barrel for holding fluid to be injected (e.g., comprising the Arginase 1 binder or a pharmaceutical composition thereof), a needle for piecing skin and/or blood vessels for injection of the fluid; and a plunger for pushing the fluid out of the cylinder and through the needle bore. In an embodiment of the invention, an injection device that comprises an Arginase 1 binder or a pharmaceutical composition thereof is an intravenous (IV) injection device. Such a device includes the Arginase 1 binder or a pharmaceutical composition thereof in a cannula or trocar/needle which may be attached to a tube which may be attached to a bag or reservoir for holding fluid (e.g., saline; or lactated ringer solution comprising NaCl, sodium lactate, KCl, CaCl₂ and optionally including glucose) introduced into the body of the subject through the cannula or trocar/needle.

The Arginase 1 binder or a pharmaceutical composition thereof may, in an embodiment of the invention, be introduced into the device once the trocar and cannula are inserted into the vein of a subject and the trocar is removed from the inserted cannula. The IV device may, for example, be inserted into a peripheral vein (e.g., in the hand or arm); the superior vena cava or inferior vena cava, or within the right atrium of the heart (e.g., a central IV); or into a subclavian, internal jugular, or a femoral vein and, for example, advanced toward the heart until it reaches the superior vena cava or right atrium (e.g., a central venous line). In an embodiment of the invention, an injection device is an autoinjector; a jet injector or an external infusion pump. A jet injector uses a high-pressure narrow jet of liquid which penetrate the epidermis to introduce the Arginase 1 binder or a pharmaceutical composition thereof to a patient's body. External infusion pumps are medical devices that deliver the Arginase 1 binder or a pharmaceutical composition thereof into a patient's body in controlled amounts. External infusion pumps may be powered electrically or mechanically. Different pumps operate in different ways, for example, a syringe pump holds fluid in the reservoir of a syringe, and a moveable piston controls fluid delivery, an elastomeric pump holds fluid in a stretchable balloon reservoir, and pressure from the elastic walls of the balloon drives fluid delivery. In a peristaltic pump, a set of rollers pinches down on a length of flexible tubing, pushing fluid forward. In a multi-channel pump, fluids can be delivered from multiple reservoirs at multiple rates.

### Kits Comprising an Arginase 1 Binder

Further provided are kits comprising one or more components that include, but are not limited to, an Arginase 1 binder, as discussed herein in association with one or more additional components including, but not limited to, a further therapeutic agent, as discussed herein. The Arginase 1 binder and/or the therapeutic agent can be formulated as a pure composition or in combination with a pharmaceutically acceptable carrier, in a pharmaceutical composition.

In one embodiment, the kit includes an Arginase 1 binder or a pharmaceutical composition thereof in one container (e.g., in a sterile glass or plastic vial) and a further therapeutic agent in another container (e.g., in a sterile glass or plastic vial).

In another embodiment, the kit comprises a combination of the invention, including an Arginase 1 binder or pharmaceutical composition thereof in combination with one or more therapeutic agents formulated together, optionally, in a pharmaceutical composition, in a single, common container.

If the kit includes a pharmaceutical composition for parenteral administration to a subject, the kit can include a device for performing such administration. For example, the kit can include one or more hypodermic needles or other injection devices as discussed above. Thus, the present invention includes a kit comprising an injection device and the Arginase 1 binder, e.g., wherein the injection device includes Arginase 1 binder or wherein the Arginase 1 binder is in a separate vessel.

The kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the invention may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and patent information.

The following examples are intended to promote a further understanding of the present invention.

### Example 1

Expression and Purification of hArg1

Full-length untagged hArg1 was expressed in E.coli BL21 (DE3) cells using superbroth media. Expression was induced with 1 mM Isopropyl β-d-1-thiogalactopyranoside (IPTG) at OD₆₀₀ of 0.8 and cells were grown for four hours at 37° C. Cell pellets were resuspended in lysis buffer (10 mM Tris pH 7.5, 5 mM MnCl₂, 2 mM beta-mercaptoethanol (BME), 1 mg/mL lysozyme), passed through a microfluidizer three times at 15,000 pounds per square inch (PSI) and the soluble fraction was clarified by centrifugation at 11,000 xg. Clarified lysates were heat treated at 60° C. for 20 minutes. Heat treated lysates were passed through a HiTRAP-SP column (GE). Flow through containing hArg1 was diluted to about 40 mM NaCl and reloaded on another HiTRAP-SP column. hArg1 was eluted from the column using a linear gradient from 20 mM NaCl to 1 M NaCl. Pooled fractions were concentrated and loaded on a HiLoad Superdex 200 26/60 size exclusion column in 25 mM HEPES pH 7.3, 150 mM NaCl, 1 mM MnCl. Peak fractions were analyzed by SDS-PAGE, pooled and concentrated. Purification adapted from Strickland Acta Cryst. (2011). F67, 90-93.

### Example 2

### Antibody Discovery and Optimization

De novo antibody discovery for identifying mAb4 was from a hybridoma obtained from mice challenged with hArg1. Antibody mAb4 is a mouse antibody in which the VH and VL were humanized and constructed on a human IgG4 S228P/kappa backbone to produce humanized mAb4 (See FIGS. 1A-1D). The humanized mAb4 was used for the following examples unless indicated otherwise. All proteins were analytically and verified by size-exclusion chromatography (SEC) and sodium dodecyl sulfate polyacrylamide gel electrophoresis (SDS-PAGE).

### Example 3

### Antibody Production

ExpiCHO-S cells growing in suspension were transfected with indicated antibody expression plasmids (HC+LC) using commercially available protocols and ExpiFectamine CHO reagents (Thermo-Fisher)(Sivasubramanian et al., op. cit.). In brief, cells were transfected day 0 using 1 μg total DNA (3:2 ratio LC:HC) per 1 mL cells at a density of 6 million cells per mL and a viability>95% measured using a Vi-Cell (Beckman-Coulter). On day one, ExpiCHO feed and enhancer were added and culture temperature was lowered to 32° C. On day five, a second EXPI-CHO feed was performed and cell viability was measured using a Vi-Cell (Beckman-Coulter). Cultures were harvested between day 8 and day 12 depending on a cell viability greater than 80%. Antibody was purified from clarified supernatant using Protein A chromatography (mAbSelect Sure LX, GE Healthcare). Protein A was incubated with the clarified supernatant overnight in 4° C. on a roller mixer. Resin was then collected from supernatant mixture and transferred into a column and washed with 10 column volumes (CV) of PBS. Elution of antibody was achieved using 20 mM sodium acetate, pH 3.5. One column volume (CV fractions were collected and tested by Bradford assay to determine presence of protein. In some cases, Protein A purification was followed by anion exchange chromatography (Capto Q, GE Healthcare). Purified antibody was checked for purity by reduced and non-reduced Capillary electrophoresis sodium dodecyl sulfate (CE-SDS) (Perkin-Elmer), concentration was measured by A280, and aggregate content was analyzed by size exclusion ultra performance liquid chromatography (SEC-UPLC) using a BEH200 UPLC-SEC analytical column (Waters corporation). Endotoxin was quantified using Endosafe® nexgen-MCS™ (Charles River). Intact mass was confirmed via Synapt G2S QTOF or Xevo-TOF (Waters).

### Example 4

Antibody Potency and Mechanism of hArg1 Inhibition

To evaluate antibody potency and mechanism of hArg1 inhibition, the antibodies to be tested were diluted in assay buffer (50 mM Tris pH 7.5, 50 mM sodium chloride, 1 mM MnCl, and 0.05% bovine serum albumin) to a concentration 2.5-fold higher than desired assay concentrations. To each well of a Greiner black 384-well assay plate (catalog #781086) was added 10 μL of antibody solution followed by 10 μL of assay buffer or assay buffer with 1 nM human or mouse Argl. After 30 minutes of incubation at room temperature, 5 μL of a 5× solution of thioarginine (variable concentrations) was added. The assay was allowed to proceed for 60 minutes then quenched by addition of 15 μL of a solution of 375 μM 7-Diethylamine-3-(4-maleimidophenyl)-4-methylcoumarin (Sigma Chemical) in 70% ethanol to quench the reaction and detect thioornithine. The plate was briefly shaken to mix and the fluorescence was measured in a Spectramax plate reader (Molecular Devices) with a 410 nm excitation wavelength and an 490 nm emission wavelength. Kinetic data were fit to various models of enzyme inhibition (competitive, mixed, noncompetitive, and uncompetitive) using GraphPad Prism.

An alternate assessment of antibody potency was performed by serially diluting antibodies in assay buffer and performing the assay described above except that a fixed concentration of 1 mM thioarginine was used. Data were normalized to wells containing either no antibody (no effect control) or no arginine (max effect control). The percent inhibition was then fit to a four-parameter logistic equation in GraphPad Prism to determine the IC50.

### Example 5

Determination of hArg1 Activity by Liquid Chromatography-Mass Spectroscopy (LC-MS)

Arginase enzyme was diluted to 1.25 nM in assay buffer containing 50 mM Tris pH 7.5, 50 mM NaCl, 1 mM MnCl₂ and 0.05% Bovine serum albumin and 20 μL of the dilution were added to black 384 well assay plates (Greiner Bio-One Inc., Monroe, CA). Titrations of antibodies were added to appropriate wells on the plate, following which samples were incubated at 37° C. for 30 minutes in an oven. The arginine substrate was diluted in assay buffer to 1.5 mM and 5 μL were added into assay plate (final substrate concentration=300 μM). The reaction was carried out for one hour at 37° C. At the end of the reaction, 15 μL of ethanol containing internal standards (40 μM [¹³C6]-Arginine and [¹³C5]-Ornithine) were added to the mixture to quench the reaction. The assay plate was then stored at −80° C. until sample derivatization.

Then, 10 μL of thawed sample were transferred from the assay plate to a 384 deep-well plate and 90 μL methanol were added. Samples were centrifuged at 2,500 rpm for 10 minutes at room temperature and five μL of supernatant were transferred to a fresh 384 well SiliGuard-coated plate containing 35 μL of neat borate buffer. AccQTag Ultra reagent (Waters Corporation, Milford, MA) was initially prepared according to the manufacturer's instructions and then diluted an additional two-fold with acetonitrile. Then, 10 mL of the reconstituted AccQTag were added to each well and plates were sealed with pierceable aluminum foil (Agilent Technologies, Santa Clara, CA). The derivatization reaction was carried out by incubating at 55° C. in an oven for 30 minutes. Plates were then stored at 4° C. prior to LC-MS analysis.

Samples were analyzed on a Thermo TSQ Vantage triple quadrupole mass spectrometer with an electrospray ionization source operating in the positive ion mode (Thermo Fisher Scientific; Waltham, MA). Separation was achieved using an Acquity UPLC HSS-T3 2.1×30 mm, 1.8 μM column (Waters Corporation, Milford, MA) at ambient temperature. A binary solvent system composed of 0.1% formic acid in H2O (mobile phase A) and 0.1% formic acid in acetonitrile (mobile phase B) was used for chromatographic separation. Selected reaction monitoring was used to quantify the analytes and internal standards, with the specific precursor to product transitions of: Arginine (344.96>70.14), [¹³C6]Arginine (351.102>74.3), Ornithine (473.29>171.15), [¹³C5]Ornithine (478.29>171.15). During analysis, samples were maintained at 10° C. in the autosampler chamber. For injection, 12 μL of sample were over-filled into a 5 μL, sample loop. All results were analyzed using the response ratio of analyte peak area/internal standard peak area for data normalization.

### Example 6

### Affinity Measurement of Anti-Arg1 Antibodies to Human Arg1 by Surface Plasmon Resonance

Monomeric hArg1 was previously created by the mutagenesis of Arg308 found at the monomeric interfaces to an alanine residue (Mortier et al., Sci. Rep. 7, 1-9 (2017)). Arg308 is important for maintaining a salt bridge between each hArg1 monomer and mutation of this residue leads to monomerization of hArg1 and nearly 85% loss in enzymatic activity (Ibid.) We sought to explore the affinity changes of these antibodies when bound to monomeric hArg1 versus trimeric hArg1.

The binding affinities of anti-hArg1 antibodies to monomeric and trimeric hArg1 were measured by capturing human mouse chimeric antibodies on an anti-mouse IgG surface or human/humanized antibodies on an anti-human Fc surface on a Series S Sensor Chip CMS (Cytiva) using a Biacore T200 or Biacore 4000 biosensor (Cytiva). Immobilization and affinity measurements were performed in 10 mM HEPES, 150 mM NaCl, 0.05% v/v Surfactant P20, 3 mM Ethylenediaminetetraacetic acid (EDTA), pH 7.4 (Cytiva) at 25° C. The anti-mouse IgG and anti-human Fc capture antibodies were immobilized on all surfaces of the chip following Cytiva's Amine Coupling Kit, Mouse Antibody Capture Kit, and Human Antibody Capture Kit protocols. To measure the affinity of each interaction, the anti-hArg1 antibodies were captured at 2 nM at 10 μL/minutes and 5-6 concentrations of a two-fold dilution of hArg1 from 100 nM were injected for three minutes at 30 or 50 μL/min. A reference flow cell without captured antibody was also included. After the antigen injections, the dissociation of the interaction was monitored for 5 or 10 minutes. The antibody and antigen were removed from the chip with a 30 second injection of 10 mM glycine at pH 1.5 for anti-mouse IgG captures or 3 M MgCl₂ for anti-human Fc captures between binding cycles.

The data were processed and fit using Biacore T200 Evaluation Software version 2.0 or Biacore 4000 Evaluation Software version 1.1 (Cytiva). The data were “double referenced” by subtracting the response from the reference control flow cell and that from a buffer injection. The data were then fit with the ‘1:1 Binding’ model to determine the association rate constant, ka (M⁻¹s⁻¹, where “M” equals molar and “s” equals seconds) and the dissociation rate constant, kd (s⁻¹). These rate constants were used to calculate the equilibrium dissociation constant, KD (M)=kd/ka.

The affinity measurements of mAb4 binding to both trimeric and monomeric hArg1 were determined by surface plasmon resonance (SPR) studies to gain insight into how hArg1 oligomerization corresponds to antibody binding. The affinity of mAb4 for hArg1 differed only by about 36 fold between trimeric hArg1 (0.56 nM) versus monomeric hArg1 (20 nM) (Table 8).

TABLE 8
SPR data for mAb4 affinitiy for monomeric and trimeric hArg1
hArg1 Trimeric

| Antibody | Ka (1/Ms) | Kd (1/s) | KD (M) | STD KD (M) |
|---|---|---|---|---|
| mAb4 | 2.9 × 10E5 | 1.6 × 10E−4 | 0.56 × 10E−9 | 5.7 × 10E−11 |

hArg1 monomeric

| Antibody | Ka (1/Ms) | Kd (1/s) | KD (M) | STD KD (M) |
|---|---|---|---|---|
| mAb4 | 3.9 × 10E5 | 7.6 × 10E−3 | 20.0 × 10E−9 | 6.8 × 10E−11 |

### Example 7

Antibody Binding Affinities to Trimeric hArg1 and Monomeric hArg1

The antibodies have interactions spanning across the hArg1 monomeric interfaces when hArg1 is present in the natural, trimeric form. SPR assays revealed the reduction or loss of binding potencies of the mAbs when hArg1 was forced into a monomeric state.

MAb4 binds across two monomers and the affinity of mAb4 for hArg1 differed only by about 36 fold when hArg1 was monomerized. Upon monomerization, mAb4 may have the ability to maintain binding to two separate hArg1 monomers. Within one hArg1 monomer, mAb4 shares 974 A² of surface area and two salt bridges while with the other monomer, mAb4 shares only 576 A² but has five salt bridges with hArg1 (Table 9). Despite having about 1.5-fold less surface area overlap in one of these mAb4:hArg1 pairs, the addition of several salt bridges may be enough to maintain binding.

TABLE 9
Surface area, hydrogen bonds, and salt
bridges between antibodies and hArg1

| | hArg1 | Antibody | Surface area | # Hydrogen | # salt |
|---|---|---|---|---|---|
| Antibody | monomer | chain | (Å²) | bonds | bridges |
| mAb4 | MonA | HC | 604 | 4 | 1 |
| | MonA | LC | 370 | 2 | 1 |
| | MonB | LC | 531 | 3 | 5 |
| | MonB | HC | 45 | 0 | 0 |

Surface area calculations done with proximal isovelocity surface area (PISA) (Krissinel & Henrick, J. Mol. Biol. 372, 774-797 (2007))

### Example 8

### Isothermal Titration Calorimetry Assay

Isothermal titration calorimetry (ITC) was conducted using a MicroCal PEAQ-ITC Automated (Malvern Inc, Westborough, MA) to determine the stoichiometry and thermodynamics properties of the hArg1:mAb interaction. Human Arg1 trimer and mAb4 (mouse/human chimera) were prepared in 10 mM HEPES pH 7.4 buffer containing 150 mM NaCl and 1 mM MnCl₂. Arginase (100 μM) in the ITC syringe was titrated into the antibody (10 μM) in the ITC cell at 25° C. Reference power was set to 10 μcal/sec with initial delay of 60 seconds and stirring speed of 750 rpm. Injection volume was 0.4 μL for the first injection and 4 μL for subsequent injections. Injection duration was 0.8 seconds for the first injection and 6 seconds for subsequent injections with 150 sec spacing. Baseline was adjusted using buffer-buffer titration. Data analysis was done using MicroCal PEAQ-ITC Analysis Software.

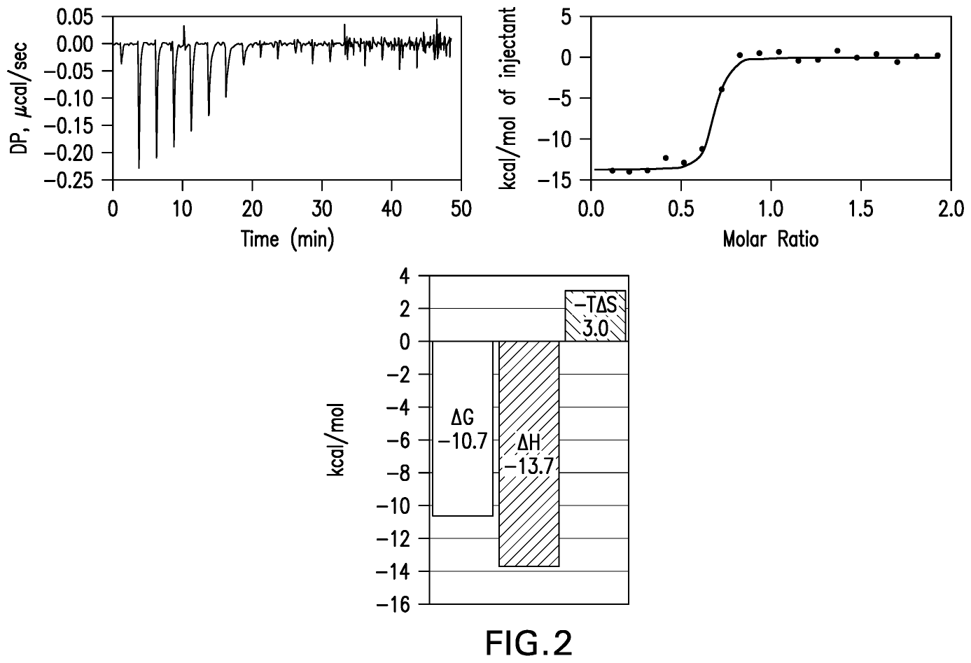

FIG. 2 shows the ITC results in which the calculated stoichiometry of n=0.637 is consistent with a 2:3 ratio of [hArg1]:[mAb].

### Example 9

### Cryo-Electron Microscopy Methods and Image Processing

All the hArg1:mAb complexes were formed by mixing the protein and the mAb in reaction buffer (25 mM HEPES pH 7.4, 150 mM NaCl, 1.0 mM MnCl₂) at 3:1 molar ratio and incubating the mixture for 30 minutes before preparing the grids. Grids were prepared and data were collected at NanoImaging Services (San Diego, CA) according to the following protocol and the specifications in Tables 10 and 11: After incubation, the sample containing the complex was diluted with reaction buffer to approx. Then, 75 μM concentration of monomeric Arginase was then mixed with dodecyl maltoside (DDM) to the critical micelle concentration (CMC) to reduce particle aggregation and used immediately afterwards to freeze grids. Then, 3 μL of each sample were applied to 1.2/1.3 grids (Au/Au Quantifoil or C/Cu C-flat), which have been previously plasma-cleaned using a Gatan Solarus (Pleasanton, California) and mounted in a Vitrobot Mark IV. The sample was then blotted with filter paper for six seconds and plunged in liquid ethane. Electron microscopy was performed using a ThermoFisher Titan Krios (Hillsboro, Oregon) transmission electron microscope operated at 300 kV and equipped with a Gatan Quantum 967 LS imaging filter and Gatan K2 Summit direct detector. Automated data-collection was carried out using Leginon software (Suloway et al., J. Struct. Biol. 151: 41-60 (2005)) in counting mode, collecting between 1500 and 3000 movies per sample at a defocus range between −1.0 and −2.0 μm, calibrated pixel size of 1.04 Å/pix and total dose of 45 e⁻/Å (Suloway et al., Ibid.).

Movies were aligned using MotionCor2 (Zheng et al., Nat. Methods14: 331-332 (2017)) and the contrast transfer function (CTF) calculated using CTFFIND4 ((Rohou. & Grigorieff, J. Struct. Biol. 192: 216-221 (2015)) within the Appion package (Lander et al., J. Struct. Biol.166: 95-102 (2009)). Aligned micrographs were imported into cryoSPARC (Punjani et al., Nat. Methods 14: 290-296 (2017)) where all the subsequent steps of image processing were realized following a standard single particle workflow until the final reconstruction. Several rounds of 2D classification ab-initio were performed to select the best-looking particles and separate the different oligomerization complexes when they existed. In all the cases non-uniform refinement and higher-order CTF refinement were used to generate the best reconstructions. Symmetries C3 or C1 were enforced during the final refinement when applicable. When used during the refinement, masks were generated in Chimera (Pettersen et al., J Comput Chem. 25:1605-12 (2004)). Initial models for the structures were generated using MOE 2018.01 (Chemical Computing Group ULC, Montreal, QC, Canada) and the available structure of hArg (PDB ID=6V7C) (Mitcheltree. et al., ACS Med. Chem. Lett. 11, 582-588 (2020)). The structures were built in Coot (Emsley et al., Biol. Crystallog. 60: 2126-2132 (2004)) based on the cryoEM density maps and subjected to one round of real space refinement in Phenix (Afonine et al., Comput. Crystallogr. Newsl. 4: 43-44 (2013)). Summary of cryo-EM data collection is shown in Table 10 and statistics data processing, map generation, and model refinement values are shown Table 11. Table 12 summarizes the specific epitope-paratope interactions between hArg1 and mAb4.

TABLE 10
Summary of cryo-EM data collection

| Sample | mAb4 |
|---|---|
| Microscope | Krios/EF |
| Camera | K2 |
| Pixel size (Å/pix) | 1.04 |
| Dose (e⁻/Å) | 44.47 |
| #images | 166 |
| Defocus range | 1.0 to −2.0 |
| (μm) | |

TABLE 11
Statistics data processing, map generation and model refinement

| | | mAb4 - full |
|---|---|---|
| | mAb4 -HiRes | Full Complex |
| | One trimer: three | (Two trimers: three |
| Sample | Fabs | mAbs) |
| #particles | 31797 | 31797 |
| Global resolution (Å) | 3.5 | 3.7 |
| RMSD Bonds (Å) | 0.011 | 0.007 |
| RMSD Angles (Deg) | 1.12 | 1.00 |
| CC Mask | 0.87 | 0.67 |
| CC volume | 0.87 | 0.68 |
| CC peaks | 0.58 | 0.63 |
| Ramachandran | | |
| Outliers (%) | 0.39 | 0.14 |
| Allowed (%) | 11.2 | 10.9 |
| Favored (%) | 88.4 | 89.0 |
| All-atom clashscore | 0.54 | 11.2 |

TABLE 12
Amino acid interactions between the
mAb4 paratope and the hArg1 epitope.

| Light Chain | hArg1 monomer A |
|---|---|
| Asn31 | Glu42 |
| Asp32 | Lys39 |
| Lys49 | Glu38 |
| Tyr50 | Lys33, Ala34, Gly35, Glu38, |
| Gln53 | Glu38, Lys41 |
| Asn92 | Lys39, Glu287, Thr290, Asn294 |
| Heavy Chain | hArg1 monomer A |
| Thr30, | Glu26 |
| Tyr33 | Lys33, Pro280 |
| Val50, Ser52 | Pro286 |
| Tyr54 | Glu26, Ser281 |
| Asn55 | Gly283 |
| Gly57 | Lys284, Thr285, Pro286 |
| Thr59 | Pro286, Glu287 |
| Asp99, Leu100 | Lys33 |
| Tyr101 | Lys33, Ala34, Thr290, Val293 |
| Tyr102 | Glu38 |
| Light Chain | hArg1 monomer B |
| Glu1 | Arg21 |
| Val3 | Arg21 |
| Arg24 | Lys68 |
| Ser26 | Arg21 |
| Gln27 | Gly245, Pro247 |
| Arg28 | Asp181, Val182, Asp183, Pro184 |
| Asp70 | Thr136 |
| Trp94 | Lys284 |

All interactions shown here are within 4 Å.
VH and VL amino acid position numbers are as set forth in SEQ ID
NO: 3 and 5, respectively, and not according to Kabat.

### Example 10

mAb4 2:3 complexes and impact of the 2:3 complex on hArg1 inhibition

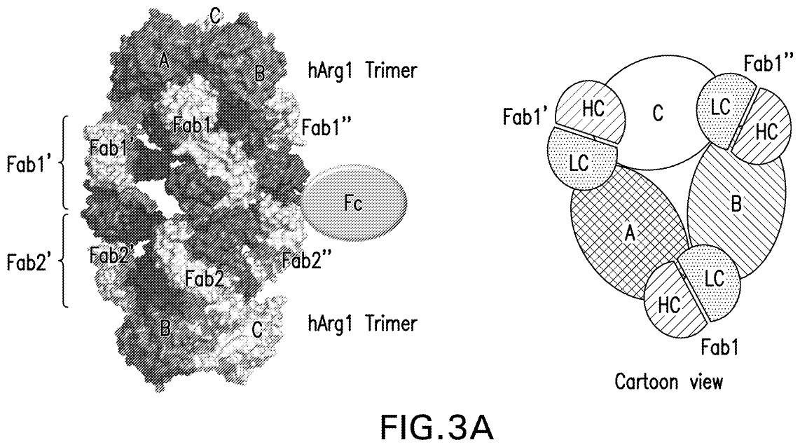

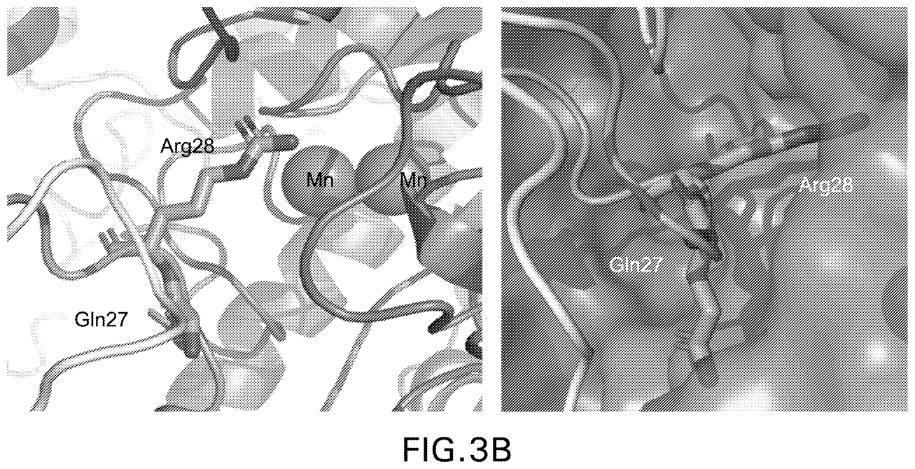

A mouse antibody campaign led to the discovery of mAb4, which was humanized and constructed on a human IgG4 S228P/kappa backbone. For each mAb4, the HC interacts with only one hArg1 monomers (FIG. 3A) while the LC interacts with both with two monomers. In this complex the mAb4 backbone is positioned so that the Fabs are at an approximately 120° angle. MAb4 binds directly over the opening to the active site thereby sterically blocking the hArg1 active site with Arg28 on the LC CDR-1 loop (FIG. 3B) inserted into and occluding the active site of hArg1, resulting in total inhibition of hArg1 activity. Additionally, Gln27 is positioned almost parallel to the surface of the active site channel, further sterically blocking substrate from entering. A complete list of the epitope and paratope interactions between hArg1 and mAb4 are summarized in Table 12.

The fully discernable formation of the large macromolecular complexes consisting of two trimers and three full mAbs is unique in the literature. Recently, a similar complex of a single HtrA1 trimer and three anti-HtrA1 Fabs was determined through negative staining EM (Ciferri et al., Biochem. J. 472, 169-181 (2015)) analyses coupled with a previously determined low-resolution SAXS structure of the HtrA1 trimer (Eigenbrot et al., Structure 20, 1040-1050 (2012)) to build a 1:3 model of enzyme:antibody. The full “cage-like” structure was proposed and generated by “juxtaposing and mirroring” two of the HtrA1:Fab 1:3 complexes resulting in a 2:3 HtrA1:Ab complex much like those presented here. In vitro enzymatic assays determined that anti-HtrA1 full-length antibodies had greater than 10-fold higher potency versus Fabs alone, hinting that the ability of HtrA1 to form these large macromolecular complexes of two HtrA1 trimers to three antibodies is instilling higher potency. While singular anti-hArg1 Fab arms were not tested in our studies, we hypothesize that mAb4 showing distinct 2:3 complexes is highly potent due in part to the complexation of enzymes and antibodies.

The means of inhibition by mAb4 is based on steric occlusion of the hArg1 active site. As shown in FIG. 3B and described previously, these antibodies function by inserting an amino acid side chain into, and therefore sterically blocking, the narrow active site channel. This is accomplished by Arg28 on the LC CDR-1 loop of mAb4.

Rather than relying on a specific residue for inhibition it is clear that overall steric occlusion of the active site is the inhibitory mechanism of mAb4.

### Example 11

### Comparison of Complex Sizes and Shapes

When considering the overall shape and size of the complexes (FIG. 4), it is clear that some of these differences are due to the varying backbone characteristics as described above. However, the epitope of the antibody is responsible for determining how and where the antibody binds to the hArg1 trimer which also plays a role in the antibody orientation and therefore the overall complex shape. Antibody mAb4 has mostly LC interactions with the hArg1 trimer and binds in such a way that the Fab is splayed slightly outwards resulting in a shorter (˜195 Å long) complex with a visibly smaller angle in the mAb backbone and a more rounded appearance (FIG. 4).

TABLE of
Sequences

| SEQ | | |
|---|---|---|
| ID | | |
| NO: | Description | Sequence |
| 1 | Human arginase 1 | MSAKSRTIGIIGAPFSKGQPRGGVEEGPTVLRKAGLLEKLK |
| | | EQECDVKDYGDLPFADIPNDSPFQIVKNPRSVGKASEQLAG |
| | | KVAEVKKNGRISLVLGGDHSLAIGSISGHARVHPDLGVIWV |
| | | DAHTDINTPLTTTSGNLHGQPVSFLLKELKGKIPDVPGFSW |
| | | VTPCISAKDIVYIGLRDVDPGEHYILKTLGIKYFSMTEVDRL |
| | | GIGKVMEETLSYLLGRKKRPIHLSFDVDGLDPSFTPATGTP |
| | | VVGGLTYREGLYITEEIYKTGLLSGLDIMEVNPSLGKTPEEV |
| | | TRTVNTAVAITLACFGLAREGNHKPIDYLNPPK |
| 2 | VH1 (mouse) | EVQLVQSGAEVKKPGASVKVSCKASGYTFTDYYMNWVR |
| | | QAPGQGLEWIGVISPYNGGTTYNQKFKGKATLTVDKSTST |
| | | AYMELSSLRSEDTAVYYCVYDLYYFDYWGQGTLVTVSS |
| 3 | VH2 (humanized | EVQLQQSGPVLVKPGASVKMSCKASEYTFTDYYMNWVK |
| | VH1) | QSHGRSLEWIGVISPYNGGTTYNQKFKGKATLTVDKSSST |
| | | AYMELNSLTSEDSAVYYCVYDLYYFDYWGQGTTLTVSS |
| 4 | VL1 (mouse) | EIVLTQSPATLSLSPGERATLSCRASQRISNDLHWYQQKPG |
| | | QAPRLLIKYASQSISGIPSRFSGSGSGTDFTLTISSLEPEDFAV |
| | | YYCQQSNSWPRTFGGGTKLEIK |
| 5 | VL2 (humanized | NIVLTQSPATLSVTPGDSVSLSCRASQRISNDLHWYQQKSH |
| | VL1) | ESPRLLIKYASQSISGIPSRFSGSGSGTDFTLSINSVETEDFG |
| | | MYFCQQSNSWPRTFGGGTKLEIK |
| 6 | VH1-CDR1 and | DYYMN |
| | VH2-CDR1 Kabat | |
| | (mouse and | |
| | humanized) | |
| 7 | VH1-CDR2 and | VISPYNGGTTYNQKFKG |
| | VH2-CDR2 | |
| | Kabat/Kabat + | |
| | Chothia (mouse and | |
| | humanized) | |
| 8 | VH1-CDR3 and | DLYYFDY |
| | VH2-CDR3 | |
| | Kabat/Chothia/ | |
| | Kabat + Chothia/AbM | |
| | (mouse and | |
| | humanized) | |
| 9 | VH1-CDR1 Chothia | GYTFTDY |
| | (mouse) | |
| 10 | VH2-CDR1 Chothia | EYTFTDY |
| | (humanized) | |
| 11 | VH1-CDR2 and | SPYNGG |
| | VH2-CDR2 Chothia | |
| | (mouse and | |
| | humanized) | |
| 12 | VH1-CDR1 and | GYTFTDYYMN |
| | VH2-CDR1 | |
| | Kabat + Chothia/ | |
| | AbM (mouse) | |
| 13 | VH2-CDR1 | EYTFTDYYMN |
| | Kabat + Chothia/ | |
| | AbM (humanized) | |
| 14 | VH1-CDR2 and | VISPYNGGTT |
| | VH2-CDR2 AbM | |
| | (mouse and | |
| | humanized) | |
| 15 | VH1-CDR1 IMGT | GYTFTDYY |
| | (mouse) | |
| 16 | VH2-CDR1 IMGT | EYTFTDYY |
| | (humanized) | |
| 17 | VH1-CDR2 and | ISPYNGG |
| | VH2-CDR2 IMGT | |
| | (mouse and | |
| | humanized) | |
| 18 | VH1-CDR3 and | VYDLYYFDY |
| | VH2-CDR3 IMGT | |
| | (mouse and | |
| | humanized) | |
| 19 | VH1-CDR1 and | TDYYMN |
| | VH2-CDR1 | |
| | Contact (mouse and | |
| | humanized) | |
| 20 | VH1-CDR2 and | WIGVISPYNGGTT |
| | VH2-CDR2 Contact | |
| | (mouse and | |
| | humanized) | |
| 21 | VH1-CDR3 and | VYDLYYFD |
| | VH2-CDR3 Contact | |
| | (mouse and | |
| | humanized) | |
| 22 | VL1-CDR1 and | RASQRISNDLH |
| | VL2-CDR1 | |
| | Kabat/Chothia/ | |
| | Kabat + Chothia/AbM | |
| | (mouse and | |
| | humanized) | |
| 23 | VL1-CDR2 and | YASQSIS |
| | VL2-CDR2 | |
| | Kabat/Chothia/ | |
| | Kabat + Chothia/AbM | |
| | (mouse and | |
| | humanized) | |
| 24 | VL1-CDR3 and | QQSNSWPRT |
| | VL2-CDR3 | |
| | Kabat/Chothia/ | |
| | Kabat + Chothia/AbM/ | |
| | IMGT (mouse and | |
| | humanized) | |
| 25 | VL1-CDR1 and | QRISND |
| | VL2-CDR1 IMGT | |
| | (mouse and | |
| | humanized) | |
| 26 | VL1-CDR1 and | SNDLHWY |
| | VL2-CDR1 Contact | |
| | (mouse and | |
| | humanized) | |
| 27 | VL1-CDR2 and | LLIKYASQSI |
| | VL2-CDR2 Contact | |
| | (mouse and | |
| | humanized) | |
| 28 | VL1-CDR3 and | QQSNSWPR |
| | VL2-CDR3 Contact | |
| | (mouse and | |
| | humanized) | |
| 29 | Human IgG1 HC | ASTKGPSVFPLAPSSKSTSGGTAALGCLVKDYFPEPVTVSW |
| | constant domain | NSGALTSGVHTFPAVLQSSGLYSLSSVVTVPSSSLGTQTYIC |
| | | NVNHKPSNTKVDKKVEPKSCDKTHTCPPCPAPELLGGPSVF |
| | | LFPPKPKDTLMISRTPEVTCVVVDVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 30 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (L234A L235A | NVNHKPSNTKVDKKVEPKSCDKTHTCPPCPAPEAAGGPSV |
| | D265S) | FLFPPKPKDTLMISRTPEVTCVVVSVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 31 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (L234A L235A | NVNHKPSNTKVDKKVEPKSCDKTHTCPPCPAPEAAGGPSV |
| | P329G) | FLFPPKPKDTLMISRTPEVTCVVVSVSHEDPEVKFNWYVDG |
| | | VEVHNAKTKPREEQYNSTYRVVSVLTVLHQDWLNGKEYK |
| | | CKVSNKALGAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 32 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (L235E) | NVNHKPSNTKVDKKVEPKSCDKTHTCPPCPAPELEGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| 33 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (D265A) | NVNHKPSNTKVDKKVEPKSCDKTHTCPPCPAPELLGGPSVF |
| | | LFPPKPKDTLMISRTPEVTCVVVAVSHEDPEVKFNWYVDG |
| | | VEVHNAKTKPREEQYNSTYRVVSVLTVLHQDWLNGKEYK |
| | | CKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 34 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (D265A N297G) | NVNHKPSNTKVDKKVEPKSCDKTHTCPPCPAPELLGGPSVF |
| | | LFPPKPKDTLMISRTPEVTCVVVAVSHEDPEVKFNWYVDG |
| | | VEVHNAKTKPREEQYGSTYRVVSVLTVLHQDWLNGKEYK |
| | | CKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 35 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (E233A/L235A) | NVNHKPSNTKVDKKVEPKSCDKTHTCPPCPAPALAGGPSV |
| | | FLFPPKPKDTLMISRTPEVTCVVVDVSHEDPEVKFNWYVD |
| | | GVEVHNAKTKPREEQYNSTYRVVSVLTVLHQDWLNGKEY |
| | | KCKVSNKALPAPIEKTISKAKGQPREPQVYTLPPSRDELTK |
| | | NQVSLTCLVKGFYPSDIAVEWESNGQPENNYKTTPPVLDS |
| | | DGSFFLYSKLTVDKSRWQQGNVFSCSVMHEALHNHYTQK |
| | | SLSLSPGK |
| 36 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (N297X, wherein X | NVNHKPSNTKVDKKVEPKSCDKTHTCPPCPAPELLGGPSVF |
| | is any amino acid | LFPPKPKDTLMISRTPEVTCVVVDVSHEDPEVKFNWYVDG |
| | other than N) | VEVHNAKTKPREEQYXSTYRVVSVLTVLHQDWLNGKEYK |
| | | CKVSNKALPAPIEKTISKAKGQPREPQVYTLPPSRDELTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 37 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | | CNVDHKPSNTKVDKTVERKCCVECPPCPAPPVAGPSVFLFP |
| | | PKPKDTLMISRTPEVTCVVVDVSHEDPEVQFNWYVDGVEV |
| | | HNAKTKPREEQFNSTFRVVSVLTVVHQDWLNGKEYKCKV |
| | | SNKGLPAPIEKTISKTKGQPREPQVYTLPPSREEMTKNQVSL |
| | | TCLVKGFYPSDISVEWESNGQPENNYKTTPPMLDSDGSFFL |
| | | YSKLTVDKSRWQQGNVFSCSVMHEALHNHYTQKSLSLSP |
| | | GK |
| 38 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (D265S) | CNVDHKPSNTKVDKTVERKCCVECPPCPAPPVAGPSVFLFP |
| | | PKPKDTLMISRTPEVTCVVVSVSHEDPEVQFNWYVDGVEV |
| | | HNAKTKPREEQFNSTFRVVSVLTVVHQDWLNGKEYKCKV |
| | | SNKGLPAPIEKTISKTKGQPREPQVYTLPPSREEMTKNQVSL |
| | | TCLVKGFYPSDISVEWESNGQPENNYKTTPPMLDSDGSFFL |
| | | YSKLTVDKSRWQQGNVFSCSVMHEALHNHYTQKSLSLSP |
| | | GK |
| 39 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (P329G) | CNVDHKPSNTKVDKTVERKCCVECPPCPAPPVAGPSVFLFP |
| | | PKPKDTLMISRTPEVTCVVVDVSHEDPEVQFNWYVDGVEV |
| | | HNAKTKPREEQFNSTFRVVSVLTVVHQDWLNGKEYKCKV |
| | | SNKGLGAPIEKTISKTKGQPREPQVYTLPPSREEMTKNQVS |
| | | LTCLVKGFYPSDISVEWESNGQPENNYKTTPPMLDSDGSFF |
| | | LYSKLTVDKSRWQQGNVFSCSVMHEALHNHYTQKSLSLSP |
| | | GK |
| 40 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (D265A) | CNVDHKPSNTKVDKTVERKCCVECPPCPAPPVAGPSVFLFP |
| | | PKPKDTLMISRTPEVTCVVVAVSHEDPEVQFNWYVDGVEV |
| | | HNAKTKPREEQFNSTFRVVSVLTVVHQDWLNGKEYKCKV |
| | | SNKGLPAPIEKTISKTKGQPREPQVYTLPPSREEMTKNQVSL |
| | | TCLVKGFYPSDISVEWESNGQPENNYKTTPPMLDSDGSFFL |
| | | YSKLTVDKSRWQQGNVFSCSVMHEALHNHYTQKSLSLSP |
| | | GK |
| 41 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (D265A N297G) | CNVDHKPSNTKVDKTVERKCCVECPPCPAPPVAGPSVFLFP |
| | | PKPKDTLMISRTPEVTCVVVAVSHEDPEVQFNWYVDGVEV |
| | | HNAKTKPREEQFGSTFRVVSVLTVVHQDWLNGKEYKCKV |
| | | SNKGLPAPIEKTISKTKGQPREPQVYTLPPSREEMTKNQVSL |
| | | TCLVKGFYPSDISVEWESNGQPENNYKTTPPMLDSDGSFFL |
| | | YSKLTVDKSRWQQGNVFSCSVMHEALHNHYTQKSLSLSP |
| | | GK |
| 42 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (N297X, wherein X | CNVDHKPSNTKVDKTVERKCCVECPPCPAPPVAGPSVFLFP |
| | is any amino acid | PKPKDTLMISRTPEVTCVVVDVSHEDPEVQFNWYVDGVEV |
| | other than N) | HNAKTKPREEQFXSTFRVVSVLTVVHQDWLNGKEYKCKV |
| | | SNKGLPAPIEKTISKTKGQPREPQVYTLPPSREEMTKNQVSL |
| | | TCLVKGFYPSDISVEWESNGQPENNYKTTPPMLDSDGSFFL |
| | | YSKLTVDKSRWQQGNVFSCSVMHEALHNHYTQKSLSLSP |
| | | GK |
| 43 | Human IgG2 HC | ASTKGPSVFPLAPCSRSTSESTAALGCLVKDYFPEPVTVSW |
| | Constant domain | NSGALTSGVHTFPAVLQSSGLYSLSSVVTVPSSNFGTQTYT |
| | (V234A G237A | CNVDHKPSNTKVDKTVERKCCVECPPCPAPPAAASSVFLFP |
| | P238S H268A | PKPKDTLMISRTPEVTCVVVDVSAEDPEVQFNWYVDGVEV |
| | V309L A330S | HNAKTKPREEQFNSTFRVVSVLTVLHQDWLNGKEYKCKV |
| | P331S | SNKGLPSSIEKTISKTKGQPREPQVYTLPPSREEMTKNQVSL |
| | X378S/A)(See | TCLVKGFYPSDIXVEWESNGQPENNYKTTPPMLDSDGSFFL |
| | IgGsigma SEQ ID | YSKLTVDKSRWQQGNVFSCSVMHEALHNHYTQKSLSLSP |
| | No: 78 in | GK |
| | WO2017079112) | |
| 44 | Human IgG4 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (S228P) | CNVDHKPSNTKVDKRVESKYGPPCPPCPAPEFLGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| 45 | Human IgG4 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (S228P P329G) | CNVDHKPSNTKVDKRVESKYGPPCPPCPAPEFLGGPSVFLF |
| | | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLGSSIEKTISKAKGQPREPQVYTLPPSQEEMTKNQV |
| | | SLTCLVKGFYPSDIAVEWESNGQPENNYKTTPPVLDSDGSF |
| | | FLYSRLTVDKSRWQEGNVFSCSVMHEALHNHYTQKSLSLS |
| | | LGK |
| 46 | Human IgG4 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (S228P D265A) | CNVDHKPSNTKVDKRVESKYGPPCPPCPAPEFLGGPSVFLF |
| | | PPKPKDTLMISRTPEVTCVVVAVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| 47 | Human IgG4 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (S228P D265A | CNVDHKPSNTKVDKRVESKYGPPCPPCPAPEFLGGPSVFLF |
| | N297G) | PPKPKDTLMISRTPEVTCVVVAVSQEDPEVQFNWYVDGVE |
| | | VHNAKTKPREEQFGSTYRVVSVLTVLHQDWLNGKEYKCK |
| | | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| 48 | Human IgG4 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (S228P N297X, | CNVDHKPSNTKVDKRVESKYGPPCPPCPAPEFLGGPSVFLF |
| | wherein X is any | PPKPKDTLMISRTPEVTCVVVDVSQEDPEVQFNWYVDGVE |
| | amino acid other | VHNAKTKPREEQFXSTYRVVSVLTVLHQDWLNGKEYKCK |
| | than N) | VSNKGLPSSIEKTISKAKGQPREPQVYTLPPSQEEMTKNQVS |
| | | LTCLVKGFYPSDIAVEWESNGQPENNYKTTPPVLDSDGSFF |
| | | LYSRLTVDKSRWQEGNVFSCSVMHEALHNHYTQKSLSLSL |
| | | GK |
| 49 | Human LC Kappa | RTVAAPSVFIFPPSDEQLKSGTASVVCLLNNFYPREAKVQW |
| | Constant domain | KVDNALQSGNSQESVTEQDSKDSTYSLSSTLTLSKADYEK |
| | | HKVYACEVTHQGLSSPVTKSFNRGEC |
| 50 | Human IgG1 HC | ASTKGPSVFPLAPSSKSTSGGTAALGCLVKDYFPEPVTVSW |
| | Constant domain | NSGALTSGVHTFPAVLQSSGLYSLSSVVTVPSSSLGTQTYIC |
| | (N297A/D356E/L358M) | NVNHKPSNTKVDKKVEPKSCDKTHTCPPCPAPELLGGPSVF |
| | | LFPPKPKDTLMISRTPEVTCVVVDVSHEDPEVKFNWYVDG |
| | | VEVHNAKTKPREEQYASTYRVVSVLTVLHQDWLNGKEYK |
| | | CKVSNKALPAPIEKTISKAKGQPREPQVYTLPPSREEMTKN |
| | | QVSLTCLVKGFYPSDIAVEWESNGQPENNYKTTPPVLDSD |
| | | GSFFLYSKLTVDKSRWQQGNVFSCSVMHEALHNHYTQKS |
| | | LSLSPGK |
| 51 | Human IgG1 HC | ASTKGPSVFPLAPCSRSTSESTAALGCLVKDYFPEPVTVSW |
| | Constant domain | NSGALTSGVHTFPAVLQSSGLYSLSSVVTVPSSSLGTKTYT |
| | (L234F/L235E/P331S/ | CNVDHKPSNTKVDKRVESKYGPPCPPCPAPEFLGGPSVFLF |
| | D356E/L358M) | PPKPKDTLMISRTPEVTCVVVDVSQEDPEVQFNWYVDGVE |
| | | VHNAKTKPREEQFNSTYRVVSVLTVLHQDWLNGKEYKCK |
| | | VSNKGLPSSIEKTISKAKGQPREPQVYTLPPSLTCLVKGFYP |
| | | SDIAVEWESNGQPENNYKTTPPVLDSDGSFFLYSRLTVDKS |
| | | RWQEGNVFSCSVMHEALHNHYTQKSLSLSLGK |
| 52 | Human LC lambda | GQPKANPTVTLFPPSSEELQANKATLVCLISDFYPGAVTVA |
| | Constant domain | WKADGSPVKAGVETTKPSKQSNNKYAASSYLSLTPEQWK |
| | | SHRSYSCQVTHEGSTVEKTVAPTECS |
| 53 | Mouse HC IgG1 | AKTTPPSVYPLAPGSAAQTNSMVTLGCLVKGYFPEPVTVT |
| | (D265A) constant | WNSGSLSSGVHTFPAVLQSDLYTLSSSVTVPSSTWPSETVT |
| | domain | CNVAHPASSTKVDKKIVPRDCGCKPCICTVPEVSSVFIFPPK |
| | | PKDVLTITLTPKVTCVVVAISKDDPEVQFSWFVDDVEVHTA |
| | | QTQPREEQFNSTFRSVSELPIMHQDWLNGKEFKCRVNSAAF |
| | | PAPIEKTISKTKGRPKAPQVYTIPPPKEQMAKDKVSLTCMIT |
| | | DFFPEDITVEWQWNGQPAENYKNTQPIMDTDGSYFVYSKL |
| | | NVQKSNWEAGNTFTCSVLHEGLHNHHTEKSLSHSPGK |
| 54 | Mouse HC IgG2 | AKTTAPSVYPLAPVCGDTTGSSVTLGCLVKGYFPEPVTLT |
| | constant domain | WNSGSLSSGVHTFPAVLQSDLYTLSSSVTVTSSTWPSQSITC |
| | | NVAHPASSTKVDKKIEPRGPTIKPCPPCKCPAPNLLGGPSVF |
| | | IFPPKIKDVLMISLSPIVTCVVVDVSEDDPDVQISWFVNNVE |
| | | VHTAQTQTHREDYNSTLRVVSALPIQHQDWMSGKEFKCK |
| | | VNNKDLPAPIERTISKPKGSVRAPQVYVLPPPEEEMTKKQV |
| | | TLTCMVTDFMPEDIYVEWTNNGKTELNYKNTEPVLDSDGS |
| | | YFMYSKLRVEKKNWVERNSYSCSVVHEGLHNHHTTKSFS |
| | | RTPGK |
| | Mouse LC kappa | RTVAAPTVSIFPPSSEQLTSGGASVVCFLNNFYPKDINVKW |
| | constant domain | KIDGSERQNGVLNSWTDQDSKDSTYSMSSTLTLTKDEYER |
| | | HNSYTCEATHKTSTSPIVKSFNRNEC |
| 55 | Nucleotide sequence | GAGGTGCAGCTGGTGCAGAGCGGCGCCGAGGTGAAGAA |
| | encoding VH1 | GCCCGGCGCCAGCGTGAAGGTGAGCTGCAAGGCCAGCG |
| | (mouse) | GCTACACCTTCACCGACTACTACATGAACTGGGTGAGAC |
| | | AGGCCCCCGGCCAGGGCCTGGAGTGGATCGGCGTGATC |
| | | AGCCCCTACAACGGCGGCACCACCTACAACCAGAAGTTC |
| | | AAGGGCAAGGCCACCCTGACCGTGGACAAGAGCACCAG |
| | | CACCGCCTACATGGAGCTGAGCAGCCTGAGAAGCGAGG |
| | | ACACCGCCGTGTACTACTGCGTGTACGACCTGTACTACT |
| | | TCGACTACTGGGGCCAGGGCACCCTGGTGACCGTGAGCA |
| | | GC |
| 56 | Nucleotide sequence | GAGGTGCAGCTGCAGCAGAGCGGCCCCGTGCTGGTGAA |
| | encoding VH2 | GCCCGGCGCCAGCGTGAAGATGAGCTGCAAGGCCAGCG |
| | (humanized VH1) | AGTACACCTTCACCGACTACTACATGAACTGGGTGAAGC |
| | | AGAGCCACGGCAGAAGCCTGGAGTGGATCGGCGTGATC |
| | | AGCCCCTACAACGGCGGCACCACCTACAACCAGAAGTTC |
| | | AAGGGCAAGGCCACCCTGACCGTGGACAAGAGCAGCAG |
| | | CACCGCCTACATGGAGCTGAACAGCCTGACCAGCGAGG |
| | | ACAGCGCCGTGTACTACTGCGTGTACGACCTGTACTACT |
| | | TCGACTACTGGGGCCAGGGCACCACCCTGACCGTGAGCA |
| | | GC |
| 57 | Nucleotide sequence | GAGATCGTGCTGACCCAGAGCCCCGCCACCCTGAGCCTG |
| | encoding VL1 | AGCCCCGGCGAGAGAGCCACCCTGAGCTGCAGAGCCAG |
| | (mouse) | CCAGAGAATCAGCAACGACCTGCACTGGTACCAGCAGA |
| | | AGCCCGGCCAGGCCCCCAGACTGCTGATCAAGTACGCCA |
| | | GCCAGAGCATCAGCGGCATCCCCAGCAGATTCAGCGGC |
| | | AGCGGCAGCGGCACCGACTTCACCCTGACCATCAGCAGC |
| | | CTGGAGCCCGAGGACTTCGCCGTGTACTACTGCCAGCAG |
| | | AGCAACAGCTGGCCCAGAACCTTCGGCGGCGGCACCAA |
| | | GCTGGAGATCAAG |
| 58 | Nucleotide sequence | AACATCGTGCTGACCCAGAGCCCCGCCACCCTGAGCGTG |
| | encoding VL2 | ACCCCCGGCGACAGCGTGAGCCTGAGCTGCAGAGCCAG |
| | (humanized VL1) | CCAGAGAATCAGCAACGACCTGCACTGGTACCAGCAGA |
| | | AGAGCCACGAGAGCCCCAGACTGCTGATCAAGTACGCC |
| | | AGCCAGAGCATCAGCGGCATCCCCAGCAGATTCAGCGG |
| | | CAGCGGCAGCGGCACCGACTTCACCCTGAGCATCAACA |
| | | GCGTGGAGACCGAGGACTTCGGCATGTACTTCTGCCAGC |
| | | AGAGCAACAGCTGGCCCAGAACCTTCGGCGGCGGCACC |
| | | AAGCTGGAGATCAAG |
| 59 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC constant | CTGCCTGGTGAAGGACTACTTCCCCGAGCCCGTGACCGT |
| | domain | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| 60 | Nucleotide sequence | CGACAAGACCCACACCTGCCCCCCCTGCCCCGCCCCCGA |
| | encoding human | GCTGCTGGGCGGCCCCAGCGTGTTCCTGTTCCCCCCCAA |
| | IgG1 HC Constant | GCCCAAGGACACCCTGATGATCAGCAGAACCCCCGAGG |
| | domain (L234A | TGACCTGCGTGGTGGTGGACGTGAGCCACGAGGACCCC |
| | L235A D265S) | GAGGTGAAGTTCAACTGGTACGTGGACGGCGTGGAGGT |
| | | GCACAACGCCAAGACCAAGCCCAGAGAGGAGCAGTACA |
| | | ACAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 61 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain (L234A | GAGCTGGAACAGCGGCGCCCTGACCAGCGGCGTGCACA |
| | L235A P329G) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GGCCGCCGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGAGCGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACA |
| | | ACAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 62 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain (L235E) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GGCCGCCGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGAGCGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACA |
| | | ACAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGGGCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 63 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain (D265A) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GCTGGAGGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGACGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACA |
| | | ACAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 64 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain (D265A | GAGCTGGAACAGCGGCGCCCTGACCAGCGGCGTGCACA |
| | N297G) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GCTGCTGGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGCCGTGAGCCACGAGGACCCCG |
| | | AGGTGAAGTTCAACTGGTACGTGGACGGCGTGGAGGTG |
| | | CACAACGCCAAGACCAAGCCCAGAGAGGAGCAGTACAA |
| | | CAGCACCTACAGAGTGGTGAGCGTGCTGACCGTGCTGCA |
| | | CCAGGACTGGCTGAACGGCAAGGAGTACAAGTGCAAGG |
| | | TGAGCAACAAGGCCCTGCCCGCCCCCATCGAGAAGACC |
| | | ATCAGCAAGGCCAAGGGCCAGCCCAGAGAGCCCCAGGT |
| | | GTACACCCTGCCCCCCAGCAGAGACGAGCTGACCAAGA |
| | | ACCAGGTGAGCCTGACCTGCCTGGTGAAGGGCTTCTACC |
| | | CCAGCGACATCGCCGTGGAGTGGGAGAGCAACGGCCAG |
| | | CCCGAGAACAACTACAAGACCACCCCCCCCGTGCTGGAC |
| | | AGCGACGGCAGCTTCTTCCTGTACAGCAAGCTGACCGTG |
| | | GACAAGAGCAGATGGCAGCAGGGCAACGTGTTCAGCTG |
| | | CAGCGTGATGCACGAGGCCCTGCACAACCACTACACCCA |
| | | GAAGAGCCTGAGCCTGAGCCCCGGCAAG |
| 65 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain | GAGCTGGAACAGCGGCGCCCTGACCAGCGGCGTGCACA |
| | (E233A/L235A) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GCTGCTGGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGCCGTGAGCCACGAGGACCCCG |
| | | AGGTGAAGTTCAACTGGTACGTGGACGGCGTGGAGGTG |
| | | CACAACGCCAAGACCAAGCCCAGAGAGGAGCAGTACGG |
| | | CAGCACCTACAGAGTGGTGAGCGTGCTGACCGTGCTGCA |
| | | CCAGGACTGGCTGAACGGCAAGGAGTACAAGTGCAAGG |
| | | TGAGCAACAAGGCCCTGCCCGCCCCCATCGAGAAGACC |
| | | ATCAGCAAGGCCAAGGGCCAGCCCAGAGAGCCCCAGGT |
| | | GTACACCCTGCCCCCCAGCAGAGACGAGCTGACCAAGA |
| | | ACCAGGTGAGCCTGACCTGCCTGGTGAAGGGCTTCTACC |
| | | CCAGCGACATCGCCGTGGAGTGGGAGAGCAACGGCCAG |
| | | CCCGAGAACAACTACAAGACCACCCCCCCCGTGCTGGAC |
| | | AGCGACGGCAGCTTCTTCCTGTACAGCAAGCTGACCGTG |
| | | GACAAGAGCAGATGGCAGCAGGGCAACGTGTTCAGCTG |
| | | CAGCGTGATGCACGAGGCCCTGCACAACCACTACACCCA |
| | | GAAGAGCCTGAGCCTGAGCCCCGGCAAG |
| 66 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain (N297X, | GAGCTGGAACAGCGGCGCCCTGACCAGCGGCGTGCACA |
| | wherein X is any | CCTTCCCCGCCGTGCTGCAGAGCAGCGGCCTGTACAGCC |
| | amino acid other | TGAGCAGCGTGGTGACCGTGCCCAGCAGCAGCCTGGGC |
| | than N); codon 538- | ACCCAGACCTACATCTGCAACGTGAACCACAAGCCCAGC |
| | 540 wherein N is | AACACCAAGGTGGACAAGAAGGTGGAGCCCAAGAGCTG |
| | any nucleotide with | CGACAAGACCCACACCTGCCCCCCCTGCCCCGCCCCCGC |
| | the proviso that the | CCTGGCCGGCGGCCCCAGCGTGTTCCTGTTCCCCCCCAA |
| | codon does not | GCCCAAGGACACCCTGATGATCAGCAGAACCCCCGAGG |
| | encode Asn | TGACCTGCGTGGTGGTGGACGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACA |
| | | ACAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 67 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG2 HC Constant | CTGCCTGGTGAAGGACTACTTCCCCGAGCCCGTGACCGT |
| | domain | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GCTGCTGGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGACGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACN |
| | | NNAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGACGAGCTGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 68 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (D265S) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAACTTCGGCA |
| | | CCCAGACCTACACCTGCAACGTGGACCACAAGCCCAGC |
| | | AACACCAAGGTGGACAAGACCGTGGAGAGAAAGTGCTG |
| | | CGTGGAGTGCCCCCCCTGCCCCGCCCCCCCCGTGGCCGG |
| | | CCCCAGCGTGTTCCTGTTCCCCCCCAAGCCCAAGGACAC |
| | | CCTGATGATCAGCAGAACCCCCGAGGTGACCTGCGTGGT |
| | | GGTGGACGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCAACAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGCCCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 69 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (P329G) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAACTTCGGCA |
| | | CCCAGACCTACACCTGCAACGTGGACCACAAGCCCAGC |
| | | AACACCAAGGTGGACAAGACCGTGGAGAGAAAGTGCTG |
| | | CGTGGAGTGCCCCCCCTGCCCCGCCCCCCCCGTGGCCGG |
| | | CCCCAGCGTGTTCCTGTTCCCCCCCAAGCCCAAGGACAC |
| | | CCTGATGATCAGCAGAACCCCCGAGGTGACCTGCGTGGT |
| | | GGTGAGCGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCAACAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGCCCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 70 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (D265A) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAACTTCGGCA |
| | | CCCAGACCTACACCTGCAACGTGGACCACAAGCCCAGC |
| | | AACACCAAGGTGGACAAGACCGTGGAGAGAAAGTGCTG |
| | | CGTGGAGTGCCCCCCCTGCCCCGCCCCCCCCGTGGCCGG |
| | | CCCCAGCGTGTTCCTGTTCCCCCCCAAGCCCAAGGACAC |
| | | CCTGATGATCAGCAGAACCCCCGAGGTGACCTGCGTGGT |
| | | GGTGGACGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCAACAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGGGCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 71 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (D265A | GAGCTGGAACAGCGGCGCCCTGACCAGCGGCGTGCACA |
| | N297G) | CCTTCCCCGCCGTGCTGCAGAGCAGCGGCCTGTACAGCC |
| | | TGAGCAGCGTGGTGACCGTGCCCAGCAGCAACTTCGGCA |
| | | CCCAGACCTACACCTGCAACGTGGACCACAAGCCCAGC |
| | | AACACCAAGGTGGACAAGACCGTGGAGAGAAAGTGCTG |
| | | CGTGGAGTGCCCCCCCTGCCCCGCCCCCCCCGTGGCCGG |
| | | CCCCAGCGTGTTCCTGTTCCCCCCCAAGCCCAAGGACAC |
| | | CCTGATGATCAGCAGAACCCCCGAGGTGACCTGCGTGGT |
| | | GGTGGCCGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCAACAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGCCCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 72 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (N297X, | GAGCTGGAACAGCGGCGCCCTGACCAGCGGCGTGCACA |
| | wherein X is any | CCTTCCCCGCCGTGCTGCAGAGCAGCGGCCTGTACAGCC |
| | amino acid other | TGAGCAGCGTGGTGACCGTGCCCAGCAGCAACTTCGGCA |
| | than N); codon 526- | CCCAGACCTACACCTGCAACGTGGACCACAAGCCCAGC |
| | 528 wherein N is | AACACCAAGGTGGACAAGACCGTGGAGAGAAAGTGCTG |
| | any nucleotide with | CGTGGAGTGCCCCCCCTGCCCCGCCCCCCCCGTGGCCGG |
| | the proviso that the | CCCCAGCGTGTTCCTGTTCCCCCCCAAGCCCAAGGACAC |
| | codon does not | CCTGATGATCAGCAGAACCCCCGAGGTGACCTGCGTGGT |
| | encode Asn | GGTGGCCGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCGGCAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGCCCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 73 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG2 HC Constant | CTGCCTGGTGAAGGACTACTTCCCCGAGCCCGTGACCGT |
| | domain (V234A | GAGCTGGAACAGCGGCGCCCTGACCAGCGGCGTGCACA |
| | G237A P238S | CCTTCCCCGCCGTGCTGCAGAGCAGCGGCCTGTACAGCC |
| | H268A V309L | TGAGCAGCGTGGTGACCGTGCCCAGCAGCAACTTCGGCA |
| | A330S P331S | CCCAGACCTACACCTGCAACGTGGACCACAAGCCCAGC |
| | X378S/A)(See | AACACCAAGGTGGACAAGACCGTGGAGAGAAAGTGCTG |
| | IgGsigma SEQ ID | CGTGGAGTGCCCCCCCTGCCCCGCCCCCCCCGTGGCCGG |
| | No: 78 in | CCCCAGCGTGTTCCTGTTCCCCCCCAAGCCCAAGGACAC |
| | WO2017079112) | CCTGATGATCAGCAGAACCCCCGAGGTGACCTGCGTGGT |
| | | GGTGGACGTGAGCCACGAGGACCCCGAGGTGCAGTTCA |
| | | ACTGGTACGTGGACGGCGTGGAGGTGCACAACGCCAAG |
| | | ACCAAGCCCAGAGAGGAGCAGTTCNNNAGCACCTTCAG |
| | | AGTGGTGAGCGTGCTGACCGTGGTGCACCAGGACTGGCT |
| | | GAACGGCAAGGAGTACAAGTGCAAGGTGAGCAACAAGG |
| | | GCCTGCCCGCCCCCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCA |
| | | GCGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 74 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG4 HC Constant | CTGCCTGGTGAAGGACTACTTCCCCGAGCCCGTGACCGT |
| | domain (S228P) | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAACTTCGGCA |
| | | CCCAGACCTACACCTGCAACGTGGACCACAAGCCCAGC |
| | | AACACCAAGGTGGACAAGACCGTGGAGAGAAAGTGCTG |
| | | CGTGGAGTGCCCCCCCTGCCCCGCCCCCCCCGCCGCCGC |
| | | CAGCAGCGTGTTCCTGTTCCCCCCCAAGCCCAAGGACAC |
| | | CCTGATGATCAGCAGAACCCCCGAGGTGACCTGCGTGGT |
| | | GGTGGACGTGAGCGCCGAGGACCCCGAGGTGCAGTTCA |
| | | ACTGGTACGTGGACGGCGTGGAGGTGCACAACGCCAAG |
| | | ACCAAGCCCAGAGAGGAGCAGTTCAACAGCACCTTCAG |
| | | AGTGGTGAGCGTGCTGACCGTGCTGCACCAGGACTGGCT |
| | | GAACGGCAAGGAGTACAAGTGCAAGGTGAGCAACAAGG |
| | | GCCTGCCCAGCAGCATCGAGAAGACCATCAGCAAGACC |
| | | AAGGGCCAGCCCAGAGAGCCCCAGGTGTACACCCTGCC |
| | | CCCCAGCAGAGAGGAGATGACCAAGAACCAGGTGAGCC |
| | | TGACCTGCCTGGTGAAGGGCTTCTACCCCAGCGACATCN |
| | | NNGTGGAGTGGGAGAGCAACGGCCAGCCCGAGAACAAC |
| | | TACAAGACCACCCCCCCCATGCTGGACAGCGACGGCAG |
| | | CTTCTTCCTGTACAGCAAGCTGACCGTGGACAAGAGCAG |
| | | ATGGCAGCAGGGCAACGTGTTCAGCTGCAGCGTGATGC |
| | | ACGAGGCCCTGCACAACCACTACACCCAGAAGAGCCTG |
| | | AGCCTGAGCCCCGGCAAG |
| 75 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG4 HC Constant | CTGCCTGGTGAAGGACTACTTCCCCGAGCCCGTGACCGT |
| | domain (S228P | GAGCTGGAACAGCGGCGCCCTGACCAGCGGCGTGCACA |
| | P329G) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCAAGACCTACACCTGCAACGTGGACCACAAGCCCAG |
| | | CAACACCAAGGTGGACAAGAGAGTGGAGAGCAAGTACG |
| | | GCCCCCCCTGCCCCCCCTGCCCCGCCCCCGAGTTCCTGG |
| | | GCGGCCCCAGCGTGTTCCTGTTCCCCCCCAAGCCCAAGG |
| | | ACACCCTGATGATCAGCAGAACCCCCGAGGTGACCTGCG |
| | | TGGTGGTGGACGTGAGCCAGGAGGACCCCGAGGTGCAG |
| | | TTCAACTGGTACGTGGACGGCGTGGAGGTGCACAACGCC |
| | | AAGACCAAGCCCAGAGAGGAGCAGTTCAACAGCACCTA |
| | | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGCCCAGCAGCATCGAGAAGACCATCAGCAAG |
| | | GCCAAGGGCCAGCCCAGAGAGCCCCAGGTGTACACCCT |
| | | GCCCCCCAGCCAGGAGGAGATGACCAAGAACCAGGTGA |
| | | GCCTGACCTGCCTGGTGAAGGGCTTCTACCCCAGCGACA |
| | | TCGCCGTGGAGTGGGAGAGCAACGGCCAGCCCGAGAAC |
| | | AACTACAAGACCACCCCCCCCGTGCTGGACAGCGACGG |
| | | CAGCTTCTTCCTGTACAGCAGACTGACCGTGGACAAGAG |
| | | CAGATGGCAGGAGGGCAACGTGTTCAGCTGCAGCGTGA |
| | | TGCACGAGGCCCTGCACAACCACTACACCCAGAAGAGC |
| | | CTGAGCCTGAGCCTGGGCAAG |
| 76 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG4 HC Constant | CTGCCTGGTGAAGGACTACTTCCCCGAGCCCGTGACCGT |
| | domain (S228P | GAGCTGGAACAGCGGCGCCCTGACCAGCGGCGTGCACA |
| | D265A) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCAAGACCTACACCTGCAACGTGGACCACAAGCCCAG |
| | | CAACACCAAGGTGGACAAGAGAGTGGAGAGCAAGTACG |
| | | GCCCCCCCTGCCCCCCCTGCCCCGCCCCCGAGTTCCTGG |
| | | GCGGCCCCAGCGTGTTCCTGTTCCCCCCCAAGCCCAAGG |
| | | ACACCCTGATGATCAGCAGAACCCCCGAGGTGACCTGCG |
| | | TGGTGGTGGACGTGAGCCAGGAGGACCCCGAGGTGCAG |
| | | TTCAACTGGTACGTGGACGGCGTGGAGGTGCACAACGCC |
| | | AAGACCAAGCCCAGAGAGGAGCAGTTCAACAGCACCTA |
| | | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGGGCAGCAGCATCGAGAAGACCATCAGCAAG |
| | | GCCAAGGGCCAGCCCAGAGAGCCCCAGGTGTACACCCT |
| | | GCCCCCCAGCCAGGAGGAGATGACCAAGAACCAGGTGA |
| | | GCCTGACCTGCCTGGTGAAGGGCTTCTACCCCAGCGACA |
| | | TCGCCGTGGAGTGGGAGAGCAACGGCCAGCCCGAGAAC |
| | | AACTACAAGACCACCCCCCCCGTGCTGGACAGCGACGG |
| | | CAGCTTCTTCCTGTACAGCAGACTGACCGTGGACAAGAG |
| | | CAGATGGCAGGAGGGCAACGTGTTCAGCTGCAGCGTGA |
| | | TGCACGAGGCCCTGCACAACCACTACACCCAGAAGAGC |
| | | GCCAGCACCAAGGGCCCCAGCGTGTTCCCCCTGGCCCCC |
| | | TGCAGCAGAAGCACCAGCGAGAGCACCGCCGCCCTGGG |
| | | CTGCCTGGTGAAGGACTACTTCCCCGAGCCCGTGACCGT |
| | | GAGCTGGAACAGCGGCGCCCTGACCAGCGGCGTGCACA |
| | | CCTTCCCCGCCGTGCTGCAGAGCAGCGGCCTGTACAGCC |
| | | TGAGCAGCGTGGTGACCGTGCCCAGCAGCAGCCTGGGC |
| | | ACCAAGACCTACACCTGCAACGTGGACCACAAGCCCAG |
| | | CAACACCAAGGTGGACAAGAGAGTGGAGAGCAAGTACG |
| | | GCCCCCCCTGCCCCCCCTGCCCCGCCCCCGAGTTCCTGG |
| | | GCGGCCCCAGCGTGTTCCTGTTCCCCCCCAAGCCCAAGG |
| | | ACACCCTGATGATCAGCAGAACCCCCGAGGTGACCTGCG |
| | | TGGTGGTGGCCGTGAGCCAGGAGGACCCCGAGGTGCAG |
| | | TTCAACTGGTACGTGGACGGCGTGGAGGTGCACAACGCC |
| | | AAGACCAAGCCCAGAGAGGAGCAGTTCAACAGCACCTA |
| | | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGCCCAGCAGCATCGAGAAGACCATCAGCAAG |
| | | GCCAAGGGCCAGCCCAGAGAGCCCCAGGTGTACACCCT |
| | | GCCCCCCAGCCAGGAGGAGATGACCAAGAACCAGGTGA |
| | | GCCTGACCTGCCTGGTGAAGGGCTTCTACCCCAGCGACA |
| | | TCGCCGTGGAGTGGGAGAGCAACGGCCAGCCCGAGAAC |
| | | AACTACAAGACCACCCCCCCCGTGCTGGACAGCGACGG |
| | | CAGCTTCTTCCTGTACAGCAGACTGACCGTGGACAAGAG |
| | | CAGATGGCAGGAGGGCAACGTGTTCAGCTGCAGCGTGA |
| | | TGCACGAGGCCCTGCACAACCACTACACCCAGAAGAGC |
| | | CTGAGCCTGAGCCTGGGCAAG |
| 77 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG4 HC Constant | CTGCCTGGTGAAGGACTACTTCCCCGAGCCCGTGACCGT |
| | domain (S228P | GAGCTGGAACAGCGGCGCCCTGACCAGCGGCGTGCACA |
| | D265A N297G) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCAAGACCTACACCTGCAACGTGGACCACAAGCCCAG |
| | | CAACACCAAGGTGGACAAGAGAGTGGAGAGCAAGTACG |
| | | GCCCCCCCTGCCCCCCCTGCCCCGCCCCCGAGTTCCTGG |
| | | GCGGCCCCAGCGTGTTCCTGTTCCCCCCCAAGCCCAAGG |
| | | ACACCCTGATGATCAGCAGAACCCCCGAGGTGACCTGCG |
| | | TGGTGGTGGCCGTGAGCCAGGAGGACCCCGAGGTGCAG |
| | | TTCAACTGGTACGTGGACGGCGTGGAGGTGCACAACGCC |
| | | AAGACCAAGCCCAGAGAGGAGCAGTTCGGCAGCACCTA |
| | | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGCCCAGCAGCATCGAGAAGACCATCAGCAAG |
| | | GCCAAGGGCCAGCCCAGAGAGCCCCAGGTGTACACCCT |
| | | GCCCCCCAGCCAGGAGGAGATGACCAAGAACCAGGTGA |
| | | GCCTGACCTGCCTGGTGAAGGGCTTCTACCCCAGCGACA |
| | | TCGCCGTGGAGTGGGAGAGCAACGGCCAGCCCGAGAAC |
| | | AACTACAAGACCACCCCCCCCGTGCTGGACAGCGACGG |
| | | CAGCTTCTTCCTGTACAGCAGACTGACCGTGGACAAGAG |
| | | CAGATGGCAGGAGGGCAACGTGTTCAGCTGCAGCGTGA |
| | | TGCACGAGGCCCTGCACAACCACTACACCCAGAAGAGC |
| | | CTGAGCCTGAGCCTGGGCAAG |
| 78 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG4 HC Constant | CTGCCTGGTGAAGGACTACTTCCCCGAGCCCGTGACCGT |
| | domain (S228P | GAGCTGGAACAGCGGCGCCCTGACCAGCGGCGTGCACA |
| | N297X, wherein X | CCTTCCCCGCCGTGCTGCAGAGCAGCGGCCTGTACAGCC |
| | is any amino acid | TGAGCAGCGTGGTGACCGTGCCCAGCAGCAGCCTGGGC |
| | other than N) ; | ACCAAGACCTACACCTGCAACGTGGACCACAAGCCCAG |
| | codon 529-531 | CAACACCAAGGTGGACAAGAGAGTGGAGAGCAAGTACG |
| | wherein N is any | GCCCCCCCTGCCCCCCCTGCCCCGCCCCCGAGTTCCTGG |
| | nucleotide with the | GCGGCCCCAGCGTGTTCCTGTTCCCCCCCAAGCCCAAGG |
| | proviso that the | ACACCCTGATGATCAGCAGAACCCCCGAGGTGACCTGCG |
| | codon does not | TGGTGGTGGACGTGAGCCAGGAGGACCCCGAGGTGCAG |
| | encode Asn or form | TTCAACTGGTACGTGGACGGCGTGGAGGTGCACAACGCC |
| | a translation stop | AAGACCAAGCCCAGAGAGGAGCAGTTCNNNAGCACCTA |
| | codon | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGCCCAGCAGCATCGAGAAGACCATCAGCAAG |
| | | GCCAAGGGCCAGCCCAGAGAGCCCCAGGTGTACACCCT |
| | | GCCCCCCAGCCAGGAGGAGATGACCAAGAACCAGGTGA |
| | | GCCTGACCTGCCTGGTGAAGGGCTTCTACCCCAGCGACA |
| | | TCGCCGTGGAGTGGGAGAGCAACGGCCAGCCCGAGAAC |
| | | AACTACAAGACCACCCCCCCCGTGCTGGACAGCGACGG |
| | | CAGCTTCTTCCTGTACAGCAGACTGACCGTGGACAAGAG |
| | | CAGATGGCAGGAGGGCAACGTGTTCAGCTGCAGCGTGA |
| | | TGCACGAGGCCCTGCACAACCACTACACCCAGAAGAGC |
| | | CTGAGCCTGAGCCTGGGCAAG |
| 79 | Nucleotide sequence | AGAACCGTGGCCGCCCCCAGCGTGTTCATCTTCCCCCCC |
| | encoding human LC | AGCGACGAGCAGCTGAAGAGCGGCACCGCCAGCGTGGT |
| | Kappa Constant | GTGCCTGCTGAACAACTTCTACCCCAGAGAGGCCAAGGT |
| | domain | GCAGTGGAAGGTGGACAACGCCCTGCAGAGCGGCAACA |
| | | GCCAGGAGAGCGTGACCGAGCAGGACAGCAAGGACAGC |
| | | ACCTACAGCCTGAGCAGCACCCTGACCCTGAGCAAGGCC |
| | | GACTACGAGAAGCACAAGGTGTACGCCTGCGAGGTGAC |
| | | CCACCAGGGCCTGAGCAGCCCCGTGACCAAGAGCTTCA |
| | | ACAGAGGCGAGTGC |
| 80 | Nucleotide sequence | GCCAGCACCAAGGGCCCCAGCGTGTTCCCCCTGGCCCCC |
| | encoding human | AGCAGCAAGAGCACCAGCGGCGGCACCGCCGCCCTGGG |
| | IgG1 HC Constant | CTGCCTGGTGAAGGACTACTTCCCCGAGCCCGTGACCGT |
| | domain | GAGCTGGAACAGCGGCGCCCTGACCAGCGGCGTGCACA |
| | (N297A/D356E/L358M) | CCTTCCCCGCCGTGCTGCAGAGCAGCGGCCTGTACAGCC |
| | | TGAGCAGCGTGGTGACCGTGCCCAGCAGCAGCCTGGGC |
| | | ACCCAGACCTACATCTGCAACGTGAACCACAAGCCCAGC |
| | | AACACCAAGGTGGACAAGAAGGTGGAGCCCAAGAGCTG |
| | | CGACAAGACCCACACCTGCCCCCCCTGCCCCGCCCCCGA |
| | | GCTGCTGGGCGGCCCCAGCGTGTTCCTGTTCCCCCCCAA |
| | | GCCCAAGGACACCCTGATGATCAGCAGAACCCCCGAGG |
| | | TGACCTGCGTGGTGGTGGACGTGAGCCACGAGGACCCC |
| | | GAGGTGAAGTTCAACTGGTACGTGGACGGCGTGGAGGT |
| | | GCACAACGCCAAGACCAAGCCCAGAGAGGAGCAGTACG |
| | | CCAGCACCTACAGAGTGGTGAGCGTGCTGACCGTGCTGC |
| | | ACCAGGACTGGCTGAACGGCAAGGAGTACAAGTGCAAG |
| | | GTGAGCAACAAGGCCCTGCCCGCCCCCATCGAGAAGAC |
| | | CATCAGCAAGGCCAAGGGCCAGCCCAGAGAGCCCCAGG |
| | | TGTACACCCTGCCCCCCAGCAGAGAGGAGATGACCAAG |
| | | AACCAGGTGAGCCTGACCTGCCTGGTGAAGGGCTTCTAC |
| | | CCCAGCGACATCGCCGTGGAGTGGGAGAGCAACGGCCA |
| | | GCCCGAGAACAACTACAAGACCACCCCCCCCGTGCTGG |
| | | ACAGCGACGGCAGCTTCTTCCTGTACAGCAAGCTGACCG |
| | | TGGACAAGAGCAGATGGCAGCAGGGCAACGTGTTCAGC |
| | | TGCAGCGTGATGCACGAGGCCCTGCACAACCACTACACC |
| | | CAGAAGAGCCTGAGCCTGAGCCCCGGCAAG |
| 81 | Nucleotide sequence | GCCAGCACCAAGGGCCCCAGCGTGTTCCCCCTGGCCCCC |
| | encoding human | TGCAGCAGAAGCACCAGCGAGAGCACCGCCGCCCTGGG |
| | IgG1 HC Constant | CTGCCTGGTGAAGGACTACTTCCCCGAGCCCGTGACCGT |
| | domain | GAGCTGGAACAGCGGCGCCCTGACCAGCGGCGTGCACA |
| | (L234F/L235E/P331 | CCTTCCCCGCCGTGCTGCAGAGCAGCGGCCTGTACAGCC |
| | S/D356E/L358M) | TGAGCAGCGTGGTGACCGTGCCCAGCAGCAGCCTGGGC |
| | | ACCAAGACCTACACCTGCAACGTGGACCACAAGCCCAG |
| | | CAACACCAAGGTGGACAAGAGAGTGGAGAGCAAGTACG |
| | | GCCCCCCCTGCCCCCCCTGCCCCGCCCCCGAGTTCCTGG |
| | | GCGGCCCCAGCGTGTTCCTGTTCCCCCCCAAGCCCAAGG |
| | | ACACCCTGATGATCAGCAGAACCCCCGAGGTGACCTGCG |
| | | TGGTGGTGGACGTGAGCCAGGAGGACCCCGAGGTGCAG |
| | | TTCAACTGGTACGTGGACGGCGTGGAGGTGCACAACGCC |
| | | AAGACCAAGCCCAGAGAGGAGCAGTTCAACAGCACCTA |
| | | CAGAGTGGTGAGCGTGCTGACCGTGCTGCACCAGGACTG |
| | | GCTGAACGGCAAGGAGTACAAGTGCAAGGTGAGCAACA |
| | | AGGGCCTGCCCAGCAGCATCGAGAAGACCATCAGCAAG |
| | | GCCAAGGGCCAGCCCAGAGAGCCCCAGGTGTACACCCT |
| | | GCCCCCCAGCCTGACCTGCCTGGTGAAGGGCTTCTACCC |
| | | CAGCGACATCGCCGTGGAGTGGGAGAGCAACGGCCAGC |
| | | CCGAGAACAACTACAAGACCACCCCCCCCGTGCTGGAC |
| | | AGCGACGGCAGCTTCTTCCTGTACAGCAGACTGACCGTG |
| | | GACAAGAGCAGATGGCAGGAGGGCAACGTGTTCAGCTG |
| | | CAGCGTGATGCACGAGGCCCTGCACAACCACTACACCCA |
| | | GAAGAGCCTGAGCCTGAGCCTGGGCAAG |
| 82 | Nucleotide sequence | GGCCAGCCCAAGGCCAACCCCACCGTGACCCTGTTCCCC |
| | encoding human LC | CCCAGCAGCGAGGAGCTGCAGGCCAACAAGGCCACCCT |
| | lambda Constant | GGTGTGCCTGATCAGCGACTTCTACCCCGGCGCCGTGAC |
| | domain | CGTGGCCTGGAAGGCCGACGGCAGCCCCGTGAAGGCCG |
| | | GCGTGGAGACCACCAAGCCCAGCAAGCAGAGCAACAAC |
| | | AAGTACGCCGCCAGCAGCTACCTGAGCCTGACCCCCGAG |
| | | CAGTGGAAGAGCCACAGAAGCTACAGCTGCCAGGTGAC |
| | | CCACGAGGGCAGCACCGTGGAGAAGACCGTGGCCCCCA |
| | | CCGAGTGCAGC |
| 83 | Leader peptide A | MSVPTQVLGLLLLWLTDARC |
| 84 | Leader peptide B | MEWSWVFLFFLSVTTGVHS |
| 85 | Nucleotide sequence | ATGAGCGTGCCCACCCAGGTGCTGGGCCTGCTGCTGCTG |
| | encoding leader | TGGCTGACCGACGCCAGATGC |
| | peptide A | |
| 86 | Nucleotide sequence | ATGGAGTGGAGCTGGGTGTTCCTGTTCTTCCTGAGCGTG |
| | encoding leader | ACCACCGGCGTGCACAGC |
| | peptide B | |

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

1. An Arginase 1 binder comprising:
(a) three complementarity determining regions (CDRs) of an antibody heavy chain variable domain (VH) comprising the amino acid sequence set forth for VH1 in SEQ ID NO: 2 or VH2 in SEQ ID NO: 3; and
(b) the three CDRs of an antibody light chain variable domain (VL) comprising the amino acid sequence set forth for VL1 in SEQ ID NO: 4 or VL2 in SEQ ID NO: 5.

2. The Arginase 1 binder of claim 1, wherein the Arginase 1 binder specifically binds to two arginase 1 trimers to form a complex comprising three Arginase 1 binders and two arginase trimers, and inhibits arginase 1 activity.

3. The Arginase 1 binder of claim 1, wherein
(a) the VH CDRs comprise a VH1-CDR1 or VH2-CDR1 comprising the amino acid sequence set forth in SEQ ID NO: 6, a VH1-CDR2 or VH2-CDR2 comprising the amino acid sequence set forth in SEQ ID NO: 7, and a VH1-CDR3 or VH2-CDR3 comprising the amino acid sequence set forth in SEQ ID NO: 8, and
(b) the VL CDRs comprise a VL1-CDR1 or VL2-CDR1 comprising the amino acid sequence set forth in SEQ ID NO: 22, a VL1-CDR2 or VL2-CDR2 comprising the amino acid sequence set forth in SEQ ID NO: 23, and a VL1-CDR3 or VL2-CDR3 comprising the amino acid sequence set forth in SEQ ID NO: 24;
wherein the CDR sequences are defined by Kabat.

4. The Arginase 1 binder of claim 1, wherein the Arginase 1 binder comprises an antibody or antigen-binding fragment VH1 comprising the amino acid sequence set forth in SEQ ID NO: 2 and a VL1 comprising the amino acid sequence set forth in SEQ ID NO: 4.

5. The Arginase 1 binder of claim 1, wherein the Arginase 1 binder comprises an antibody or antigen-binding fragment VH2 comprising the amino acid sequence set forth in SEQ ID NO: 3 and a VL2 comprising the amino acid sequence set forth in SEQ ID NO: 5.

6. The Arginase 1 binder of claim 1, wherein the antibody further comprises a heavy chain constant domain of the IgG1, IgG2, IgG3, or IgG4 isotype.

7. The Arginase 1 binder of claim 6, wherein the heavy chain constant domain of the IgG1, IgG2, IgG3, or IgG4 isotype comprises an Fc domain comprising one or more mutations that render the Fc domain effector-silent.

8. An Arginase 1 binder that is an antibody or antigen binding fragment comprising two identical Fabs, a first Fab comprising a first heavy chain variable domain (VH) and a first light chain variable domain (VL) and a second Fab comprising a second VH and a second VL,
wherein the Arginase 1 binder binds to two arginase 1 trimers, a first arginase 1 trimer and a second arginase 1 trimer, each comprising three arginase 1 monomers,
wherein the first Fab binds to an epitope of two adjacent monomers of the first trimer and the second Fab binds to an epitope of two adjacent monomers of the second trimer,
wherein (i) the VH of the first Fab binds to a portion of the epitope located solely on one monomer of two adjacent monomers of the first arginase 1 trimer and the VL of the first Fab binds to a portion of the epitope that spans the two adjacent monomers of the first arginase 1 trimer, and (ii) the VH of the second Fab binds to a portion of the epitope located solely on one monomer of two adjacent monomers of the second arginase 1 trimer and the VL of the second Fab binds to a portion of the epitope that spans the two adjacent monomers of the second arginase 1 trimer and; and
wherein the VH is VH1 or VH2 and the VL is VL1 or VL2 with the proviso that when the VH is VH1 then VL is VL1 and when the VH is VH2 then VL is VL2.

9. The Arginase 1 binder of claim 8, wherein
(a) the VH of the first Fab and the second Fab each binds to amino acids Glu26, Lys33, Pro280, Pro286, Glu26, Ser281, Gly283, Lys284, Thr285, Glu287, Ala34, Thr290, Val293, and Glu38 of one monomer of the two adjacent monomers of the first arginase 1 trimer and second arginase 1 trimer, respectively; and
(b) the VL of the first Fab and the second Fab each binds to
(i) amino acids Glu42, Lys39, Glu38, Lys33, Ala34, Gly35, Lys41, Lys39, Glu287, Thr290, and Asn294 of a monomer (the first monomer) of the two adjacent monomers of the first arginase 1 trimer and second arginase 1 trimer, respectively, and
(ii) amino acids Arg21, Lys68, Gly245, Pro247, Asp181, Val182, Asp183, Pro184, Thr136, and Lys284 of another monomer (the second monomer) of the two adjacent monomers of the first arginase 1 trimer and second arginase 1 trimer, respectively.

10. The Arginase 1 binder of claim 8, wherein the Arginase 1 binder binds two arginase 1 trimers to form a complex comprising a three to two ratio of Arginase 1 binder to arginase 1 trimer.

11. A composition comprising an Arginase 1 binder claims 1 and a pharmaceutically acceptable carrier.

12. A method for treating cancer or proliferative disease in an individual in need thereof comprising:
administering to the individual a therapeutically effective amount an Arginase 1 binder of claims 1 to treat the cancer or a proliferative disease.

13. An arginase 1 binder of claims 1 for treatment of cancer or proliferative disease.

14. (canceled)

15. A combination therapy for treating cancer or proliferative disease comprising an arginase 1 binder of claims 1 and a therapeutic agent.

16. The combination therapy of claim 15, wherein the therapeutic agent is a chemotherapy agent or a therapeutic antibody.

17. The combination therapy of claim 16, wherein the antibody is an anti-PD1 or anti-PD-L1 antibody.

18. A nucleic acid molecule encoding the VH1 or VH2 of the Arginase 1 binder of claims 1 and/or VL1 or VL2 of the Arginase 1 binder of claim 1.

19. An expression vector comprising one or more of the nucleic acid molecules of claim 18.

20. A host cell comprising the expression vector of claim 19.

21. A method for producing an Arginase 1 binder comprising:
(a) providing the host cell of claim 20;
(b) cultivating the host cell in a medium under conditions suitable for expressing the Arginase 1 binder; and
(c) isolating the Arginase 1 binder from the medium.


SEQUENCE LISTING (86 sequences)

SEQ ID NO: 1 - protein, 322 aa, Homo Sapiens
MSAKSRTIGIIGAPFSKGQPRGGVEEGPTVLRKAGLLEKLKEQECDVKDYGDLPFADIPNDSPFQIVKNPRSVGKASEQLAGKVAEVKKNGRISLVLGGDHSLAIGSISGHARVHPDLGVIWVDAHTDINTPLTTTSGNLHGQPVSFLLKELKGKIPDVPGFSWVTPCISAKDIVYIGLRDVDPGEHYILKTLGIKYFSMTEVDRLGIGKVMEETLSYLLGRKKRPIHLSFDVDGLDPSFTPATGTPVVGGLTYREGLYITEEIYKTGLLSGLDIMEVNPSLGKTPEEVTRTVNTAVAITLACFGLAREGNHKPIDYLNPPK

SEQ ID NO: 2 - protein, 116 aa, Artificial Sequence
  VH1 (mouse)
EVQLVQSGAEVKKPGASVKVSCKASGYTFTDYYMNWVRQAPGQGLEWIGVISPYNGGTTYNQKFKGKATLTVDKSTSTAYMELSSLRSEDTAVYYCVYDLYYFDYWGQGTLVTVSS

SEQ ID NO: 3 - protein, 116 aa, Artificial Sequence
  VH2 (humanized VH1)
EVQLQQSGPVLVKPGASVKMSCKASEYTFTDYYMNWVKQSHGRSLEWIGVISPYNGGTTYNQKFKGKATLTVDKSSSTAYMELNSLTSEDSAVYYCVYDLYYFDYWGQGTTLTVSS

SEQ ID NO: 4 - protein, 107 aa, Artificial Sequence
  VL1 (mouse)
EIVLTQSPATLSLSPGERATLSCRASQRISNDLHWYQQKPGQAPRLLIKYASQSISGIPSRFSGSGSGTDFTLTISSLEPEDFAVYYCQQSNSWPRTFGGGTKLEIK

SEQ ID NO: 5 - protein, 107 aa, Artificial Sequence
  VL2 (humanized VL1)
NIVLTQSPATLSVTPGDSVSLSCRASQRISNDLHWYQQKSHESPRLLIKYASQSISGIPSRFSGSGSGTDFTLSINSVETEDFGMYFCQQSNSWPRTFGGGTKLEIK

SEQ ID NO: 6 - protein, 5 aa, Artificial Sequence
  VH1-CDR1 and VH2-CDR1 Kabat (mouse and humanized)
DYYMN

SEQ ID NO: 7 - protein, 17 aa, Artificial Sequence
  VH1-CDR2 and VH2-CDR2 Kabat/Kabat+Chothia (mouse and humanized)
VISPYNGGTTYNQKFKG

SEQ ID NO: 8 - protein, 7 aa, Artificial Sequence
  VH1-CDR3 and VH2-CDR3 Kabat/Chothia/Kabat+Chothia/AbM (mouse and humanized)
DLYYFDY

SEQ ID NO: 9 - protein, 7 aa, Artificial Sequence
  VH1-CDR1 Chothia (mouse)
GYTFTDY

SEQ ID NO: 10 - protein, 7 aa, Artificial Sequence
  VH2-CDR1 Chothia (humanized)
EYTFTDY

SEQ ID NO: 11 - protein, 6 aa, Artificial Sequence
  VH1-CDR2 and VH2-CDR2 Chothia (mouse and humanized)
SPYNGG

SEQ ID NO: 12 - protein, 10 aa, Artificial Sequence
  VH1-CDR1 and VH2-CDR1 Kabat+Chothia/AbM (mouse)
GYTFTDYYMN

SEQ ID NO: 13 - protein, 10 aa, Artificial Sequence
  VH2-CDR1 Kabat+Chothia/AbM (humanized)
EYTFTDYYMN

SEQ ID NO: 14 - protein, 10 aa, Artificial Sequence
  VH1-CDR2 and VH2-CDR2 AbM (mouse and humanized)
VISPYNGGTT

SEQ ID NO: 15 - protein, 8 aa, Artificial Sequence
  VH1-CDR1 IMGT (mouse)
GYTFTDYY

SEQ ID NO: 16 - protein, 8 aa, Artificial Sequence
  VH2-CDR1 IMGT (humanized)
EYTFTDYY

SEQ ID NO: 17 - protein, 7 aa, Artificial Sequence
  VH1-CDR2 and VH2-CDR2 IMGT (mouse and humanized)
ISPYNGG

SEQ ID NO: 18 - protein, 9 aa, Artificial Sequence
  VH1-CDR3 and VH2-CDR3 IMGT (mouse and humanized)
VYDLYYFDY

SEQ ID NO: 19 - protein, 6 aa, Artificial Sequence
  VH1-CDR1 and VH2-CDR1 Contact (mouse and humanized)
TDYYMN

SEQ ID NO: 20 - protein, 13 aa, Artificial Sequence
  VH1-CDR2 and VH2-CDR2 Contact (mouse and humanized)
WIGVISPYNGGTT

SEQ ID NO: 21 - protein, 8 aa, Artificial Sequence
  VH1-CDR3 and VH2-CDR3 Contact (mouse and humanized)
VYDLYYFD

SEQ ID NO: 22 - protein, 11 aa, Artificial Sequence
  VL1-CDR1 and VL2-CDR1 Kabat/Chothia/Kabat+Chothia/AbM (mouse and humanized)
RASQRISNDLH

SEQ ID NO: 23 - protein, 7 aa, Artificial Sequence
  VL1-CDR2 and VL2-CDR2 Kabat/Chothia/Kabat+Chothia/AbM (mouse and humanized)
YASQSIS

SEQ ID NO: 24 - protein, 9 aa, Artificial Sequence
  VL1-CDR3 and VL2-CDR3 Kabat/Chothia/Kabat+Chothia/AbM/IMGT (mouse and humanized)
QQSNSWPRT

SEQ ID NO: 25 - protein, 6 aa, Artificial Sequence
  VL1-CDR1 and VL2-CDR1 IMGT (mouse and humanized)
QRISND

SEQ ID NO: 26 - protein, 7 aa, Artificial Sequence
  VL1-CDR1 and VL2-CDR1 Contact (mouse and humanized)
SNDLHWY

SEQ ID NO: 27 - protein, 10 aa, Artificial Sequence
  VL1-CDR2 and VL2-CDR2 Contact (mouse and humanized)
LLIKYASQSI

SEQ ID NO: 28 - protein, 8 aa, Artificial Sequence
  VL1-CDR3 and VL2-CDR3 Contact (mouse and humanized)
QQSNSWPR

SEQ ID NO: 29 - protein, 330 aa, Artificial Sequence [sequence omitted]

SEQ ID NO: 30 - protein, 330 aa, Artificial Sequence
  Human IgG1 HC Constant domain (L234A L235A D265S)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 330 aa, Artificial Sequence
  Human IgG1 HC Constant domain (L234A L235A P329G)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 330 aa, Artificial Sequence
  Human IgG1 HC Constant domain (L235E)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 330 aa, Artificial Sequence
  Human IgG1 HC Constant domain (D265A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 330 aa, Artificial Sequence
  Human IgG1 HC Constant domain (D265A N297G)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYGSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 330 aa, Artificial Sequence
  Human IgG1 HC Constant domain (E233A/L235A)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPALAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 330 aa, Artificial Sequence
  Human IgG1 HC Constant domain (N297X
  MISC_FEATURE (180)..(180): Xaa is any amino acid other than Asn
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYXSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 326 aa, Artificial Sequence
  Human IgG2 HC Constant domain
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 38 - protein, 326 aa, Artificial Sequence
  Human IgG2 HC Constant domain (D265S)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVSVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 39 - protein, 326 aa, Artificial Sequence
  Human IgG2 HC Constant domain (P329G)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLGAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 40 - protein, 326 aa, Artificial Sequence
  Human IgG2 HC Constant domain (D265A)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVAVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 41 - protein, 326 aa, Artificial Sequence
  Human IgG2 HC Constant domain (D265A N297G)
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVAVSHEDPEVQFNWYVDGVEVHNAKTKPREEQFGSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 42 - protein, 326 aa, Artificial Sequence
  Human IgG2 HC Constant domain (N297X)
  MISC_FEATURE (176)..(176): Xaa is any amino acid other than Asn
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFXSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 43 - protein, 326 aa, Artificial Sequence
  Human IgG2 HC Constant domain (V234A G237A P238S H268A V309L A330S P331S X378S/A)
  misc_feature (257)..(257): Xaa can be any naturally occurring amino acid
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPAAASSVFLFPPKPKDTLMISRTPEVTCVVVDVSAEDPEVQFNWYVDGVEVHNAKTKPREEQFNSTFRVVSVLTVLHQDWLNGKEYKCKVSNKGLPSSIEKTISKTKGQPREPQVYTLPPSREEMTKNQVSLTCLVKGFYPSDIXVEWESNGQPENNYKTTPPMLDSDGSFFLYSKLTVDKSRWQQGNVFSCSVMHEALHNHYTQKSLSLSPGK

SEQ ID NO: 44 - protein, 327 aa, Artificial Sequence
  Human IgG4 HC Constant domain (S228P)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 327 aa, Artificial Sequence
  Human IgG4 HC Constant domain (S228P P329G)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 327 aa, Artificial Sequence
  Human IgG4 HC Constant domain (S228P D265A)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVAVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 327 aa, Artificial Sequence
  Human IgG4 HC Constant domain (S228P D265A N297G)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVAVSQEDPEVQFNWYVDGVEVHNAKTKPREEQFGSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 327 aa, Artificial Sequence
  Human IgG4 HC Constant domain (S228P N297X)
  MISC_FEATURE (177)..(177): Xaa is any amino acid other than Asn
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFXSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 49 - protein, 107 aa, Artificial Sequence [sequence omitted]

SEQ ID NO: 50 - protein, 330 aa, Artificial Sequence
  Human IgG1 HC Constant domain (N297A/D356E/L358M)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 317 aa, Artificial Sequence
  Human IgG1 HC Constant domain (L234F/L235E/P331S/D356E/L358M)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - protein, 106 aa, Artificial Sequence
  Human LC lambda Constant domain
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 53 - protein, 324 aa, Artificial Sequence
  Mouse HC IgG1 (D265A) constant domain
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 54 - protein, 330 aa, Artificial Sequence
  Mouse HC IgG2 constant domain
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 55 - protein, 107 aa, Artificial Sequence
  Mouse LC kappa constant domain
RTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 56 - DNA, 375 bp, Artificial Sequence
  Nucleotide sequence encoding VH1
caggtgcagctggtgcagagcggcgccgaggtgaagaagcccggcgccagcgtgaaggtgagctgcaaggccagcggctacaccttcaccaactacggcatcagctgggtgagacaggcccccggccagggcctggagtggatgggctggatcagcgcctacaacggcaacaccaactacgcccagaagctgcagggcagagtgaccatgaccaccgacaccagcaccagcaccgcctacatggagctgagaagcctgagaagcgacgacaccgccgtgtactactgcgccagagagggcgcctacggctacagaagcccctaccacaactggttcgacccctggggccagggcaccctggtgaccgtgagcagc

SEQ ID NO: 57 - DNA, 375 bp, Artificial Sequence
  Nucleotide sequence encoding VH2
gaggtgcagctggtgcagagcggcgccgaggtgaagaagcccggcgccagcgtgaaggtgagctgcaaggccagcggctacaccttcttcaagtacggcatcagctgggtgagacaggcccccggccagggcctggagtggatgggcagcatcagcccctacaccggcgagacccactacgcccagaagctgcagggcagagtgaccatgaccaccgacaccagcaccagcaccgcctacatggagctgagaagcctgagaagcgacgacaccgccgtgtactactgcgccagagagggcgcctacggctacagaagcccctaccagaactggttcgacccctggggccagggcaccctggtgaccgtgagcagc

SEQ ID NO: 58 - DNA, 321 bp, Artificial Sequence
  Nucleotide sequence encoding VL
gagatcgtgatgacccagagccccgccaccctgagcctgagccccggcgagagagccaccctgagctgcagagccagccagagcgtgagcagctacctggcctggtaccagcagaagcccggccaggcccccagactgctgatctacgacgccagcaacagagccaccggcatccccgccagattcagcggcagcggcagcggcaccgacttcaccctgaccatcagcagcctggagcccgaggacttcgccgtgtactactgccagcagcacagcctgctgcccagaaccttcggcggcggcaccaaggtggagatcaag

SEQ ID NO: 59 - DNA, 990 bp, Artificial Sequence
  Nucleotide sequence encoding human IgG1 HC constant domain
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 60 - DNA, 990 bp, Artificial Sequence
  Nucleotide sequence encoding human IgG1 HC Constant domain (L234A L235A D265S)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgaggccgccggcggccccagcgtgttcctgttcccccccaagcccaaggacaccctgatgatcagcagaacccccgaggtgacctgcgtggtggtgagcgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 61 - DNA, 990 bp, Artificial Sequence
  Nucleotide sequence encoding human IgG1 HC Constant domain (L234A L235A P329G)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgaggccgccggcggccccagcgtgttcctgttcccccccaagcccaaggacaccctgatgatcagcagaacccccgaggtgacctgcgtggtggtgagcgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgggcgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 62 - DNA, 990 bp, Artificial Sequence
  Nucleotide sequence encoding human IgG1 HC Constant domain (L235E)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctggagggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 63 - DNA, 990 bp, Artificial Sequence
  Nucleotide sequence encoding human IgG1 HC Constant domain (D265A)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggccgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 64 - DNA, 990 bp, Artificial Sequence
  Nucleotide sequence encoding human IgG1 HC Constant domain (D265A N297G)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggccgtgagccacgaggaccccgaggtgaagttcaactggtacgtggacggcgtggaggtgcacaacgccaagaccaagcccagagaggagcagtacggcagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 65 - DNA, 990 bp, Artificial Sequence
  Nucleotide sequence encoding human IgG1 HC Constant domain (E233A/L235A)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgccctggccggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 66 - DNA, 990 bp, Artificial Sequence
  Nucleotide sequence encoding human IgG1 HC Constant domain (N297X, wherein X is any amino acid other than N)
  misc_feature (538)..(540): Codon wherein each N is any nucleotide with the proviso that the codon does not encode Asn or form a translation stop codon
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacnnnagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 67 - DNA, 978 bp, Artificial Sequence
  Nucleotide sequence encoding human IgG2 HC Constant domain
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtggacgtgagccacgaggaccccgaggtgcagttcaactggtacgtggacggcgtggaggtgcacaacgccaagaccaagcccagagaggagcagttcaacagcaccttcagagtggtgagcgtgctgaccgtggtgcaccaggactggctgaacggcaaggagtacaagtgcaaggtgagcaacaagggcctgcccgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 68 - DNA, 978 bp, Artificial Sequence
  Nucleotide sequence encoding human IgG2 HC Constant domain (D265S)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtgagcgtgagccacgaggaccccgaggtgcagttcaactggtacgtggacggcgtggaggtgcacaacgccaagaccaagcccagagaggagcagttcaacagcaccttcagagtggtgagcgtgctgaccgtggtgcaccaggactggctgaacggcaaggagtacaagtgcaaggtgagcaacaagggcctgcccgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 69 - DNA, 978 bp, Artificial Sequence
  Nucleotide sequence encoding human IgG2 HC Constant domain (P329G)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtggacgtgagccacgaggaccccgaggtgcagttcaactggtacgtggacggcgtggaggtgcacaacgccaagaccaagcccagagaggagcagttcaacagcaccttcagagtggtgagcgtgctgaccgtggtgcaccaggactggctgaacggcaaggagtacaagtgcaaggtgagcaacaagggcctgggcgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 70 - DNA, 978 bp, Artificial Sequence
  Nucleotide sequence encoding human IgG2 HC Constant domain (D265A)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtggccgtgagccacgaggaccccgaggtgcagttcaactggtacgtggacggcgtggaggtgcacaacgccaagaccaagcccagagaggagcagttcaacagcaccttcagagtggtgagcgtgctgaccgtggtgcaccaggactggctgaacggcaaggagtacaagtgcaaggtgagcaacaagggcctgcccgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 71 - DNA, 978 bp, Artificial Sequence
  Nucleotide sequence encoding human IgG2 HC Constant domain (D265A N297G)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtggccgtgagccacgaggaccccgaggtgcagttcaactggtacgtggacggcgtggaggtgcacaacgccaagaccaagcccagagaggagcagttcggcagcaccttcagagtggtgagcgtgctgaccgtggtgcaccaggactggctgaacggcaaggagtacaagtgcaaggtgagcaacaagggcctgcccgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 72 - DNA, 978 bp, Artificial Sequence
  Nucleotide sequence encoding human IgG2 HC Constant domain (N297X, wherein X is any amino acid other than N)
  misc_feature (526)..(528): Codon wherein each N is any nucleotide with the proviso that the codon does not encode Asn or form a translation stop codon
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgtggccggccccagcgtgttcctgttcccccccaagcccaaggacaccctgatgatcagcagaacccccgaggtgacctgcgtggtggtggacgtgagccacgaggaccccgaggtgcagttcaactggtacgtggacggcgtggaggtgcacaacgccaagaccaagcccagagaggagcagttcnnnagcaccttcagagtggtgagcgtgctgaccgtggtgcaccaggactggctgaacggcaaggagtacaagtgcaaggtgagcaacaagggcctgcccgcccccatcgagaagaccatcagcaagaccaagggccagcccagagagccccaggtgtacaccctgccccccagcagagaggagatgaccaagaaccaggtgagcctgacctgcctggtgaagggcttctaccccagcgacatcagcgtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 73 - DNA, 978 bp, Artificial Sequence
  Nucleotide sequence encoding human IgG2 HC Constant domain (V234A G237A P238S H268A V309L A330S P331S X378S/A)
  misc_feature (769)..(771): n is a, c, g, or t
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagagaaagtgctgcgtggagtgccccccctgccccgccccccccgccgccgccagcagcgtgttcctgttcccccccaagcccaaggacaccctgatgatcagcagaacccccgaggtgacctgcgtggtggtggacgtgagcgccgaggaccccgaggtgcagttcaactggtacgtggacggcgtggaggtgcacaacgccaagaccaagcccagagaggagcagttcaacagcaccttcagagtggtgagcgtgctgaccgtgctgcaccaggactggctgaacggcaaggagtacaagtgcaaggtgagcaacaagggcctgcccagcagcatcgagaagaccatcagcaagaccaagggccagcccagagagccccaggtgtacaccctgccccccagcagagaggagatgaccaagaaccaggtgagcctgacctgcctggtgaagggcttctaccccagcgacatcnnngtggagtgggagagcaacggccagcccgagaacaactacaagaccaccccccccatgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 74 - DNA, 981 bp, Artificial Sequence
  Nucleotide sequence encoding human IgG4 HC Constant domain (S228P)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 75 - DNA, 960 bp, Artificial Sequence
  Nucleotide sequence encoding human IgG4 HC Constant domain (S228P P329G)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgggcagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagc

SEQ ID NO: 76 - DNA, 981 bp, Artificial Sequence
  Nucleotide sequence encoding human IgG4 HC Constant domain (S228P D265A)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggccgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 77 - DNA, 981 bp, Artificial Sequence
  Nucleotide sequence encoding human IgG4 HC Constant domain (S228P D265A N297G)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggccgtgagccaggaggaccccgaggtgcagttcaactggtacgtggacggcgtggaggtgcacaacgccaagaccaagcccagagaggagcagttcggcagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 78 - DNA, 981 bp, Artificial Sequence
  Nucleotide sequence encoding human IgG4 HC Constant domain (S228P N297X, wherein X is any amino acid other than N)
  misc_feature (529)..(531): Codon wherein each N is any nucleotide with the proviso that the codon does not encode Asn or form a transaltion stop codon
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcnnnagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagccaggaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 79 - DNA, 321 bp, Artificial Sequence
  Nucleotide sequence encoding human LC Kappa Constant domain
agaaccgtggccgcccccagcgtgttcatcttcccccccagcgacgagcagctgaagagcggcaccgccagcgtggtgtgcctgctgaacaacttctaccccagagaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacagaggcgagtgc

SEQ ID NO: 80 - DNA, 990 bp, Artificial Sequence
  Nucleotide sequence encoding human IgG1 HC Constant domain (N297A/D356E/L358M)
gccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacgccagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 81 - DNA, 951 bp, Artificial Sequence
  Nucleotide sequence encoding human IgG1 HC Constant domain (L234F/L235E/P331S/D356E/L358M)
gccagcaccaagggccccagcgtgttccccctggccccctgcagcagaagcaccagcgagagcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcaccaagacctacacctgcaacgtggaccacaagcccagcaacaccaaggtggacaagagagtggagagcaagtacggccccccctgccccccctgccccgcccccgagttcctgggcggccccagcgtgttcctgttcccccccaagcccaaggacaccctgatgatcagcagaacccccgaggtgacctgcgtggtggtggacgtgagccaggaggaccccgaggtgcagttcaactggtacgtggacggcgtggaggtgcacaacgccaagaccaagcccagagaggagcagttcaacagcacctacagagtggtgagcgtgctgaccgtgctgcaccaggactggctgaacggcaaggagtacaagtgcaaggtgagcaacaagggcctgcccagcagcatcgagaagaccatcagcaaggccaagggccagcccagagagccccaggtgtacaccctgccccccagcctgacctgcctggtgaagggcttctaccccagcgacatcgccgtggagtgggagagcaacggccagcccgagaacaactacaagaccaccccccccgtgctggacagcgacggcagcttcttcctgtacagcagactgaccgtggacaagagcagatggcaggagggcaacgtgttcagctgcagcgtgatgcacgaggccctgcacaaccactacacccagaagagcctgagcctgagcctgggcaag

SEQ ID NO: 82 - DNA, 318 bp, Artificial Sequence
  Nucleotide sequence encoding human LC lambda Constant domain
ggccagcccaaggccaaccccaccgtgaccctgttcccccccagcagcgaggagctgcaggccaacaaggccaccctggtgtgcctgatcagcgacttctaccccggcgccgtgaccgtggcctggaaggccgacggcagccccgtgaaggccggcgtggagaccaccaagcccagcaagcagagcaacaacaagtacgccgccagcagctacctgagcctgacccccgagcagtggaagagccacagaagctacagctgccaggtgacccacgagggcagcaccgtggagaagaccgtggcccccaccgagtgcagc

SEQ ID NO: 83 - protein, 20 aa, Artificial Sequence
  Leader peptide A
MSVPTQVLGLLLLWLTDARC

SEQ ID NO: 84 - protein, 19 aa, Artificial Sequence
  Leader peptide B
MEWSWVFLFFLSVTTGVHS

SEQ ID NO: 85 - DNA, 60 bp, Artificial Sequence
  Nucleotide sequence encoding leader peptide A
atgagcgtgcccacccaggtgctgggcctgctgctgctgtggctgaccgacgccagatgc

SEQ ID NO: 86 - DNA, 57 bp, Artificial Sequence
  Nucleotide sequence encoding leader peptide B
atggagtggagctgggtgttcctgttcttcctgagcgtgaccaccggcgtgcacagc